(19) United States
(12) Patent Application Publication
(10) Pub. No.: US 2017/0037479 A1
(43) Pub. Date: Feb. 9, 2017

(54) MARKERS FOR OVARIAN CANCER AND THE USES THEREOF

(71) Applicant: Agency for Science, Technology and Research, Singapore (SG)

(72) Inventors: Vladimir Kuznetsov, Singapore (SG); Anna Ivshina, Singapore (SG); Ghim Song Ow, Singapore (SG)

(21) Appl. No.: 15/302,661

(22) PCT Filed: Apr. 8, 2015
(86) PCT No.: PCT/SG2015/050066

§ 371 Date: Oct. 7, 2016

(30) Foreign Application Priority Data
Apr. 8, 2014 (SG) 10201401344W

(51) Int. Cl. C12Q 1/68

(52) U.S. Cl. CPC C12Q 1/6886; C12Q 2600/156; C12Q 2600/118; C12Q 2600/106; C12Q 2600/172; C12Q 2600/158

(57) ABSTRACT

The present invention relates to markers for high-grade serous ovarian cancer (HG-SOC) and methods and uses thereof for diagnosing high-grade serous ovarian cancer (HG-SOC) and/or determining the prognosis of a subject suffering from high-grade serous ovarian cancer (HG-SOC) by determining the presence or absence of a mutation in a CHEK2 marker or mutations of markers from a 21-gene panel comprising ADAMTSL3, ATR, CHEK2, ENAH, ERN2, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC. It also relates to the use of markers CHEK2, RPS6KA2 and MLL4 in predicting the risk of developing high-grade ovarian serous ovarian cancer by determining germline mutations in at least one of these three markers.

### FIELD OF THE INVENTION

The present invention relates to biomarkers for high-grade serous ovarian cancer (HG-SOC) and methods and uses thereof for diagnosing high-grade serous ovarian cancer (HG-SOC) and/or determining the prognosis of a subject suffering from high-grade serous ovarian cancer (HG-SOC).

### BACKGROUND OF THE INVENTION

Ovarian cancer, of which high-grade serous ovarian carcinoma (HG-SOC) is the most prevalent, is one of the most lethal gynecological diseases in the world today. High-grade serous ovarian cancer (HG-SOC), a major histologic type of epithelial ovarian cancer (EOC), is a poorly characterized, heterogeneous and lethal disease where somatic mutations of TP53 are common and inherited loss of-function mutations in BRCA1/2 predispose to cancer in 9.5-13% of EOC patients (Bolton et al JAMA 2012 25; 307 (4): 832-90). However, the overall burden of disease due to either inherited or sporadic mutations is not known. Despite dramatic progress in high-throughput biotechnology and oncogenomic studies, the genetic background of this complex disease is poorly understood and the biomarkers for early detection, differential diagnostics, prognostic and disease prediction have not been implemented in clinical practices. Patients diagnosed with HG-SOC are confronted with a grim statistic that only 30% of them would survive beyond five years after initial diagnosis, even with the standard chemotherapy and radiotherapy. The reasons are likely due to high tumor heterogeneity, unknown tissue source site, asymptomatic tumor growth, late clinical detection and diagnosis, as well as high susceptibility to recurrence after primary chemotherapy.

In fact, the heterogeneity of HG-SOC tumors and the absence of reliable early detection, prognosis and predictive biomarkers, means that clinical status of the patients is varied and the tumors often respond poorly to standard therapy. Therefore, identification of high confidence molecular markers for risk assessment and risk of disease development/recurrence becomes important in various areas ranging from prophylactic to patient clinical management. Therefore, patient stratification based on their survival patterns becomes important in various areas ranging from patient clinical management, to scientific discovery of specific tumor subtypes.

Recent technological advances have facilitated the study of this complex disease, and high-grade serous ovarian carcinoma (HG-SOC) was one of the cancer diseases that have been comprehensively investigated by The Cancer Genome Atlas (TCGA) Research Network. The results of these studies showed that via expression profiling of mRNA data, patients can be classified into four biologically meaningful and distinct tumor/gene subgroups: differentiated, immunoreactive, mesenchymal or proliferative (TCGA research network Nature 2011 Vol 474; 609-15). However, survival analysis did not showed significant differences between these transcriptional sub-types in the TCGA data set. Based on meta-analysis of miRNA and mRNA expression profiles of the TCGA and several other cohorts, HG-SOC patients have been reliably categorized into three prognostic subgroups in which patient's overall survival correlates with specific pathways and treatment outcome (Tang et al. Int J Cancer. 2014; 134 (2): 306-18). Despite the concentrated research effort the information relating to a patent with HG-SOC is no better today than 10 years ago as there is no clinically approved prognostic available.

Recent mutational studies of HG-SOC of the TCGA patient cohort revealed mutated genes such as TP53, NF1, RB1, FAT3, CSMD3, GABRA6, CDK12, BRCA1, BRCA2, SMARCB1, KRAS, NRAS, CREBBP and ERBB2. Other mutations of tumor suppressor genes such as BRIP, CHEK2, MRE11A, MSH6, NBN, PALB2, RAD50 and RAD51C were also identified via massive parallel sequencing in another study. However, these and other mutations have not been systematically studied in context of their ability to provide prognosis of HG-SOC clinical outcome. Studies have showed that in HG-SOC, TP53 somatic mutations were reported in almost all HG-SOC patients and while it would be useful in areas such as early diagnosis or risk prediction of developing the disease, its application in patient survival prediction is restricted. Moreover, conventionally “driver” mutations of BRCA1 or BRCA2 were recently reported to be paradoxically associated with better patient survival relative to the wild-type variant. Typically, studies of mutational data with respect to disease etiology, diagnosis or prognosis may be faced with typical statistical issues due to the lack of appropriate and/or high quality tumor samples. The problem can be further exacerbated when mutations of a particular gene or gene variant are rare.

The effects of CHEK2 mutations in ovarian cancer patient cohorts were previously studied whereby, the missense variant of CHEK2 I157T was significantly associated with ovarian cystadenomas, borderline ovarian cancers and low-grade invasive cancers, but not high-grade ovarian cancer (Szymanska-Pasternak et al. Gynecol Oncol. 2006; 102 (3): 429-31). In another study, Baysal et al. performed single nucleotide polymorphism genotyping by pyrosequencing and identified del1100C and A252G variants of CHEK2 (Baysal et al. Gynecol Oncol. 2004; 95 (1): 62-9). However, as the statistical differences of the variant frequencies were insignificant when compared to controls, it was suggested that variations in CHEK2 are not associated with pathogenesis of ovarian cancer. In Russian ovarian cancer patients, the effects of CHEK2 1100delC on ovarian cancer pathogenesis were studied, but no associations were observed (Krylova et al. Herd Cancer Clin Pract. 2007; 5 (3): 153-56). These studies were mainly focused on screening of some well-reported variants of the CHEK2 gene, e.g. del1100C, A252G and I157T. Moreover, in these previous reports, only the association of specific variants with respect to disease pathogenesis were studied by the authors. However, the prognosis of HG-SOC patients due to the effects of CHEK2 mutations is currently unclear or insignificant.

The interconnectivity and interactions of related genes is a common feature of biological processes in either normal or tumor tissues. The many potential genes involved in the biological process or associated with prognostic significance of HG-SOC particularly those capable of patient stratification make identification of such genes a daunting task. Ovarian cancer is a highly lethal disease that accounts for more deaths than any other cancer of the female reproductive system, and ranks fifth in cancer deaths among women. In this aspect, new methods for prediction and identification of cancer risk assessment, stratification, overall survival prognosis, and therapy response prediction for patients with high-grade serous ovarian carcinoma (HG-SOC) are urgently needed.

### SUMMARY OF THE INVENTION

A first aspect of the invention relates to method for determining the prognosis of a patient afflicted by high-grade serous ovarian cancer (HG-SOC) comprising determining the presence or absence of a mutation in a gene selected from CHEK2, ERN2, ADAMTSL3, ATR, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC in a sample obtained from said patient, wherein the presence of a mutation in the ERN2gene is indicative for a favorable prognosis of the patient and the presence of a mutation in any one of the CHEK2, ADAMTSL3, ATR, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC genes is indicative for a unfavorable prognosis of the patient.

Another aspect of the invention relates to a kit for carrying out the method described herein, the kit comprising at least one nucleic acid probe complementary to mRNA of a mutated gene selected from the group consisting of CHEK2, ERN2, ADAMTSL3, ATR, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC.

Another aspect of the invention relates to a method for predicting the risk of a patient developing high-grade serous ovarian cancer (HG-SOC) comprising determining a germ line mutation in a gene selected from the group consisting of CHEK2, RPS6KA2 and MLL4 in a sample obtained from the patient.

Another aspect of the invention relates to a kit for carrying out the diagnostic method comprising at least one nucleic acid probe complementary to mRNA of a mutated gene selected from the group consisting of CHEK2, RPS6KA2 and MLL4.

Other aspects of the invention will be apparent to a person skilled in the art with reference to the following drawings and description of various non-limiting embodiments.

### BRIEF DESCRIPTION OF THE DRAWINGS

In the following description, various embodiments of the invention are described with reference to the following drawings.

FIG. 1: Mutational data of high-grade serous ovarian carcinoma (HG-SOC) downloaded from TCGA data portal.

FIG. 2: Statistical characteristics of mutations in HG-SOC genes. (A) Frequency distribution of mutations in susceptible driving genes. (B) Number of distinct mutations against number of mutated samples. Scatter plot of genes, where the vertical axis corresponds to the number of mutations across all samples and the horizontal axis corresponds to the number of samples with at least one mutation for a given gene. The diagonal represents the hypothetical scenario where number of mutations per sample for each gene is 1. Both axes are log 10-transformed.

FIG. 3: Kappa correlation of patients with CHEK2 mutations against patients with mutations in BRCA1, BRCA2, RPS6KA2 or MLL4 gene. Values in the contingency table represents the number of unique sample IDs corresponding to the row and column labels. Weighted kappa was calculated as the measure of agreement and the significance was estimated by Mantel-Haenszel (MH) test. The calculations were implemented using StatXact-9 (computed weight: quadratic difference, scores: equally spaced).

FIG. 4: Heatmap of germline, LOH or somatic mutations observed for 455 highly mutated genes (mutated in at least 5 patients) and 334 patients. The intensity of the plot corresponds to the number of mutations (inclusive of silent mutations) observed for that gene and patient.

FIG. 5: (A) Frequency of samples mutated along specific sites of the TP53 gene locus (B) mutations identified at specific sites within the various genes.

FIG. 6: (A) Extracted sub-cluster of mutation matrix belonging to 58 genes and 22 patients, arranged via hierarchical clustering (Kendall-tau distance, complete linkage). The intensity of the plot corresponds to the number of mutations (inclusive of silent mutations) observed for that gene and patient. (B) Direct interaction gene network of a subset of 21 genes identified from the mutation sub-cluster.

FIG. 7: Annotation of 58 gene symbols identified in the mutation subcluster.

FIG. 8: Enrichment analysis of the 58 genes in the mutation subcluster via (A) DAVID Bioinformatics, (B) MetaCore pathway analysis, (C) MetaCore process network analysis and (D) MetaCore disease biomarker Analysis.

FIG. 9: Association of 19 direct interacting genes with DNA-damage signaling, repair, apoptosis, cell proliferation or immune processes

FIG. 10: Consolidated CHEK2 information across clinical, copy number variation, mutation and expression datasets

FIG. 11: Kaplan-Meier survival curves of TCGA HG-SOC patients based on the non-silent mutational 706 status of (A) CHEK2, (B) TP53, (C) BRCA1 and (D) MUC16.

FIG. 12: Kappa correlation of (A) CHEK2 mutations and (B) non-silent CHEK2 mutations with therapy resistance. Values in the contingency table represents the number of unique sample IDs corresponding to the row and column labels.

FIG. 13: (A) Patient stratification of 330 TCGA HG-SOC patients based on CHEK2 copy number. (B) CHEK2 expression for samples with CHEK2 deletion, amplification or insignificant alterations. (C) Expression profiles of CHEK2 mRNA across tumor types for 378 samples. The 378 samples were from 8 fallopian tube samples and 370 HG-SOC samples with tumor grade and stage information. (D) Prognostic stratification of 358 HG-SOC patients based on CHEK2 expression data. 12 HG-SOC patients without survival times and events were excluded from the analysis. High CHEK2 mRNA expression was associated with higher-risk whereas low CHEK2 mRNA expression wad associated with lower-risk.

FIG. 14: Co-localization of TCGA mutation sites with known or predicted CHEK2 regions.

FIG. 15: (A) Genomic locus of CHEK2 from the UCSC Genome Browser. The intron-exon-UTR structure of individual isoforms are shown. (B) RNA-seq expression of the CHEK2 isoforms across 263 high-grade serous ovarian carcinoma patients from the TCGA database.

FIG. 16: (A) Locations of DNA mutations along genomic schema of the CHEK2 locus. The exon blocks are numbered sequentially from 5′ to 3′. Inverted triangles represent the locations of mutation on the exon. The numbers above the inverted triangles indicate the number of patients with the mutation (inclusive of synonymous mutations). (B) Locations of the expected mutations on the amino acid sequence. The alphabet in the inverted triangle indicates the reference amino acid residue, whereas the numbers of patients with non-synonymous mutations are shown above the inverted triangle. The numbers in the rectangular blocks indicate the amino acid residues span. (C) A representative crystal structure of the relaxed state of Chk2 protein after computational modeling and molecular dynamics simulation. All Chk2 mutations are represented by colored spheres which indicate the locations of residues corresponding to the DNA mutations after translation. The CHEK2 isoform 1 (NM_007194/NP_009125/096017) was used as the reference isoform. The forkhead-associated (FHA) domain, kinase domain and nuclear localization signal (NLS) are marked in pink, blue and cyan respectively. The Venn diagram compares the number of patients with the observed mutation at two distinct nucleotide positions. Figures are not drawn to scale.

FIG. 17: Prognostic significance of the 21 survival significant genes based on non-silent mutational status (Logrank statistic p-value ≦0.05, #mutated ≧5 and #non-mutated ≧5).

FIG. 18: (A) Venn diagram of common genes between the identified gene mutation cluster and genes whose mutation status are prognostic significant. (B) Prognostic stratification based on based on mutational status of 21-gene signature. (C) Prognostic stratification based on the mutation of the CHEK2 gene and 20-gene signature.

FIG. 19: Kappa correlation of patients classified by the 21 gene mutational signature with therapy resistance. Values in the contingency table represents the number of unique sample IDs corresponding to the row and column labels.

FIG. 20: Annotation of 21 gene symbols in the prognostic signature

FIG. 21: Enrichment analysis of the 21 survival significant genes via (A) DAVID Bioinformatics, (B) MetaCore pathway analysis, (C) MetaCore process network analysis and (D) MetaCore

FIG. 22: Cluster of non-silent mutations of the 21 prognostic genes and 58 patients in the poor prognosis subgroup for (A) germline, LOH and somatic mutations, (B) germline mutations, (C) LOH, and (D) somatic mutations. Genes and patients were ordered via hierarchical clustering (kendall-tau distance and complete linkage).

FIG. 23: Genetic and clinical characteristics of CHEK2-MLL4-RPS6KA2 determined EOC tumor sub-class (G: Germline, S: Somatic, L: LOH)

FIG. 24: (A) Key genes involved in etiology of various ovarian cancer subtypes. (B) Expression of CHEK2 mRNA across HG-SOC samples of tumor grades and stages (denoted in red boxplots). Differential expression between the normal and tumor samples were calculated via Mann-Whitney test.

### DETAILED DESCRIPTION

Integrative bioinformatics and statistical analysis of genome-wide mutational and clinical datasets of HG-SOC patients from TCGA allowed identification of prognostic genes (biomarkers) whose mutation status could stratify patients into distinct survival subgroups. Gene signatures related to poor prognosis of patients, where distinct tumor subgroups are characterized and potentially driven by germline or somatic mutations of these signature genes was also identified.

The identification of novel molecular markers for risk assessment and diseased patient stratification based on their survival patterns becomes important in various areas ranging from discovery of specific tumor classes and subtypes to improved prophylactics, early diagnostics, and clinical management.

A first aspect of the invention relates to method for determining the prognosis of a patient afflicted by high-grade serous ovarian cancer (HG-SOC) comprising determining the presence or absence of a mutation in a gene selected from CHEK2, ERN2, ADAMTSL3, ATR, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC in a sample obtained from said patient, wherein the presence of a mutation in the ERN2gene is indicative for a favorable prognosis of the patient and the presence of a mutation in any one of the CHEK2, ADAMTSL3, ATR, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC genes is indicative for a unfavorable prognosis of the patient.

These mutated genes or marker genes can be detected in tissue and/or body fluid samples, e.g., in a blood sample, and thus provide for a novel method for the prognosis of a patient afflicted by HG-SOC. As such a method does not require expensive equipment, the new method can be carried out by any physician. Preferably, the gene or marker gene is detected directly, i.e. on the DNA level, or by means of a gene product, including mRNA or a protein. Preferably, the mutations are detected via sequencing methods such as sequencing via Illumina or ABI SOLID sequencing platforms. Any suitable method such as PCT melt techniques may also be suitable for determining mutations or any other methods known in the art for determining sequence variations.

For the detection of the gene markers of the present invention specific binding partners may be employed. In some embodiments, the specific binding partners are useful to detect the presence of a marker in a sample, wherein the marker is a protein or RNA. The marker and its binding partner represent a binding pair of molecules, which interact with each other through any of a variety of molecular forces including, for example, ionic, covalent, hydrophobic, van der Waals, and hydrogen bonding. Preferably, this binding is specific. “Specific binding” means that the members of a binding pair bind preferentially to each other, i.e. usually with a significant higher affinity than to non-specific binding partners. The binding affinity for specific binding partners is thus usually at least 10-fold, preferably at least 100-fold higher than that for non-specific binding partners. The binding partners may also be specific in that they bind the mutated form of the gene product, i.e. the RNA or protein, with higher affinity than the non-mutated form, with the difference preferably being an at least 10-fold increase in affinity.

Determining the prognosis includes risk stratification and prediction of the likelihood of an adverse outcome. This can be made in relation to a certain time period. In various embodiments, the time period is 5 years. An unfavorable or adverse outcome in the sense of the present invention includes deterioration of a patient's condition, for example due to metastasis or death within the 5 years after diagnosis or determination of the prognosis, as described herein. A favorable or positive outcome includes maintenance or improvement of a patient's condition, for example due to responding positively to chemotherapy, such as cisplatin therapy, or survival for 5 years or more.

Overall, the technology can improve patient risk assessment, management and counselling, as well as provide a solution for the optimization of personalized medicine strategy of treating human ovarian cancers in a clinical setting.

In various embodiments, a mutation in the CHEK2 gene is detected. Checkpoint kinase 2 (CHEK2) encodes a nuclear serine/threonine protein kinase involved in cell cycle checkpoint control, DNA damage response signalling and apoptosis regulation. CHEK2. In the presence of DNA damage, CHEK2 phosphorylates downstream cell cycle regulators such as p53, Cdc25 and BRCA1 to activate checkpoint repair or recovery responses, as well as concurrently delay entry into mitosis. Deviation from its normal physiological function may contribute to disease pathogenesis.

In various embodiments the CHEK2 marker gene comprises a sequence as set forth in any one of SEQ ID NO. 1 (NM_001005735), or SEQ ID NO. 67 (NM_001257387), or SEQ ID NO. 68 (NM_007194), or SEQ ID NO. 69 (NM_145862). These sequences are the most common sequence known for CHEK2 and mutations or variations from these standard sequences have been demonstrated here to correlate to the poor survival of a patient afflicted by high-grade serous ovarian cancer. Aberrations of CHEK2 gene were not previously associated with prognosis of overall survival time or therapy response in HG-SOC. The relatively large and well-designed cohort of 334 HG-SOC patients allowed identification of a previously unidentified subclass of patients, with potentially very poor therapy response and overall survival (5 years overall survival rate of 0%). Where a mutation is detected in the CHEK2 marker gene, patients may be counselled on palliative care. This will save the patient the unnecessary expense and pain involved with aggressive treatment with chemotherapy.

In various embodiments, the ADAMTSL3 marker gene comprises a sequence as set forth in any one of SEQ ID NO 5 (NM_001301110) and SEQ ID NO. 71 (NM_207517).

In various embodiments, the ATR marker gene comprises a sequence as set forth in SEQ ID NO. 6 (NM_001184).

In various embodiments, the ENAH marker gene comprises a sequence as set forth in any one of SEQ ID NO. 7 (NM_001008493) and SEQ ID NO. 72 (NM_018212).

In various embodiments, the GLI2 marker gene comprises a sequence as set forth in SEQ ID NO. 8 (NM_005270).

In various embodiments, the GYPB marker gene comprises a sequence as set forth in any one of SEQ ID NO. 9 (NM_001304382) and SEQ ID NO. 73 (NM_002100).

In various embodiments, the KIAA1324L marker gene comprises a sequence as set forth in any one of SEQ ID NO. 10 (NM_001142749), SEQ ID NO. 74 (NM_001291990), SEQ ID NO. 75 (NM_001291991), and SEQ ID NO. 76 (NM_152748).

In various embodiments, the LRRN2 marker gene comprises a sequence as set forth in any one of SEQ ID NO. 11 (NM_006338) and SEQ ID NO. 77 (NM_201630).

In various embodiments, the MAP3K6 marker gene comprises a sequence as set forth in any one of SEQ ID NO. 12 (NM_001297609) and SEQ ID NO. 78 (NM_004672).

In various embodiments, the MAPK15 marker gene comprises a sequence as set forth in SEQ ID NO. 13 (NM_139021).

In various embodiments, the MET marker gene comprises a sequence as set forth in any one of SEQ ID NO. 14 (NM_000245) and SEQ ID NO. 79 (NM_001127500)

In various embodiments, the MLL4 marker gene comprises a sequence as set forth in SEQ ID NO. 4 (NM_014727). The marker may also be called KMT2B.

In various embodiments, the NIPBL marker gene comprises a sequence as set forth in any one one of SEQ ID NO. 15 (NM_015384) and SEQ ID NO. 80 (NM_133433).

In various embodiments, the PCDH15 marker gene comprises a sequence as set forth in any one of SEQ ID NO. 16 (NM_001142763), SEQ ID NO. 81 (NM_001142764), SEQ ID NO. 82 (NM_001142765), SEQ ID NO. 83 (NM_001142766), SEQ ID NO. 84 (NM_001142767), SEQ ID NO. 85 (NM_001142768), SEQ ID NO. 86 (NM_001142769), SEQ ID NO. 87 (NM_001142770), SEQ ID NO. 88 (NM_001142771), SEQ ID NO. 89 (NM_001142772), SEQ ID NO. 90 (NM_001142773), and SEQ ID NO. 91 (NM_033056).

In various embodiments, the PPP1CC marker gene comprises a sequence as set forth in any one of SEQ ID NO. 17 (NM_001244974) and SEQ ID NO. 92 (NM_002710).

In various embodiments, the PTCH1 marker gene comprises a sequence as set forth in any one of SEQ ID NO. 18 (NM_000264), SEQ ID NO. 93 (NM_001083602), SEQ ID NO. 94 (NM_001083603), SEQ ID NO. 95 (NM_001083604), SEQ ID NO. 96 (NM_001083605), SEQ ID NO. 97 (NM_001083606), and SEQ ID NO. 98 (NM_001083607).

In various embodiments, the PTK2B marker gene comprises a sequence as set forth in any one of SEQ ID NO. 19 (NM_004103), SEQ ID NO. 99 (NM_173174), SEQ ID NO. 100 (NM_173175), and SEQ ID NO. 101 (NM_173176).

In various embodiments, the RPS6KA2 marker gene comprises a sequence as set forth in any one of SEQ ID NO. 3 (NM_001006932), and SEQ ID NO. 70 (NM_021135).

In various embodiments, the RSU1 marker gene comprises a sequence as set forth in SEQ ID NO. 20 (NM_012425), and SEQ ID NO. 102 (NM_152724).

In various embodiments, the TNC marker gene comprises a sequence as set forth in SEQ ID NO. 21 (NM_002160).

All the afore-mentioned sequences are the respective wildtype sequences that can be used as a reference to detect mutations in these genes. The codes in brackets refer to their respective databank entry numbers.

In various embodiments, the mutation in the ERN2 marker gene is indicative of a favorable therapeutic outcome of the patient. In various embodiments the ERN2 marker comprises the nucleic acid sequence set forth in SEQ ID NO. 2 (NM_033266). This sequence is the most common sequence known for ERN2 and mutations or variations from this standard wildtype sequence have been demonstrated here to correlate to the better survival of a patient afflicted by high-grade serous ovarian cancer, with the overall 5 year survival rate being 37%. As ERN2 mutations correlate with better overall survival of patients, HG-SOC patients identified to have mutations in the ERN2 marker can be treated with chemotherapy and other treatments such as radiation therapy and resection.

In various embodiments, the method further comprises the step of confirming the prognosis by microscopic analysis of an ovarian tissue biopsy or by ultrasound or any other means known in the art of confirming the prognosis of ovarian cancer, particularly HG-SOC, in a patient. The ultrasound may be done externally or preferably intra-vaginally to better determine the size of any tumor growth. The method of confirming the prognosis may also include detection of mutations in known markers of ovarian cancer, preferably HG-SOC, such as mutations in TP53, BRCA1 or BRCA2. The results indicate that CHEK2 and BRCA1 are mutually exclusive mutations which may be able to stratify patients who will or will not respond well to chemotherapy. Patients with a mutation of a CHEK2 marker do typically not respond well to chemotherapy.

In various embodiments, the mutation in CHEK2 marker is located in exon 10, 11 or 15 of the CHEK2 marker. In various embodiments the terminal exon 15 of the CHEK2 gene expresses a nuclear localization sequence. CHEK2 mutations in HG-SOC patients are strong adverse indicator of patient survival prognosis and associated with therapy resistance. It is hypothesized without being limited to any theories that it could be due to mutations of the nuclear localization site which prevents the nuclear import of the protein and subsequently leads to haplo-insufficiency. In various embodiments the mutation is located at a sequence position corresponding to a codon that encodes amino acids R346, T383, R406, R519, P522, R535 and/or P536. These amino acids are present in a nuclear localization site of CHEK2.

In various embodiments, the methods of the invention can further comprise determining the presence of a mutation in one or more additional markers of those listed above. If one or more additional mutated marker genes are detected, this may increase the accuracy of the method. In certain embodiments of the methods of the invention, mutations in at least 2, 3, 4, 5, 6, 7, 8, 9, 10, 20, 30, 40, 45, 50, or 58 or more additional markers are determined.

In various embodiments, where a mutation in the CHEK2 marker is determined, the method further comprises determining a mutation in any one of the genes selected from the group consisting of ABCA3, ADAM15, ADAMTSL3, ALK, ANKHD1-EIF4EBP3, ANKMY2, ANXA7, ASPM, CDC27, CHD6, CHL1, DPYSL4, ENAH, EP400, ERBB2IP, FN1, FOXO3, GCLC, GLI2, GLI3, GYPB, GZMB, HLA-G, HNF1A, INPP5D, INSR, ITGB2, KIF3B, KIF4B, KTN1, LRRN2, MAD1L1, MAP3K6, MAPK15, MET, MKL1, MLL4, MYO5C, NUMA1, PDGFRA, PHLPP, PIK3C2B, PKP4, PLAGL2, PPARA, PRKCI, PTK2B, RAB3D, ROR2, RPS6KA2, RSU1, SPTB, TBK1, TNK2, TP53, VAV1 and ZC3H11A.

In various embodiments, the ABCA3 marker gene comprises a sequence as set forth in SEQ ID NO. 22 (NM_001089).

In various embodiments, the ADAM15 marker gene comprises a sequence as set forth in SEQ ID NO. 23 (NM_001261464), SEQ ID NO 103 (NM_001261465), SEQ ID NO. 104 (NM_001261466), SEQ ID NO. 105 (NM_003815), SEQ ID NO. 106 (NM_207191), SEQ ID NO. 107 (NM_207194), SEQ ID NO. 108 (NM_207195), SEQ ID NO. 109 (NM_207196), SEQ ID NO. 110 (NM_207197), SEQ ID NO. 111 (NR_048577), SEQ ID NO. 112 (NR_048578), and SEQ ID NO. 113 (NR_048579)

In various embodiments, the ADAMTSL3 marker gene comprises a sequence as set forth in any one of SEQ ID NO 5 (NM_001301110) and SEQ ID NO. 71 (NM_207517).

In various embodiments, the ALK marker gene comprises a sequence as set forth in SEQ ID NO. 24 (NM_004304).

In various embodiments, the ANKHD1-EIF4EBP3 marker gene comprises a sequence as set forth in SEQ ID NO. 25 (NM_020690).

In various embodiments, the ANKMY2 marker gene comprises a sequence as set forth in SEQ ID NO. 26 (NM_020319).

In various embodiments, the ANXA7 marker gene comprises a sequence as set forth in SEQ ID NO. 27 (NM_001156), and SEQ ID NO. 114 (NM_004034).

In various embodiments, the ASPM marker gene comprises a sequence as set forth in SEQ ID NO. 28 (NM_001206846), and SEQ ID NO. 115 (NM_018136).

In various embodiments, the CDC27 marker gene comprises a sequence as set forth in SEQ ID NO. 29 (NM_001114091), SEQ ID NO. 116 (NM_001256), SEQ ID NO. 117 (NM_001293089), and SEQ ID NO. 118 (NM_001293091).

In various embodiments, the CHD6 marker gene comprises a sequence as set forth in SEQ ID NO. 30 (NM_032221).

In various embodiments, the CHL1 marker gene comprises a sequence as set forth in SEQ ID NO. 31 (NM_001253387), SEQ ID NO. 119 (NM_001253388), SEQ ID NO. 120 (NM_006614), and SEQ ID NO. 121 (NR_045572).

In various embodiments, the DPYSL4 marker gene comprises a sequence as set forth in SEQ ID NO. 32 (NM_006426).

In various embodiments, the ENAH marker gene comprises a sequence as set forth in any one of SEQ ID NO. 7 (NM_001008493) and SEQ ID NO. 72 (NM_018212).

In various embodiments, the GLI2 marker gene comprises a sequence as set forth in SEQ ID NO. 8 (NM_005270).

In various embodiments, the EP400 marker gene comprises a sequence as set forth in SEQ ID NO. 33 (NM_015409).

In various embodiments, the ERBB2IP marker gene comprises a sequence as set forth in SEQ ID NO. 34 (NM_001006600), SEQ ID NO. 122 (NM_001253697), SEQ ID NO. 123 (NM_001253698), SEQ ID NO. 124 (NM_001253699), SEQ ID NO. 125 (NM_001253701), and SEQ ID NO. 126 (NM_018695).

In various embodiments, the FN1 marker gene comprises a sequence as set forth in SEQ ID NO. 35 (NM_002026), SEQ ID NO. 127 (NM_054034), SEQ ID NO. 128 (NM_212474), SEQ ID NO. 129 (NM_212476), SEQ ID NO. 130 (NM_212478), and SEQ ID NO. 131 (NM_212482)

In various embodiments, the FOXO3 marker gene comprises a sequence as set forth in SEQ ID NO. 36 (NM_001455), and SEQ ID NO. 132 (NM_201559).

In various embodiments, the GCLC marker gene comprises a sequence as set forth in SEQ ID NO. 37 (NM_001197115), and SEQ ID NO. 133 (NM_001498).

In various embodiments, the GLI3 marker gene comprises a sequence as set forth in SEQ ID NO. 38 (NM_000168).

In various embodiments, the GYPB marker gene comprises a sequence as set forth in any one of SEQ ID NO. 9 (NM_001304382) and SEQ ID NO. 73 (NM_002100).

In various embodiments, the GZMB marker gene comprises a sequence as set forth in SEQ ID NO. 39 (NM_004131).

In various embodiments, the HLA-G marker gene comprises a sequence as set forth in SEQ ID NO. 40 (NM_002127).

In various embodiments, the HNF1A marker gene comprises a sequence as set forth in SEQ ID NO. 41 (NM_000545).

In various embodiments, the INPP5D marker gene comprises a sequence as set forth in SEQ ID NO. 42 (NM_001017915), and SEQ ID NO. 134 (NM_005541).

In various embodiments, the INSR marker gene comprises a sequence as set forth in SEQ ID NO. 43 (NM_000208), and SEQ ID NO. 135 (NM_001079817).

In various embodiments, the ITGB2 marker gene comprises a sequence as set forth in SEQ ID NO. 44 (NM_000211), SEQ ID NO. 136 (NM_001127491), and SEQ ID NO. 137 (NM_001303238).

In various embodiments, the KIF3B marker gene comprises a sequence as set forth in SEQ ID NO. 45 (NM_004798).

In various embodiments, the KIF4B marker gene comprises a sequence as set forth in SEQ ID NO. 46 (NM_001099293).

In various embodiments, the KTN1 marker gene comprises a sequence as set forth in SEQ ID NO. 47 (NM_001079521), SEQ ID NO. 138 (NM_001079522), SEQ ID NO. 139 (NM_001271014), SEQ ID NO. 140 (NM_004986), SEQ ID NO. 141 (NR_073128), and SEQ ID NO. 142 (NR_073129).

In various embodiments, the LRRN2 marker gene comprises a sequence as set forth in any one of SEQ ID NO. 11 (NM_006338) and SEQ ID NO. 77 (NM_201630).

In various embodiments, the MAD1L1 marker gene comprises a sequence as set forth in SEQ ID NO. 48 (NM_001013836), SEQ ID NO. 143 (NM_001013837), SEQ ID NO. 144 (NM_001304523), SEQ ID NO. 145 (NM_001304524), SEQ ID NO. 146 (NM_001304525), and SEQ ID NO. 147 (NM_003550).

In various embodiments, the MAP3K6 marker gene comprises a sequence as set forth in any one of SEQ ID NO. 12 (NM_001297609) and SEQ ID NO. 78 (NM_004672).

In various embodiments, the MAPK15 marker gene comprises a sequence as set forth in SEQ ID NO. 13 (NM_139021).

In various embodiments, the MET marker gene comprises a sequence as set forth in any one of SEQ ID NO. 14 (NM_000245) and SEQ ID NO. 79 (NM_001127500)

In various embodiments, the MKL1 marker gene comprises a sequence as set forth in SEQ ID NO. 49 (NM_001282660), SEQ ID NO. 148 (NM_001282661), SEQ

ID NO. 149 (NM_001282662), and SEQ ID NO. 150 (NM_020831)

In various embodiments, the MLL4 marker gene comprises a sequence as set forth in SEQ ID NO. 4 (NM_014727). The gene may also be called KMT2B.

In various embodiments, the MYO5C marker gene comprises a sequence as set forth in SEQ ID NO. 50 (NM_018728).

In various embodiments, the NUMA1 marker gene comprises a sequence as set forth in SEQ ID NO. 51 (NM_001286561), SEQ ID NO. 151 (NM_006185), and SEQ ID NO. 152 (NR_104476).

In various embodiments, the PDGFRA marker gene comprises a sequence as set forth in SEQ ID NO. 52 (NM_006206).

In various embodiments, the PHLPP marker gene comprises a sequence as set forth in SEQ ID NO. 53 (NM_194449). The gene may also be called PHLPP1.

In various embodiments, the PIK3C2B marker gene comprises a sequence as set forth in SEQ ID NO. 54 (NM_002646).

In various embodiments, the PKP4 marker gene comprises a sequence as set forth in SEQ ID NO. 55 (NM_001005476), SEQ ID NO. 153 (NM_001304969), SEQ ID NO. 154 (NM_001304970), SEQ ID NO. 155 (NM_001304971), and SEQ ID NO. 156 (NM_003628).

In various embodiments, the PLAGL2 marker gene comprises a sequence as set forth in SEQ ID NO. 56 (NM_002657).

In various embodiments, the PPARA marker gene comprises a sequence as set forth in SEQ ID NO. 57 (NM_001001928), SEQ ID NO. 157 (NM_005036)

In various embodiments, the PRKCI marker gene comprises a sequence as set forth in SEQ ID NO. 58 (NM_002740).

In various embodiments, the PTK2B marker gene comprises a sequence as set forth in any one of SEQ ID NO. 19 (NM_004103), SEQ ID NO. 99 (NM_173174), SEQ ID NO. 100 (NM_173175), and SEQ ID NO. 101 (NM_173176).

In various embodiments, the RAB3D marker gene comprises a sequence as set forth in SEQ ID NO. 59 (NM_004283).

In various embodiments, the ROR2 marker gene comprises a sequence as set forth in SEQ ID NO. 60 (NM_004560).

In various embodiments, the RPS6KA2 marker gene comprises a sequence as set forth in any one of SEQ ID NO. 3 (NM_001006932), and SEQ ID NO. 70 (NM_021135).

In various embodiments, the RSU1 marker gene comprises a sequence as set forth in SEQ ID NO. 20 (NM_012425), and SEQ ID NO. 102 (NM_152724).

In various embodiments, the SPTB marker gene comprises a sequence as set forth in SEQ ID NO. 61 (NM_000347), and SEQ ID NO. 158 (NM_001024858).

In various embodiments, the TBK1 marker gene comprises a sequence as set forth in SEQ ID NO. 62 (NM_013254).

In various embodiments, the TNK2 marker gene comprises a sequence as set forth in SEQ ID NO. 63 (NM_001010938), and SEQ ID NO. 159 (NM_005781).

In various embodiments, the TP53 marker gene comprises a sequence as set forth in SEQ ID NO. 64 (NM_000546), SEQ ID NO. 160 (NM_001126112), SEQ ID NO. 161 (NM_001126113), SEQ ID NO. 162 (NM_001126114), SEQ ID NO. 163 (NM_001126115), SEQ ID NO. 164 (NM_001126116), SEQ ID NO. 165 (NM_001126117), SEQ ID NO. 166 (NM_001126118), SEQ ID NO. 167 (NM_001276695), SEQ ID NO. 168 (NM_001276696), SEQ ID NO. 169 (NM_001276697), SEQ ID NO. 170 (NM_001276698), SEQ ID NO. 171 (NM_001276699), SEQ ID NO. 172 (NM_001276760), and SEQ ID NO. 173 (NM_001276761).

In various embodiments, the VAV1 marker gene comprises a sequence as set forth in SEQ ID NO. 65 (NM_001258206), SEQ ID NO. 174 (NM_001258207), and SEQ ID NO175 (NM_005428).

In various embodiments, the ZC3H11A marker gene comprises a sequence as set forth in SEQ ID NO. 66 (NM_014827).

All the afore-mentioned sequences are the respective wildtype sequences that can be used as a reference to detect mutations in these genes. The codes in brackets refer to their respective databank entry numbers.

In various embodiments, determining the presence or absence of a mutation in any one of a panel of marker genes comprises detecting the presence or absence of a mutation of a nucleotide sequence selected from the group consisting of the nucleic acid sequences set forth in SEQ ID NOS 3-5, 7-9, 11-14, 19, 20, 22-73, 77-79 99-175.

In various embodiments, where a mutation in the CHEK2 marker is determined, the method further comprises determining a mutation in any one of the genes selected from the group consisting of ADAMTSL3, ATR, ENAH, ERN2, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC. In some embodiments, mutations in at least 2, 3, 4, 5, 6, 7, 8, 9, 10, 11, 12, 13, 14, 15, 16, 17, 18, 19 or all 20 genes are additionally determined.

In various embodiments, the method further comprises determining the presence of a mutations in each of the following markers ADAMTSL3, ATR, CHEK2, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC. The detection of any mutations in any of the afore-mentioned genes is indicative of a unfavorable therapeutic outcome of the patient. In various embodiments the markers comprise nucleic acid sequences set forth in SEQ ID NOS. 3-21, or 70-102. Mutations in any one of the above-mentioned markers correlate with poorer overall survival of patients.

In various embodiments, the method further comprises determining a mutation in any one of a marker sequence selected from the group consisting of nucleic acid sequences set forth in SEQ ID NOS. 1, 3-21 and 67-102.

In various embodiments, the method comprises determining the presence or absence of a mutation in a panel of gene markers comprising any two or more, three or more, four or more, five or more, six or more, seven or more, eight, or more, nine or more, ten or more, eleven or more, twelve or more, thirteen or more, fourteen or more, fifteen or more, sixteen or more, seventeen or more, eighteen or more, nineteen or more, twenty or more or all 21 of CHEK2, ERN2, ADAMTSL3, ATR, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC. In this embodiment the combined mutational panel or signature comprising 21 genes (DNA and/or mRNA and/or protein) may be used to stratify a cohort of patients into low and high-risk subgroups. HG-SOC patients are classified as low-risk if mutations are observed in ERN2 or no mutations are observed in ADAMTSL3, ATR, CHEK2, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC. HG-SOC patients are classified as high-risk if mutations are not observed in ERN2 and mutations are observed in ADAMTSL3, ATR, CHEK2, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC. In various embodiments, the method may comprises determining the presence of a mutation in a panel of markers comprising the non-mutated sequences as set forth in SEQ ID NOS 1-21.

In various embodiments, the method comprises determining a mutation in a panel of markers comprising ADAMTSL3, ATR, CHEK2, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC wherein a mutation in any one of the panel of gene markers is indicative of an unfavorable therapeutic outcome of the patient. In various embodiments, the method comprises determining the presence or absence of a mutation in a panel of marker having the wildtype sequences set forth in SEQ ID NOS. 1, and 3-21, with the presence of a mutation being indicative for said the subject having an unfavorable prognosis. HG-SOC patients are classified as high-risk if mutations are not observed in ERN2 and mutations are observed in ADAMTSL3, ATR, CHEK2, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC.

In various embodiments a first tumor subtype is determined by a germ line mutation in CHEK2, RPS6KA2 and/or MLL4 marker. In various embodiments a first tumor subtype is determined by detection of the presence of a germ line mutation in a nucleic acid sequence as set forth in any one of SEQ ID NOS 1, 3 and/or 4, with said sequences corresponding to the respective wildtype sequences.

In various embodiments the wildtype sequences for CHEK2 marker gene comprises a sequence as set forth in any one of SEQ ID NO. 1 (NM_001005735), or SEQ ID NO. 67 (NM_001257387), or SEQ ID NO. 68 (NM_007194), or SEQ ID NO. 69 (NM_145862). The wildtype sequences for RPS6KA2 marker gene comprises a sequence as set forth in any one of SEQ ID NO. 3 (NM_001006932), and SEQ ID NO. 70 (NM_021135). The wildtype sequences for MLL4 marker gene comprises a sequence as set forth in SEQ ID NO. 4 (NM_014727). The gene may also be called KMT2B.

In various embodiments, the method comprises determining the presence or absence of a mutation in a panel of gene markers comprising CHEK2, ABCA3, ADAM15, ADAMTSL3, ALK, ANKHD1-EIF4EBP3, ANKMY2, ANXA7, ASPM, CDC27, CHD6, CHEK2, CHL1, DPYSL4, ENAH, EP400, ERBB2IP, FN1, FOXO3, GCLC, GLI2, GLI3, GYPB, GZMB, HLA-G, HNF1A, INPP5D, INSR, ITGB2, KIF3B, KIF4B, KTN1, LRRN2, MAD1L1, MAP3K6, MAPK15, MET, MKL1, MLL4, MYO5C, NUMA1, PDGFRA, PHLPP, PIK3C2B, PKP4, PLAGL2, PPARA, PRKCI, PTK2B, RAB3D, ROR2, RPS6KA2, RSU1, SPTB, TBK1, TNK2, TP53, VAV1 and ZC3H11A. In this embodiment, the combined mutational panel or signature comprises 58 genes which are relatively often mutated in 7% of HG-SOC patients. This panel may be used to identify HG-SOC patients having a poor prognosis. In various embodiments, the method comprises determining the presence or absence of a mutation in a panel of gene markers comprises detecting the presence or absence of a mutation in any one or more of the nucleotide sequences set forth in SEQ ID NOS 3-5, 7-9, 11-14, 19, 20, 22-73, 77-79 99-175.

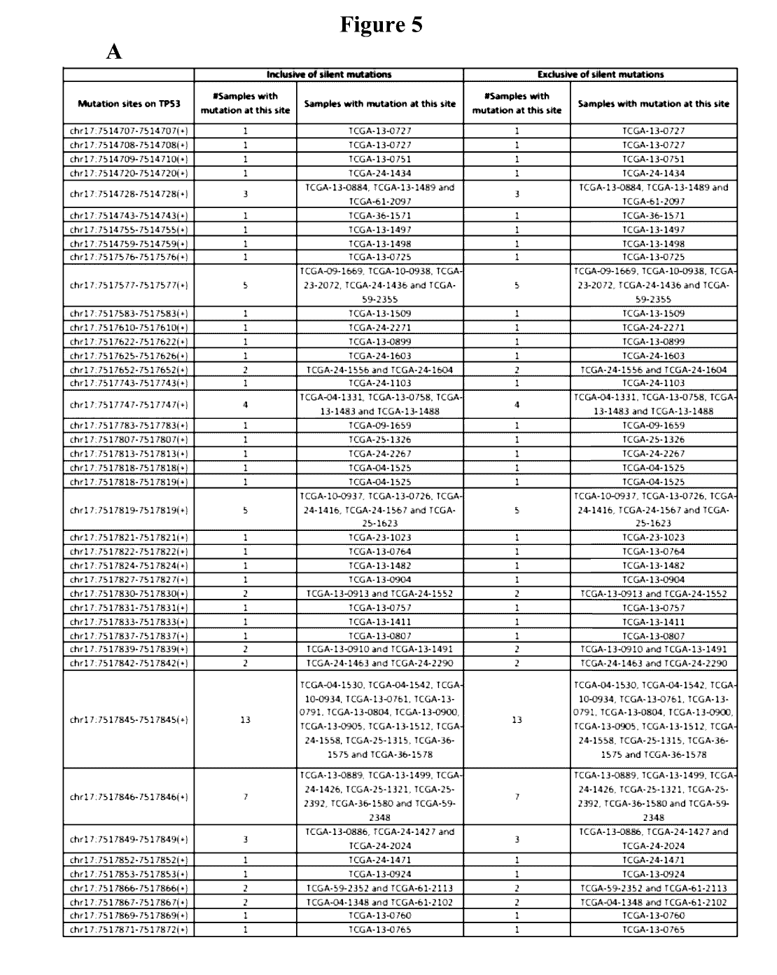

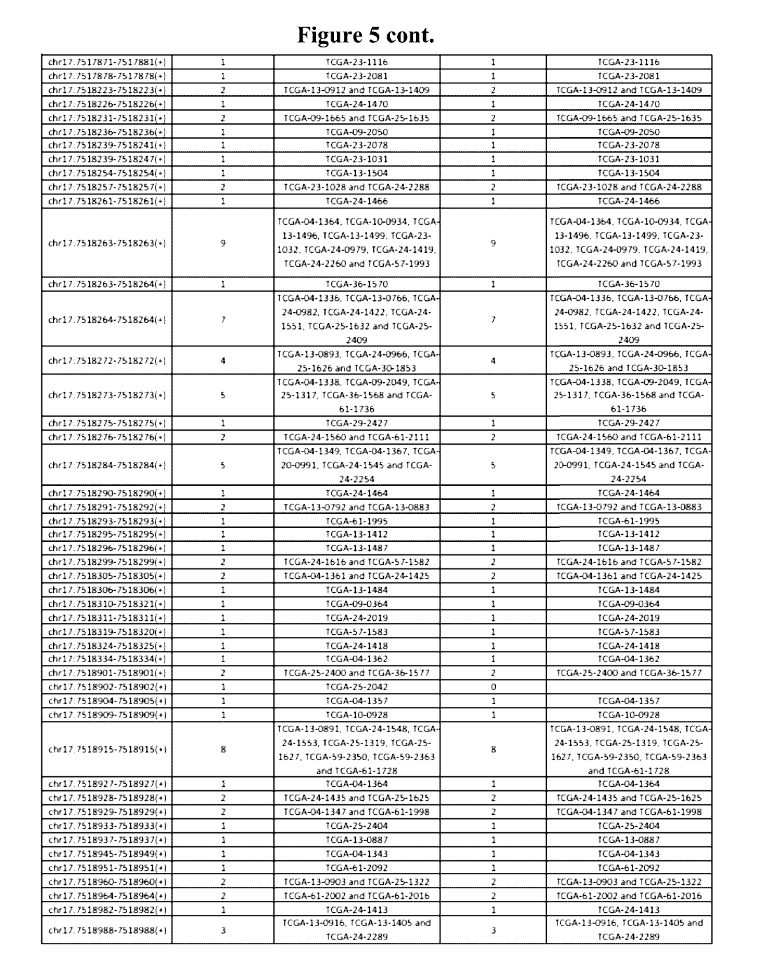

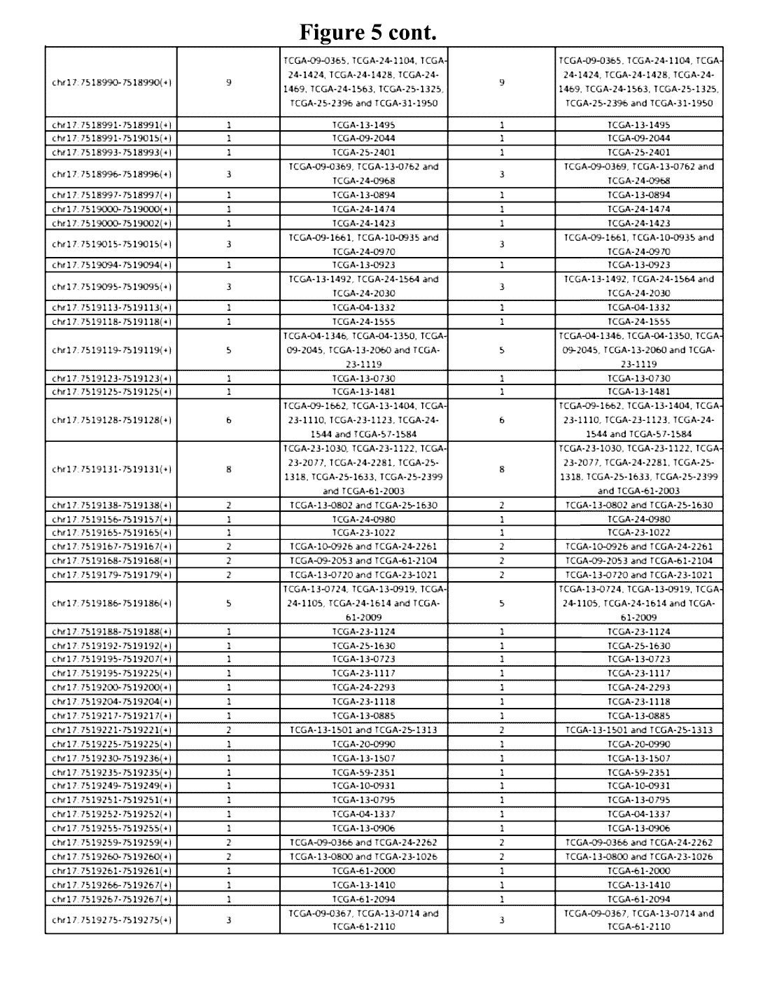

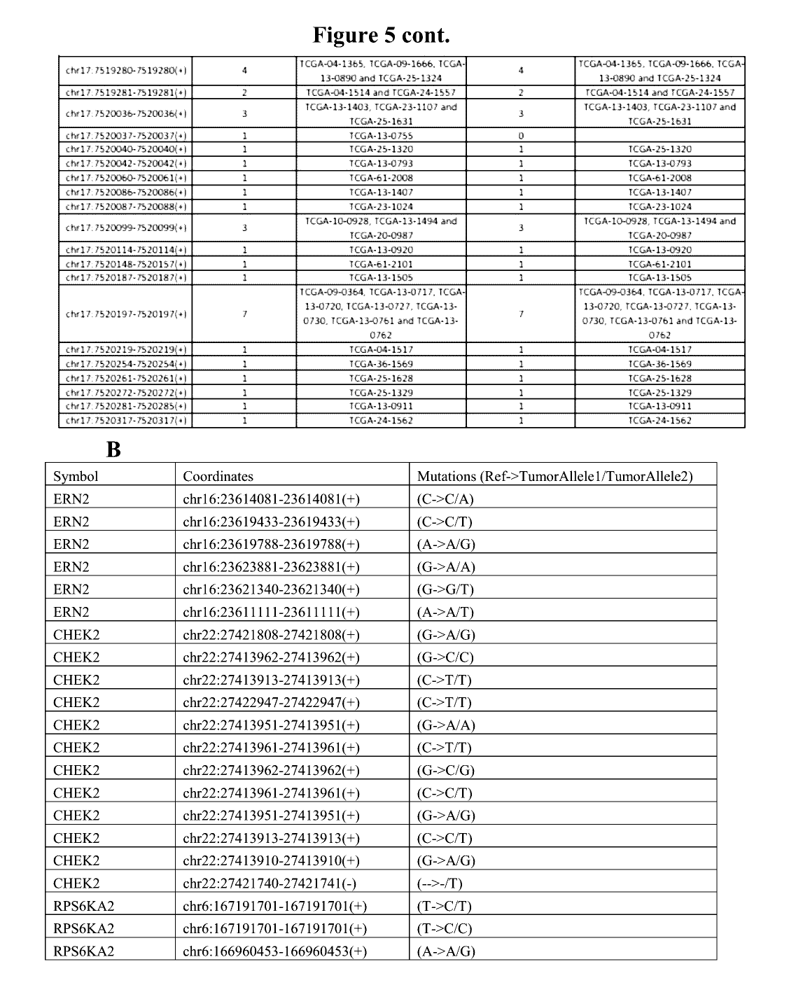

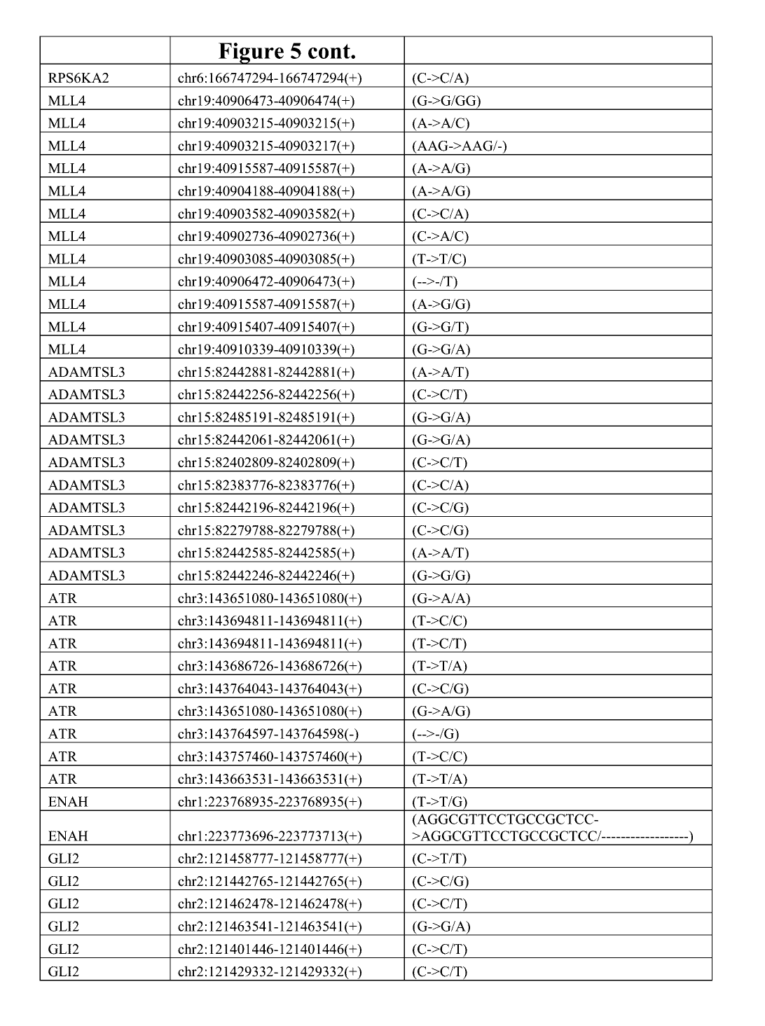

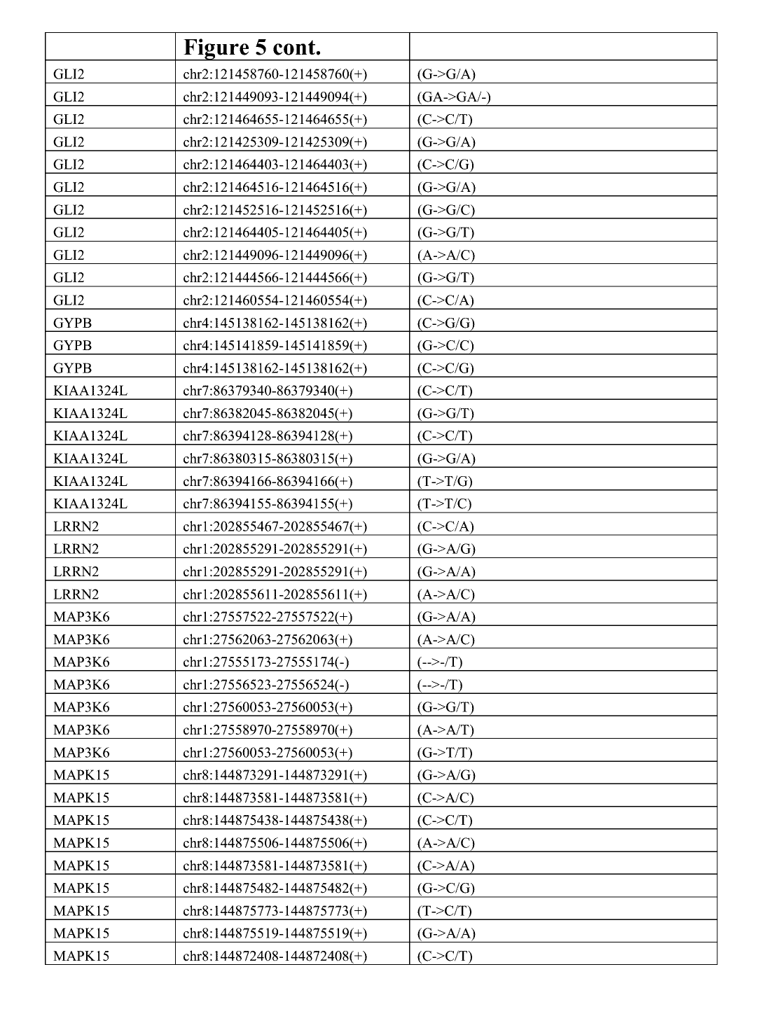

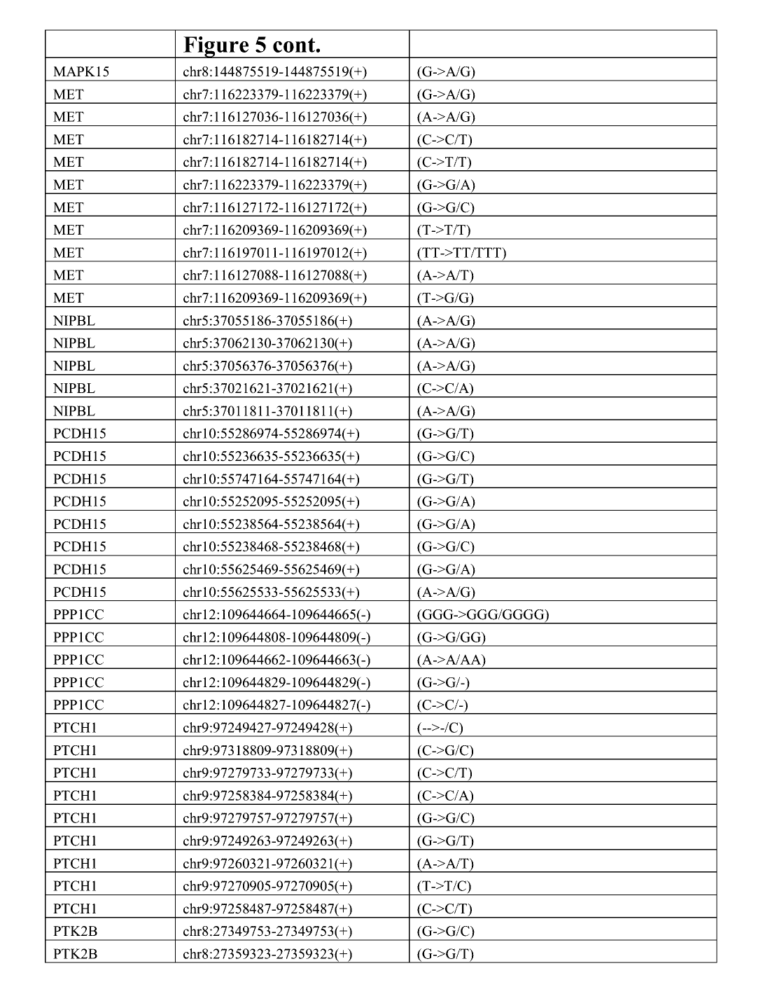

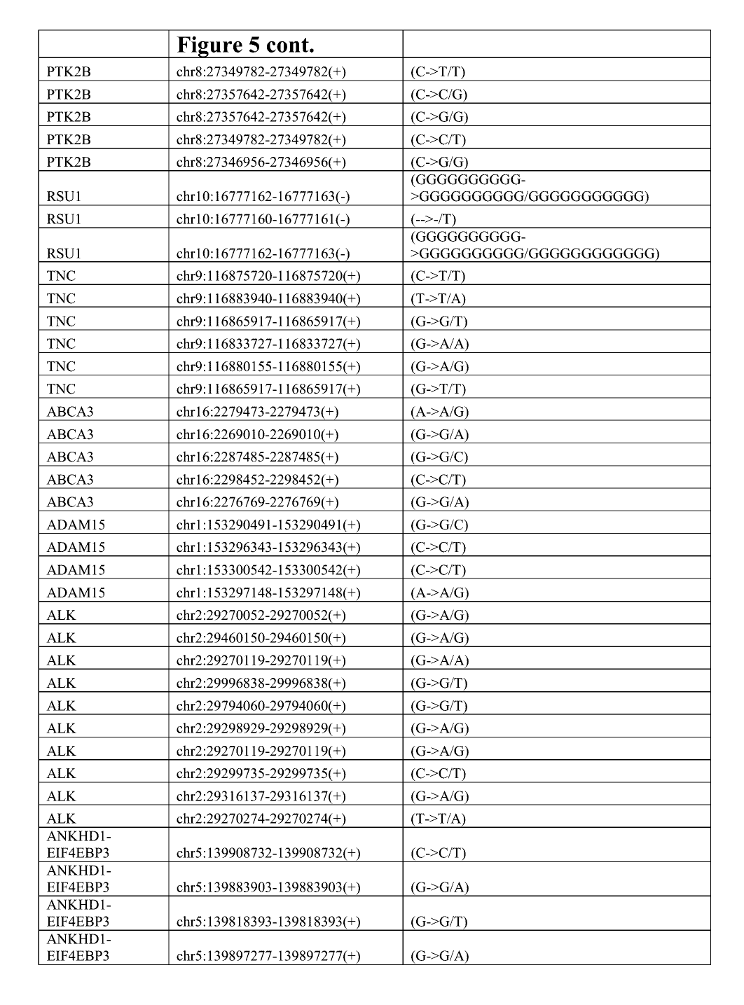

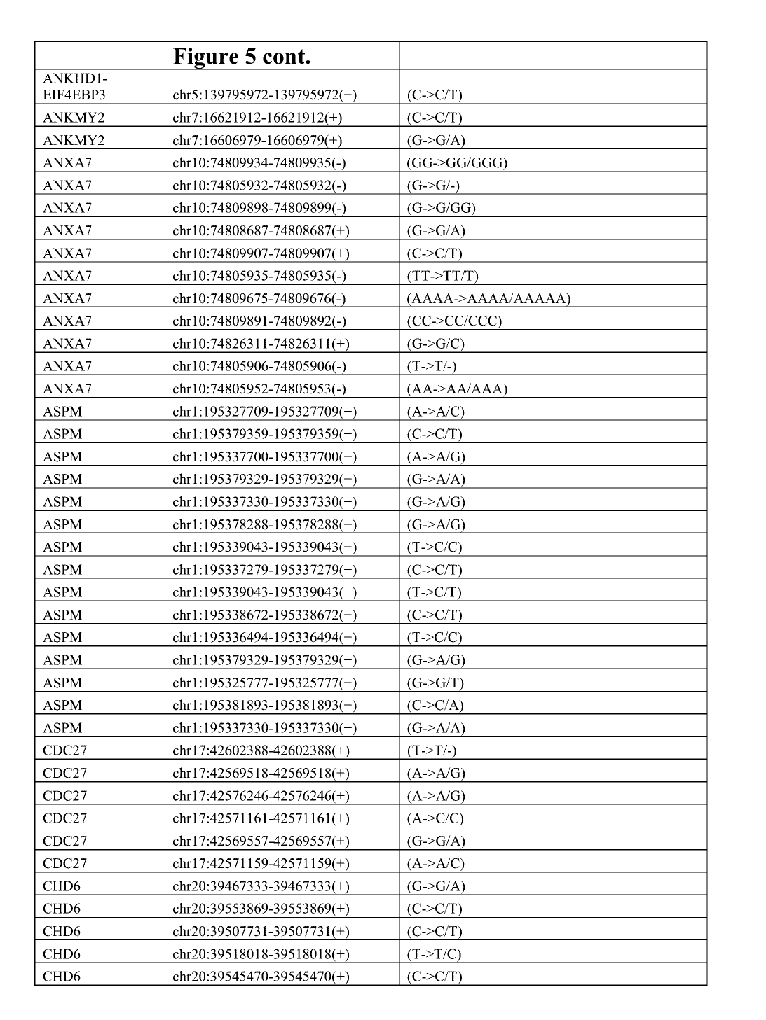

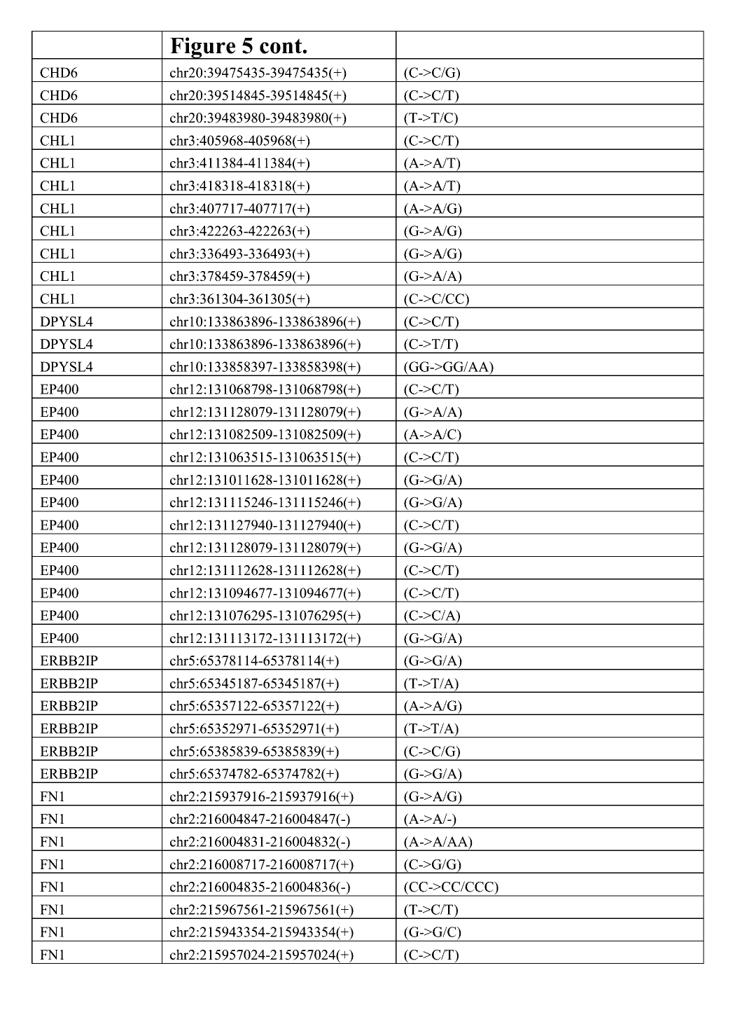

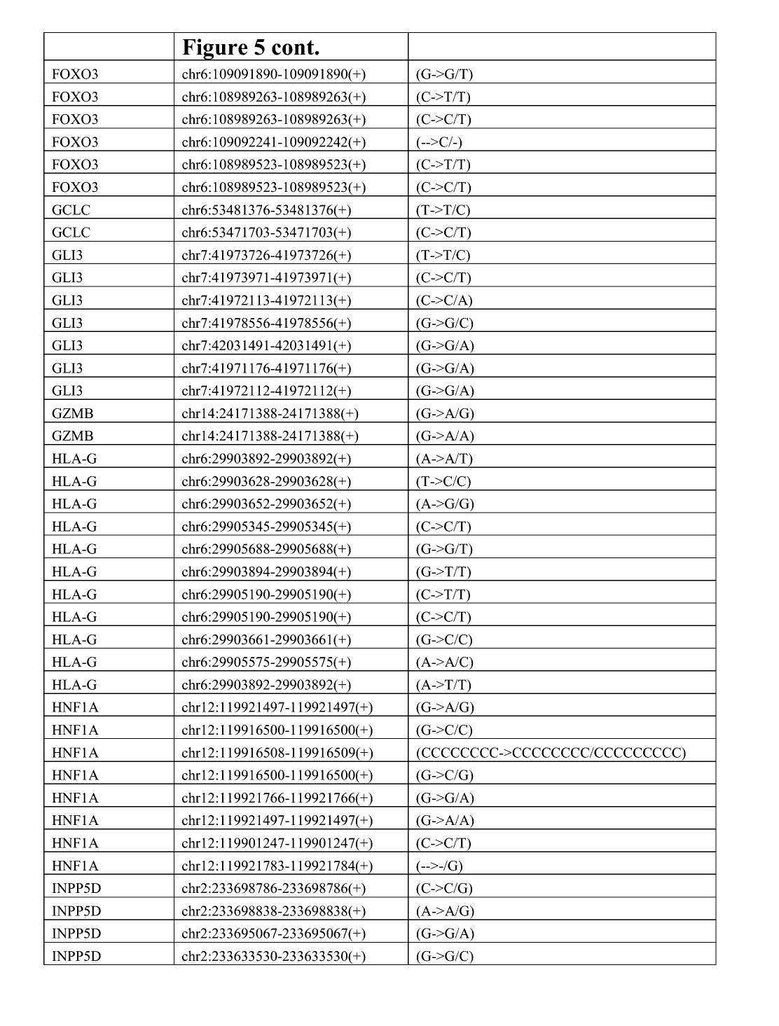

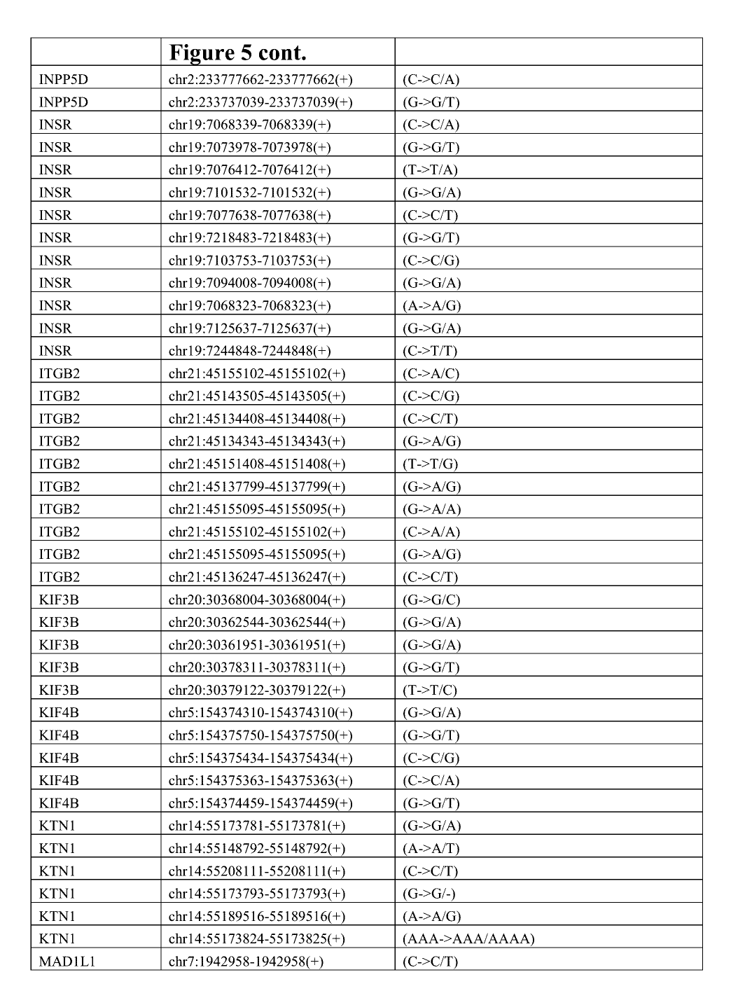

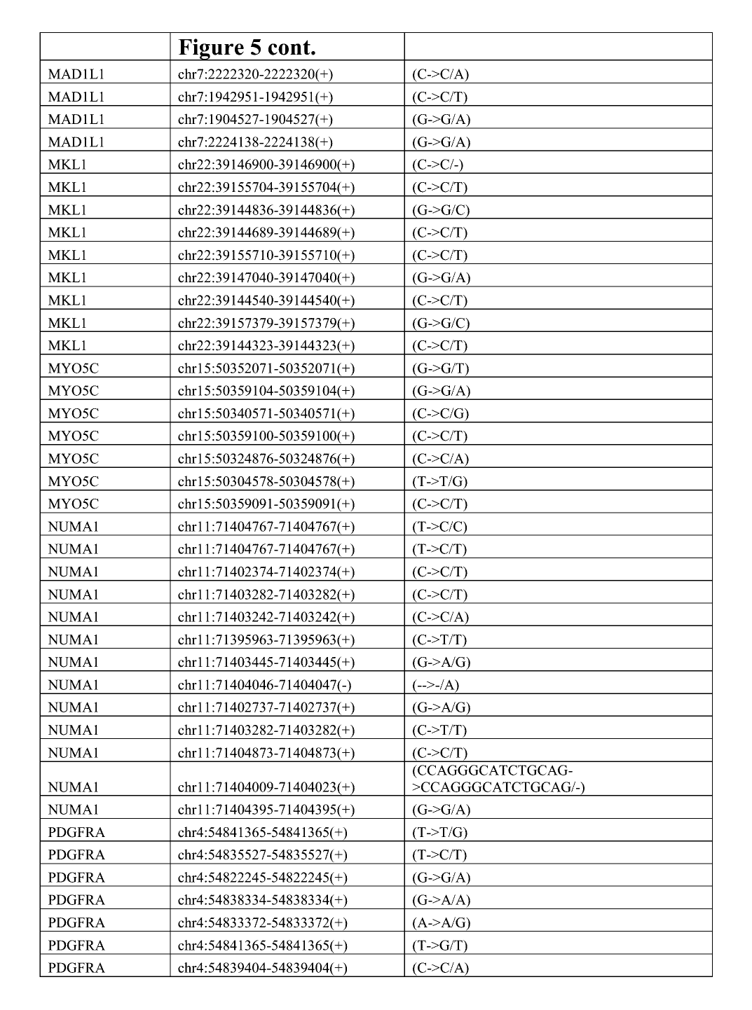

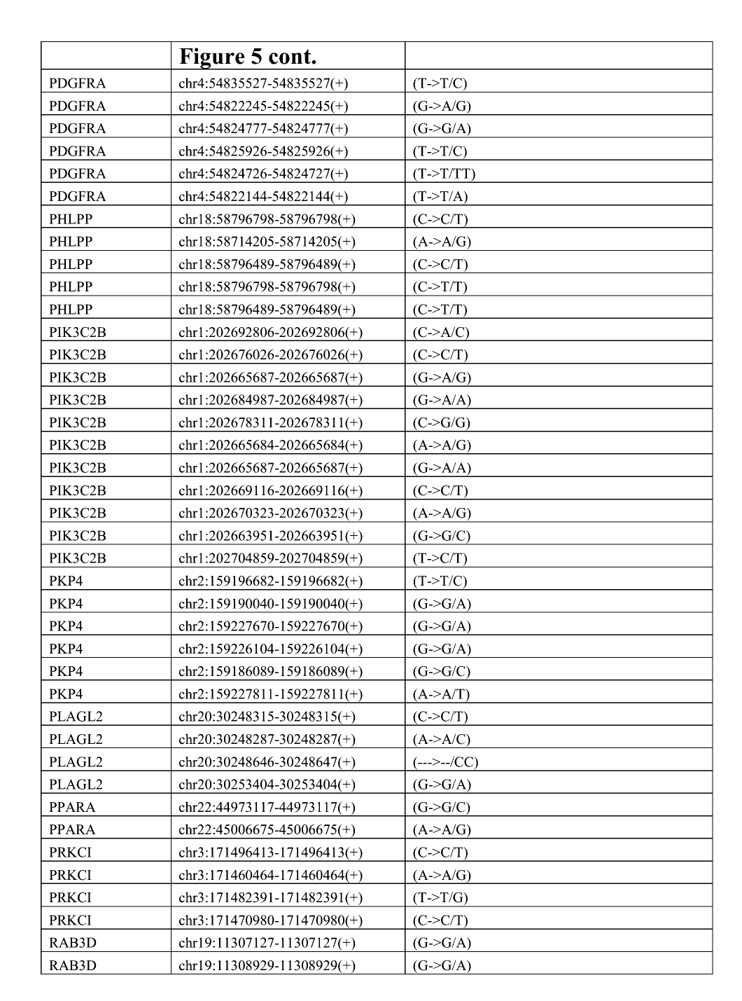

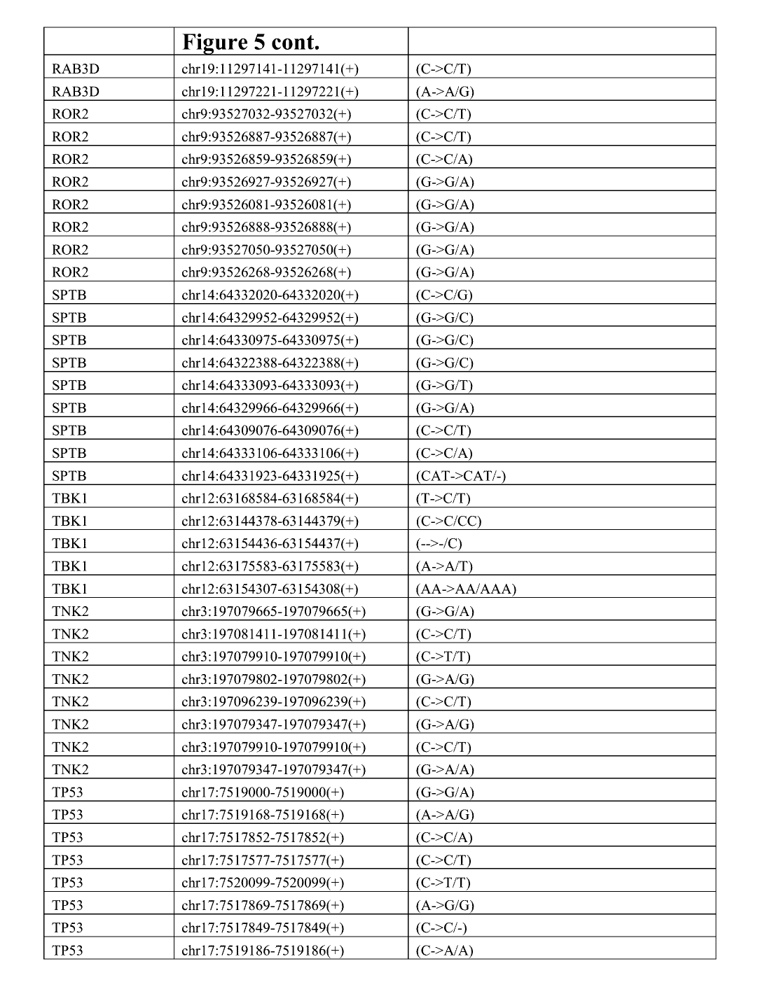

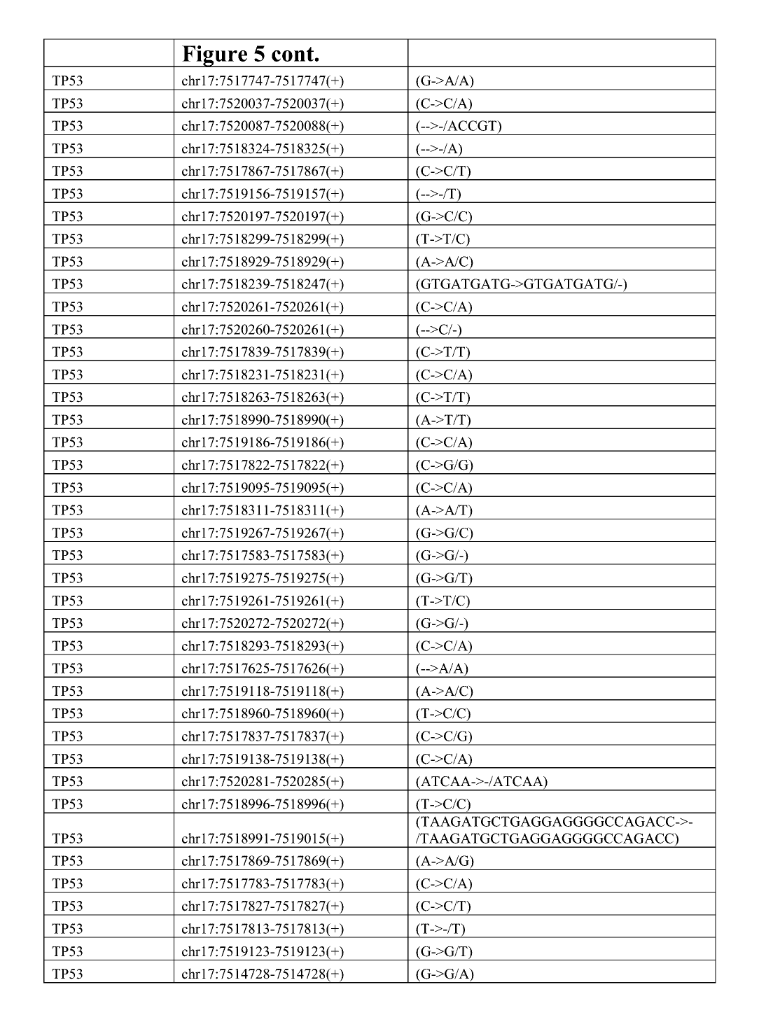

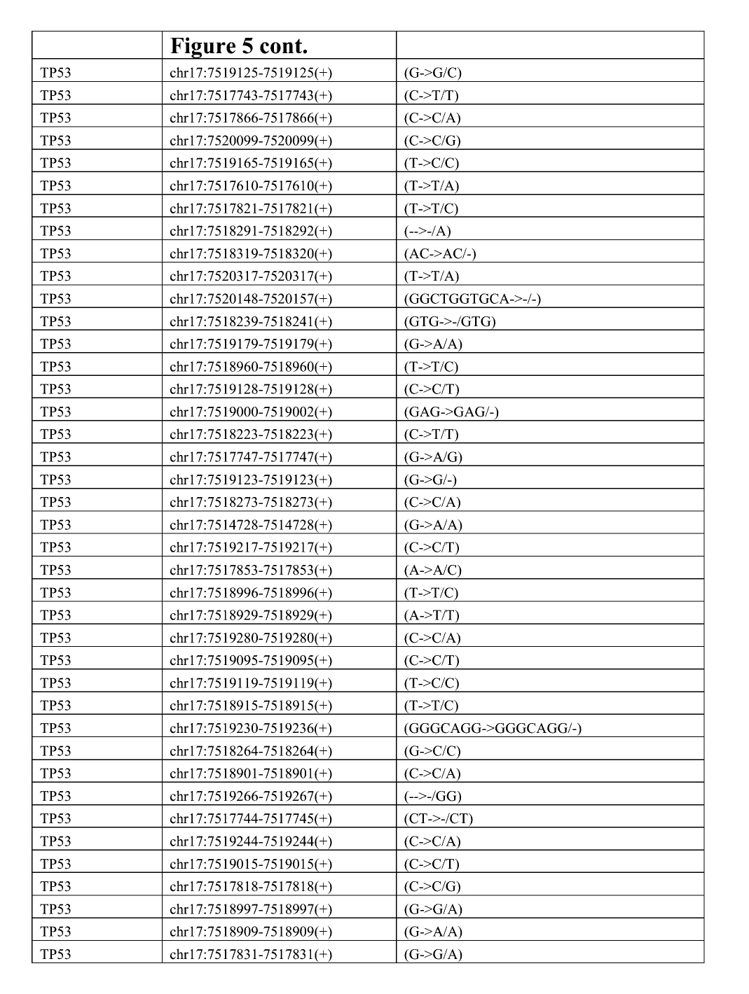

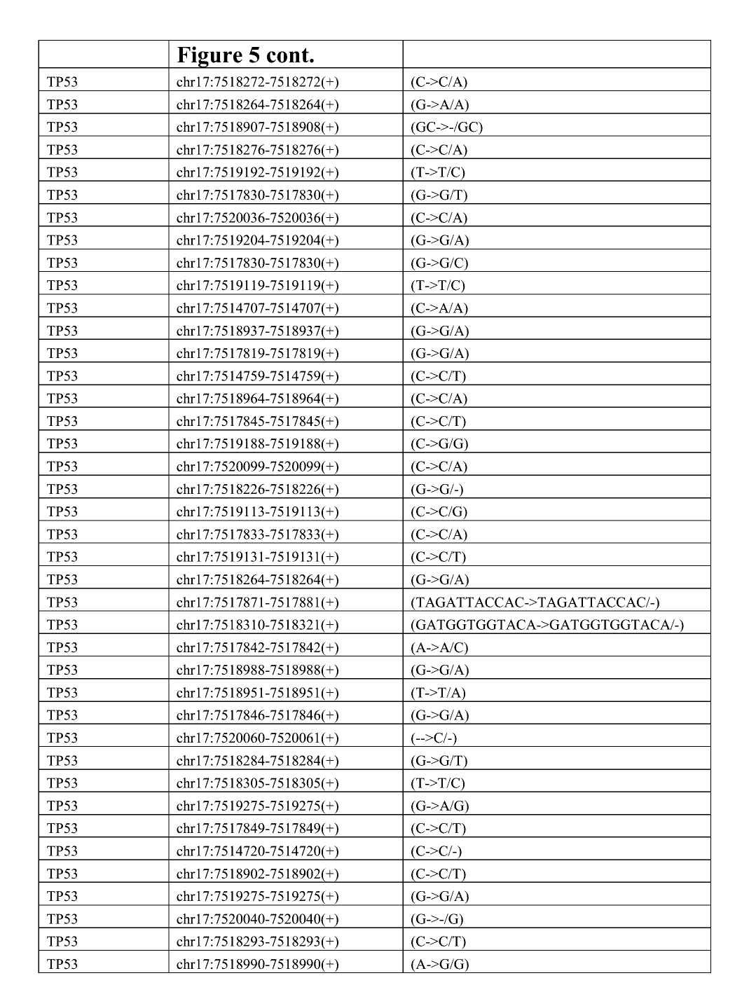

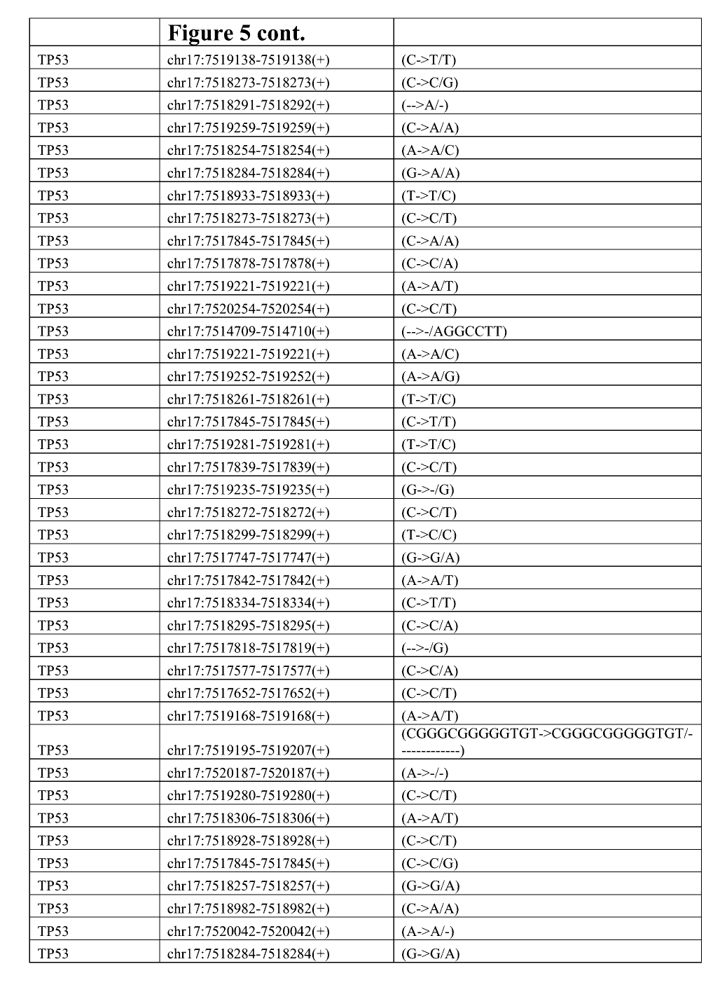

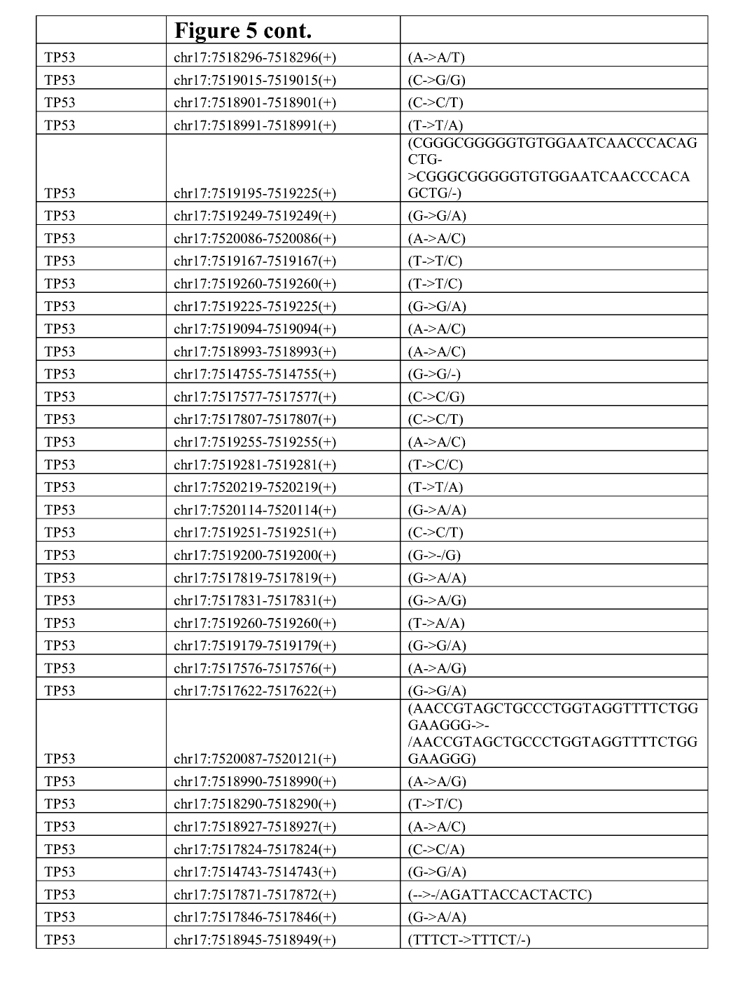

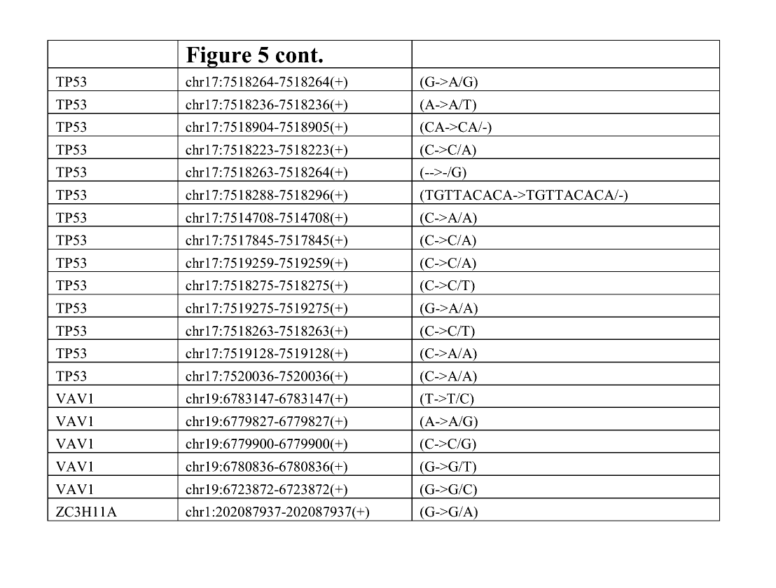

In various embodiments the mutations identified that can be used for the prognosis are listed in FIG. 5 in relation to their chromosome site location. A person skilled in the art can easily obtain the specific mutation based on this information using standard software such as d-chip software or others available.

Another aspect of the invention relates to a kit for carrying out the method described herein, the kit comprising at least one detection reagent capable of detecting a mutation, such as a nucleic acid probe complementary to wildtype or mutated mRNA or primers that allow amplification and then sequencing of the amplified sequences or primers that allow direct sequencing, in any one of the ABCA3, ADAM15, ADAMTSL3, ALK, ANKHD1-EIF4EBP3, ANKMY2, ANXA7, ASPM, CDC27, CHD6, CHL1, DPY5L4, ENAH, EP400, ERBB2IP, FN1, FOXO3, GCLC, GLI2, GLI3, GYPB, GZMB, HLA-G, HNF1A, INPP5D, INSR, ITGB2, KIF3B, KIF4B, KTN1, LRRN2, MAD1L1, MAP3K6, MAPK15, MET, MKL1, MLL4, MYO5C, NUMA1, PDGFRA, PHLPP, PIK3C2B, PKP4, PLAGL2, PPARA, PRKCI, PTK2B, RAB3D, ROR2, RPS6KA2, RSU1, SPTB, TBK1, TNK2, TP53, VAV1 and ZC3H11A marker genes.

The mutations in said marker genes are those that have been described above in relation to the inventive methods and and detection of them can be made by standard sequencing methods.

In various embodiments the detection reagent is a nucleic acid probe being complementary to wildtype mRNA of any one of the sequences set forth in SEQ ID NOS. 1-21 and 67-102.

In various embodiments, the kit comprises at least one nucleic acid probe complementary to mRNA of any one of ABCA3, ADAM15, ADAMTSL3, ALK, ANKHD1-EIF4EBP3, ANKMY2, ANXA7, ASPM, CDC27, CHD6, CHL1, DPYSL4, ENAH, EP400, ERBB2IP, FN1, FOXO3, GCLC, GLI2, GLI3, GYPB, GZMB, HLA-G, HNF1A, INPP5D, INSR, ITGB2, KIF3B, KIF4B, KTN1, LRRN2, MAD1L1, MAP3K6, MAPK15, MET, MKL1, MLL4, MYO5C, NUMA1, PDGFRA, PHLPP, PIK3C2B, PKP4, PLAGL2, PPARA, PRKCI, PTK2B, RAB3D, ROR2, RPS6KA2, RSU1, SPTB, TBK1, TNK2, TP53, VAV1 and ZC3H11A marker genes.

In various embodiments, the kit comprises a panel of nucleic acid probes complementary to mRNA of ADAMTSL3, ATR, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC marker genes.

In various embodiments, the kit comprises at least one nucleic acid probe complementary to mRNA of any one of marker gene sequences set forth in SEQ ID NOS 1-175, and optionally written instructions for: extracting nucleic acid from the sample of the patient and hybridizing the nucleic acid to a DNA microarray; and obtaining the prognosis of overall survival or prediction of therapeutic outcome for the patient.

In various embodiments the kit comprises a panel of nucleic acid probes complementary to mRNA of the marker gene sequences as set forth in SEQ ID NOS 1-21, 67-102.

In various embodiments, the kit further comprises at least one nucleic acid probe complementary to mRNA of any one of the marker gene sequences as set forth in SEQ ID NOS 3-5, 7-9, 11-14, 19, 20, 22-73, 77-79 and 99-175.

In various embodiments, the probes are able to detect mutations in the markers. This may be achieved using probes that are complementary to the markers whereby when they are used with a PCR melt technique the hybridization affinity between the mutant and the probe is less than the hybridization affinity between the standard nucleic acid and the probe at higher temperatures whereby a mutation can be identified. Alternative probes may include the mutation, otherwise being substantially complementary to the wildtype mRNA of any one of marker sequences.

In various embodiments, the kit comprises a panel of nucleic acid probes complementary to mRNA of ABCA3, ADAM15, ADAMTSL3, ALK, ANKHD1-EIF4EBP3, ANKMY2, ANXA7, ASPM, CDC27, CHD6, CHL1, DPYSL4, ENAH, EP400, ERBB2IP, FN1, FOXO3, GCLC, GLI2, GLI3, GYPB, GZMB, HLA-G, HNF1A, INPP5D, INSR, ITGB2, KIF3B, KIF4B, KTN1, LRRN2, MAD1L1, MAP3K6, MAPK15, MET, MKL1, MLL4, MYO5C, NUMA1, PDGFRA, PHLPP, PIK3C2B, PKP4, PLAGL2, PPARA, PRKCI, PTK2B, RAB3D, ROR2, RPS6KA2, RSU1, SPTB, TBK1, TNK2, TP53, VAV1 and ZC3H11A marker genes.

In various embodiments, the kit comprising a panel of nucleic acid probes complementary to mRNA of marker sequences as set forth in SEQ ID NOS 1, 3-5, 7-9, 11-14 19, 20 and 22-69.

Another aspect of the invention relates to a method for predicting the risk of a patient developing high-grade serous ovarian cancer (HG-SOC) comprising determining the presence or absence of a germ line mutation in a gene selected from CHEK2, RPS6KA2 and MLL4 in a sample obtained from said patient, wherein the presence of a mutation in the CHEK2, RPS6KA2 and/or MLL4 gene is indicative of the patient developing HG-SOC.

In various embodiments of the invention, the mutation in the one or more marker genes is detected by analyzing a sample obtained from the patient. The sample typically contains nucleic acid, and may, for example, be a body fluid, cell or tissue sample. Body fluids comprise, but are not limited to blood, blood plasma, blood serum, cerebrospinal fluid, cerumen (earwax), endolymph and perilymph, gastric juice, mucus (including nasal drainage and phlegm), peritoneal fluid, pleural fluid, saliva, sebum (skin oil), semen, sweat, tears, vaginal secretion, nipple aspirate fluid, vomit and urine. In certain embodiments of the methods detailed above, the body fluid is selected from the group consisting of blood, serum, plasma, urine, and saliva. The tissue sample may be ovary tissue and the cell sample may comprise cells from ovary or fallopian tissue.

The present technology also encompasses the use of germline mutations of CHEK2 and/or RPS6KA2 and/or MLL4 genes (DNA and/or mRNA and/or protein) as risk factors in predicting healthy women's risk of HG-SOC initiation and development.

In various embodiments, the germ line mutation is indicative for an increased risk of said patient developing HG-SOC.

In various embodiments the germ line mutations identified that can be used for the diagnosis are listed in FIG. 23.

As mentioned herein the methods of diagnosis may improve efforts to identify women at high risk of the hereditary and somatic mutations of ovarian cancers distinct from those which are associated with p53 somatic mutations and germline BRAC1/BRAC2 mutations.

Another aspect of the invention relates to a kit for carrying out the method of diagnosis, the kit comprising at least one nucleic acid probe complementary to mRNA of any one of CHEK2, RPS6KA2 and MLL4 markers

In various embodiments the nucleic acid probe complementary to mRNA comprises marker sequences complementary to mRNA any one of nucleic acid sequences set forth in SEQ ID NOS 1, 3, and/or 4 and optionally written instructions for: extracting nucleic acid from the sample of the patient and hybridizing the nucleic acid to a DNA microarray; and obtaining the risk of said patient developing HG-SOC.

It should be understood that all embodiments disclosed above in relation to the methods or uses of the invention, are similarly applicable to each method and use and vice versa.

As already described above, the importance of biomarkers and the technical advantage of the quantitative method holds great promise for understanding the etiology, pathophysiology, and more importantly, prognosis and diagnosis of subjects afflicted by HG-SOC particularly, with respect to patient survival events and times.

The present technology includes methods that (i) identify mutation of CHEK2 gene (DNA and/or mRNA and/or protein) as an important risk and poor prognostic factor for patients with HG-SOC, (ii) identify a combined mutational signature comprising 58 relatively often mutated genes in 7% of HG-SOC, that identify HG-SOC patients significantly associated with poor prognosis, (iii) identify a combined mutational signature comprising 21 genes (DNA and/or mRNA and/or protein) that significantly stratifies a cohort of patients into low and high-risk subgroups, and (iv) using either the CHEK2 gene (DNA and/or mRNA and/or protein) or the 58-gene signature (DNA and/or mRNA and/or protein) or the 21-gene signature (DNA and/or mRNA and/or protein) as an obligatory prognostic tool in the overall survival and treatment outcome prediction of individual HG-SOC patients in a clinical setting.

Genes comprised in the 58-gene and 21-gene mutational signatures are associated with functions such as kinase activity and ATP-binding, and they are also enriched in biological processes such as cell-cycle regulation, apoptotic control and DNA damage repair. The use of these gene mutational signatures significantly stratifies diagnosed HG-SOC patients into low and high-risk subgroups. Specifically, the 21-gene mutational signature provides stratification of the patients onto two disease development risk groups with their 5 year overall survival rates as 37% and 6%, respectively. Furthermore, the tumors in high-risk subgroup are about twice as likely to present resistance to therapy (15% of high-risk subgroup and 8.7% of low-risk subgroup).

CHEK2 mutations in HG-SOC patients are strong adverse indicator of patient survival prognosis and associated with therapy resistance. It is hypothesize without being limited to any theories that it could be due to mutations of the nuclear localization site which prevents the nuclear import of the protein and subsequently leads to haploinsufficiency. 21-gene mutational signature was also identified which highly correlates with patient's survival patterns (p=7.311e-08). Among these genes, protein functions such as kinase activity or ATP-binding are enriched, which possibly indicate that these processes play crucial roles in carcinogenesis and targeting these processes might be an attractive therapeutic strategy to restore the imbalance in dysregulated cell proliferation associated with the higher risk subgroups.

Two sub-classes of HG-SOC were characterized via either germline mutations of CHEK2, RPS6KA2 and MLL4 or somatic mutations of the other signature genes. The presence of a subset of tumors characterized via germline mutations or/and loss-of heterozygosity (LOH) of CHEK2 provides potential screening efforts to identify women with high-risk of developing HG-SOC.

Mutation counts across 9083 gene symbols and 334 tumor tissue samples from patients diagnosed with HG-SOC were analyzed. Expectedly, TP53 whose mutations are known to be one of the defining characteristic of HG-SOC, were found to be highly mutated across all samples. However, the frequency of TP53 mutations in each tumor sample was low, averaging about one TP53 per tumor sample. In contrast, CHEK2 and BRCA1 genes (which are involved in DNA damage repair) are mutated in high frequency in a small subset of patients.

Further unsupervised hierarchical clustering revealed a highly mutated cluster of 58 genes and 22 patients (from 334 HG-SOC) mainly characterized by CHEK2 mutations. Gene ontology and network analysis revealed that these genes are associated with kinase and ATP-binding, and could be involved in biological processes related to cell cycle, DNA damage repair, apoptosis or immune response. The cluster of 58 genes are: ABCA3, ADAM15, ADAMTSL3, ALK, ANKHD1-EIF4EBP3, ANKMY2, ANXA7, ASPM, CDC27, CHD6, CHEK2, CHL1, DPYSL4, ENAH, EP400, ERBB2IP, FN1, FOXO3, GCLC, GLI2, GLI3, GYPB, GZMB, HLA-G, HNF1A, INPP5D, INSR, ITGB2, KIF3B, KIF4B, KTN1, LRRN2, MAD1L1, MAP3K6, MAPK15, MET, MKL1, MLL4, MYO5C, NUMA1, PDGFRA, PHLPP, PIK3C2B, PKP4, PLAGL2, PPARA, PRKCI, PTK2B, RAB3D, ROR2, RPS6KA2, RSU1, SPTB, TBK1, TNK2, TP53, VAV1 and ZC3H11A.

The prognostic significance of the mutational status of the genes most highly mutated (in at least 5 patients) was assessed. 21 genes whose mutational status could independently and significantly stratify patients into low or high-risk (p-value≦0.05) were identified. These 21 genes are: ADAMTSL3, ATR, CHEK2, ENAH, ERN2, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC. Apart from ERN2 whose mutational status correlates with better overall survival, the other 20 genes have mutations that correlate with poorer overall survival. These genes are highly enriched in kinase activity and ATP-binding functions. They are also significantly enriched in pathways or gene networks of DNA damage and repair, apoptosis, and cell cycle.

A combined 21-gene mutational signature was subsequently composed where HG-SOC patients are classified into:

low-risk if mutations are observed in ERN2 and/or no mutations are observed in ADAMTSL3, ATR, CHEK2, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC.

high-risk if mutations are observed in ADAMTSL3, ATR, CHEK2, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC and/or mutations are not observed in ERN2.

Further analysis of the patients in the 58 high-risk patient subgroup defined by the 21-gene mutational signature revealed two distinct cluster of patients characterized by two different tumor subtypes. The first tumor subtype is characterized by germline mutations of CHEK2, RPS6KA2 and MLL4 whereas the second tumor subtype is characterized by spontaneous somatic mutations of the other genes. The results also revealed two possible disease etiology pathways in the presence of TP53 that typically characterize HG-SOC tumors. The screening of CHEK2, RPS6KA2 and MLL4 for genetic variants may be useful as risk factors in predicting a healthy woman's risk to developing the disease.

The gene mutational signatures can lend itself to potential applications in a clinical setting for early diagnosis of ovarian cancer as well as prognosis of therapy effectiveness and overall survival. The invention can also be used to identify subclass of tumors that are unlikely to respond well to chemotherapy and hence, provide scientists with a tool to develop new clinical strategies to target this tumor subclass. The gene mutational signatures can be formed into a diagnostic or prognostic kit to be used in the laboratory or in a clinical setting.

CHEK2 mutations were observed to be highly associated with poor response to chemotherapy and consequently, poor overall survival where 0% of high-grade serous ovarian carcinoma (HG-SOC) patients with CHEK2 mutations survive beyond 5 years. This subclass of patients with CHEK2 mutation constitutes about 7.2% of all HG-SOC patients. This allows identification of a previously unidentified subclass of patients diagnosed with high-grade serous ovarian carcinoma (HG-SOC). New lines of therapy or clinical management for this subclass of patients are urgently required as their tumors do not respond well to therapy. Therefore, this implies a necessity of identifying this subclass of HG-SOC patients, to provide better clinical care to this group of patient, and to study this subclass of tumors to derive future personalized clinical therapeutic benefit in future.

In addition to treating high grade and high stage serous ovarian cancer, detection of such cancer at the earliest stage could also be beneficial to the patient in terms of clinical intervention. Measuring the CHEK2 expression level to provide early diagnosis of ovarian cancer can help address such needs.

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples.

### Examples

### Genome-Wide Mutational Spectrum and Statistical Distribution of Gene Mutations

Exome sequencing via Illumina or ABI SOLID sequencing platforms was performed for 334 HG-SOC tumor samples at the Human Genome Sequencing Centers (HGSCs): Baylor College of Medicine (BCM), Broad Institute Genome Center (BI) and Genome Institute at Washington University (WUSM). The data was analysed by the TCGA research network as previously described (TCGA research network Nature 2011 Vol 474; 609-15). The processed mutational data was downloaded from the TCGA data portal for further analysis.

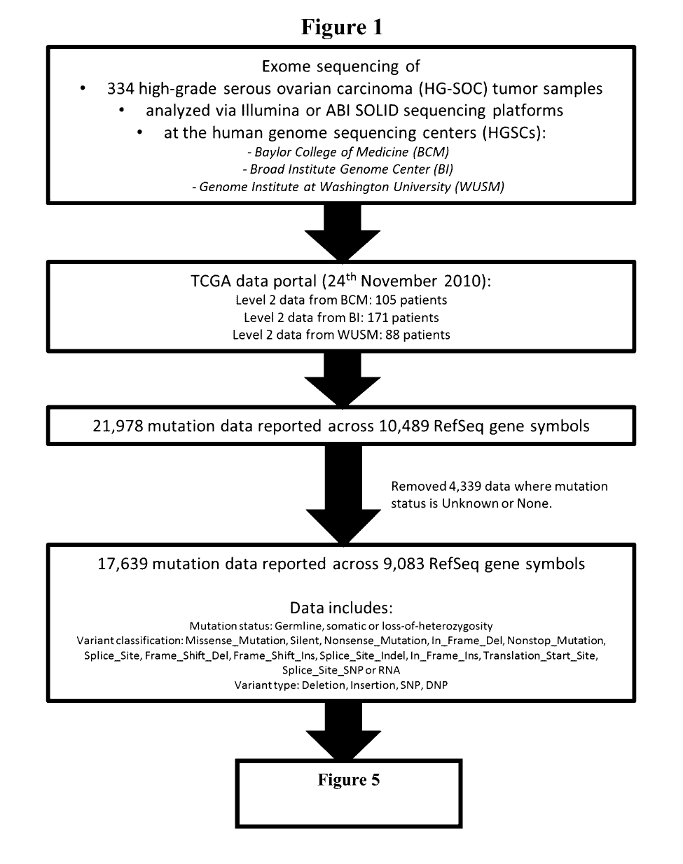

The TCGA data portal contains 21,978 mutations across all studied genes and patients. The genes whose mutational status was unknown were removed, and the remaining 17,639 mutations comprised of germline, LOH or somatic mutations across 334 patients and 9083 unique gene symbols (FIG. 1). These mutations encompassed all variants including deletion, insertion, missense mutations, or silent mutations of germline, somatic or loss-of heterozygosity (LOH) origins. The analysis included silent mutations, assuming that they could be conditional pathogenic mutations, specifically in context of DNA damage induced by common endogenous mutators (AID/APOBEC cytidine deaminases), regulatory signalling, modification of RNA-protein binding, post transcriptional events and cytosol-nuclear transport.

To provide genome-wide understanding of the relative frequency of occurrence of mutations within a gene and the relative frequency of gene mutations across the patient samples, A two-dimensional association matrix was first generated where the rows and columns correspond to 9083 unique gene symbols and 334 unique tumor sample IDs respectively (data not shown). The integer values in each cell of the matrix represent the number of unique mutation sites for each gene and each tumor sample ID. Frequency analysis of the mutated gene of this table demonstrated that all of the 23 mutated genes reported in previous TCGA studies were included in the subset of mutated genes, if two mutations are considered as a confidence threshold.

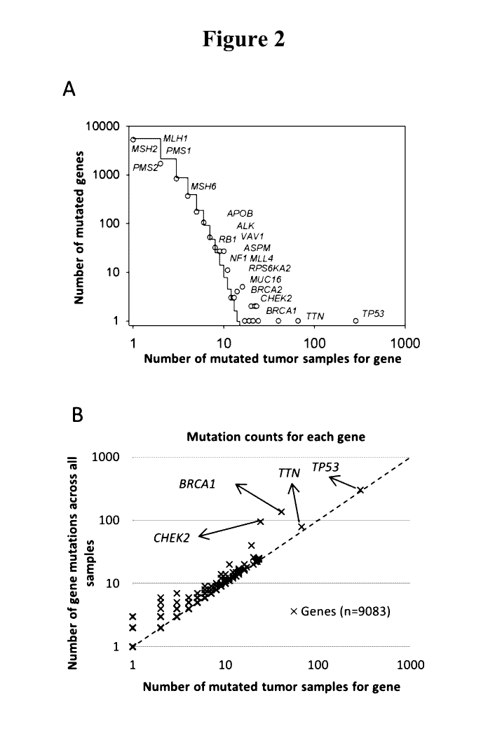

Subsequently for each gene, the number of tumor samples with reported mutation in this gene was calculated, as well as the total number of mutation events across all samples. The frequency distribution function of the number of tumor samples that are distributed across studied individual genes (N=9083) is shown in FIG. 2A. This figure shows examples of mutated genes with low, moderate and high frequency in the HG-SOC samples. In particular, FIG. 2A shows the relative high frequencies of tumor samples having mutations in BRCA1 (40 of 334 tumor samples) or BRCA2 (23 of 334 tumor samples) genes in the TCGA patient cohort. In contrast, mutations in DNA mismatch repair genes MLH1, MSH2, MSH6, PMS1 and PMS2 occurred in much fewer patients (1, 1, 4, 2 and 1 out of 334 patients, respectively). These genes are commonly associated with Lynch syndrome and accounts for a subset of hereditary ovarian cancers.

The frequency distribution is skewed with a long right tail, representative of observations that few genes are highly mutated whereas many other genes are less mutated in HG-SOC tumor samples. Such probability function belongs to a family of skewed distributions, which are observed often in many evolving and interactive (interconnecting) systems in which the birth-death process are occurring and driving a system by evolution towards the complexity and self-organization (see Methods). In such models, the skewed form of the function is strongly population/sample size and scale-dependent. In the context of cancer driving mutations, Kolmogorov-Waring (KW) model allows us to better understand the nature of enormous variability and plasticity of mutation events and a role of common and rare mutations in cancer origin and its progression. In practical sense, K-W model allows estimation of a fraction of the mutated genes which could be observed if the numbers of mutated tumour samples are increased. In this case, the best-fitted K-W function yields the following parameters:

$$a = 3.944 ; b = 9.50 ; θ = 0.867 and Po = ( 1 - \frac{a}{b} ) = 0.31$$

Thus, the total number of susceptible target genes Nₛ could be estimated by the formula:

Nₛ=Nb/a=9083×9.5/3.944=21887 genes.

This result suggests that the expected number of potential target genes for mutagenesis would include the entire set of protein-coding genes in humans. As the data only revealed 9083 mutated genes, the discrepancies could be false negatives and could be improved via increasing sample sizes or improvement of technology

Also, a scatter plot was generated where each point represents each gene and the axes represent the number of patient tumor samples with at least one mutation in that gene against the number of total mutation sites for the gene across all samples (FIG. 2B). The diagonal line represents a hypothetical situation where each gene is mutated exactly once per sample, if any at all. Our results indicated that while TP53 is the most highly mutated gene and was observed in almost all HG-SOC patients, the number of mutations with the gene locus in each patient sample is relatively low, i.e. on average, only 1 TP53 mutation was observed for each patient (298 mutations across 285 HG-SOC patients). Altered or loss of function of this tumor suppressor appears to be critical for HG-SOC carcinogenesis.

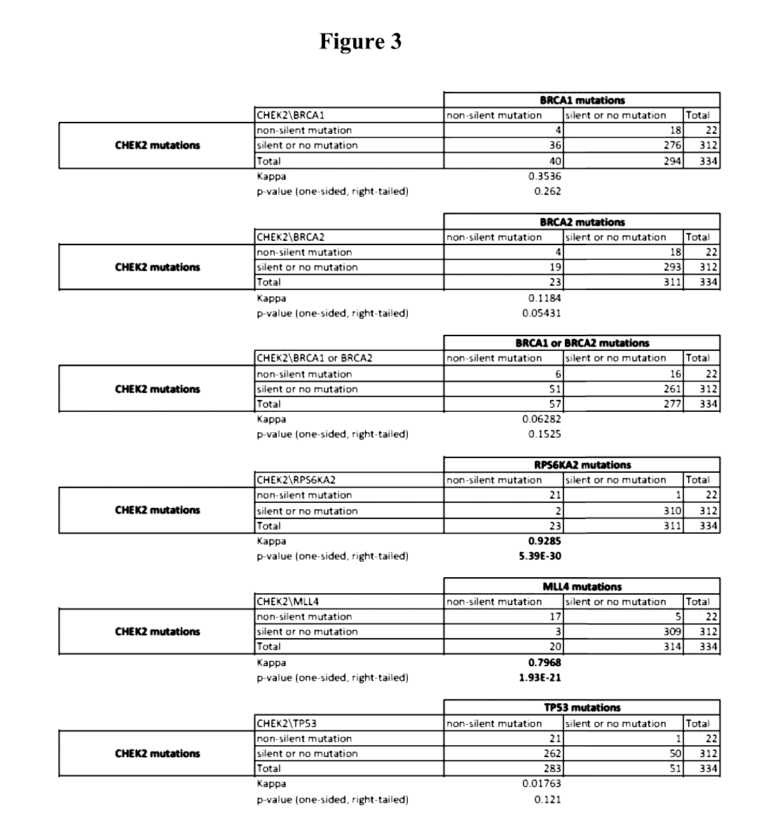

The other cancer susceptibility gene BRCA2 was less frequently mutated and only 25 mutations were observed in 23 HG-SOC mutations. CHEK2 and BRCA1 mutations appear to be mutually exclusive in HG-SOC patients, as only 18% (4 of 22) patients with non-silent CHEK2 mutations harbour BRCA1 mutations (FIG. 3). Similarly, only 18% (4 of 22) patients with non-silent CHEK2 mutations harbour BRCA2 mutations (FIG. 3).

### A Mutation Cluster is Defined by Genes Involved in Various Cell-Cycle Related Processes

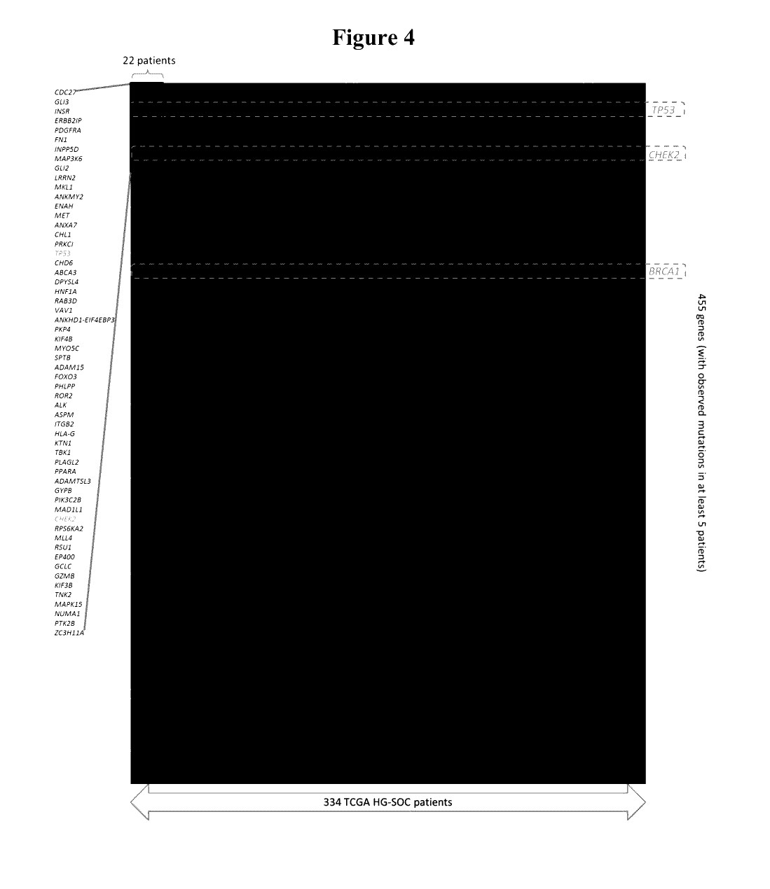

For a subset of 455 genes with observed mutations in at least 5 HG-SOC patients, unsupervised hierarchical clustering on the gene-patient mutation association matrix was performed. The full heatmap for 455 genes and 334 HG-SOC patients is shown in (FIG. 4). As expected, the TP53 mutations were observed in a majority of HG-SOC patients (85%, 285 out of 334). However, the intensity of TP53 mutations in a patient was low: generally only one TP53 mutation was observed in each p53 gene for a given patient. Interestingly, mutation sites along the TP53 locus appears to be randomly located across the exons and there appears to be no strong positive clonal selection for any particular gene variant (FIG. 5). The frequencies of mutations of other genes such as BRCA1 (12.0%, 40 out of 334) and CHEK2 (7.2%, 24 out of 334) across tumor samples were relatively smaller. However, the intensity of these mutations per gene is more than 3 times higher than for TP53 (on average, 3.38 and 3.96 mutations per patient for BRCA1 and CHEK2 respectively). The heatmap provides visual presentation of these finding. It also confirmed our previous finding that mutations in BRCA1 and in CHEK2 were generally mutually exclusive (FIGS. 3 and 4)

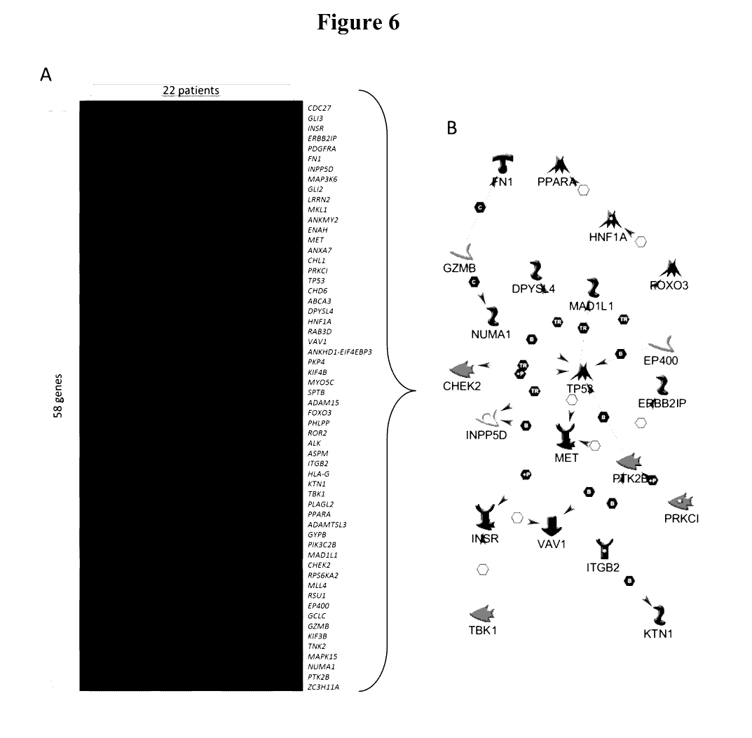

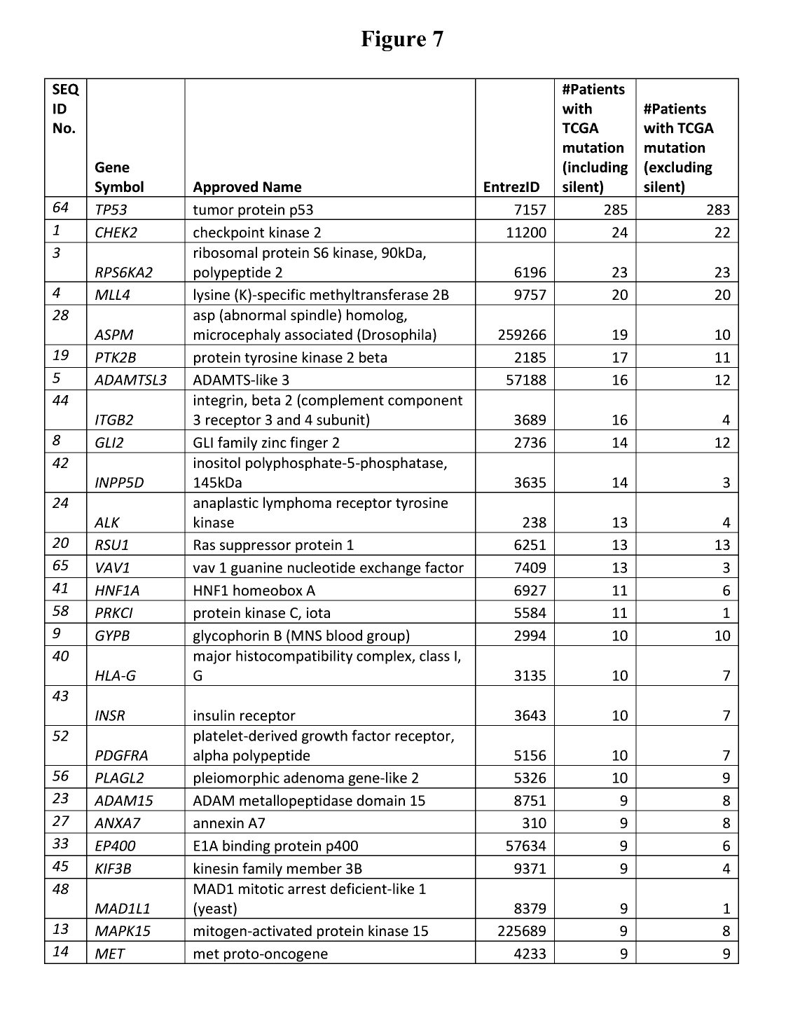

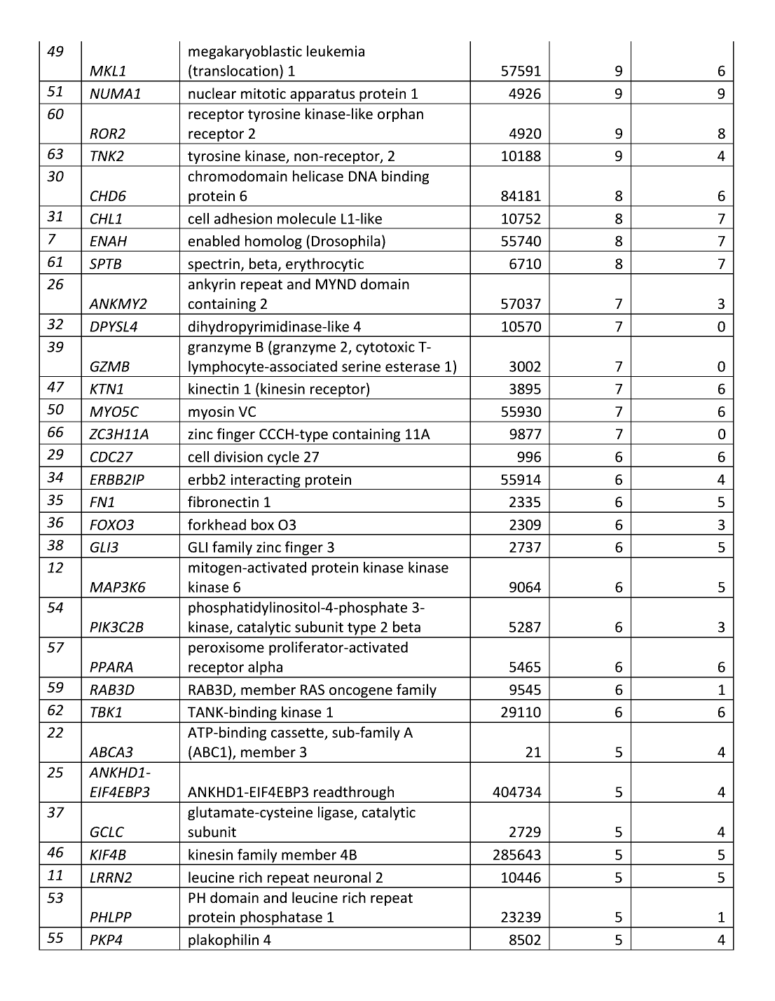

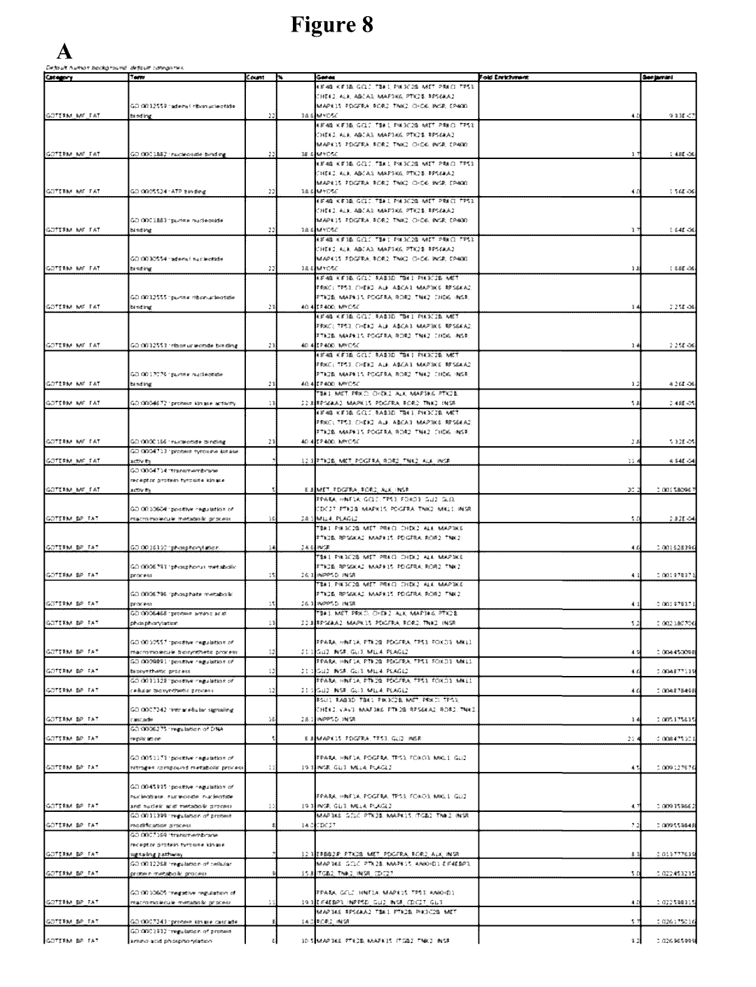

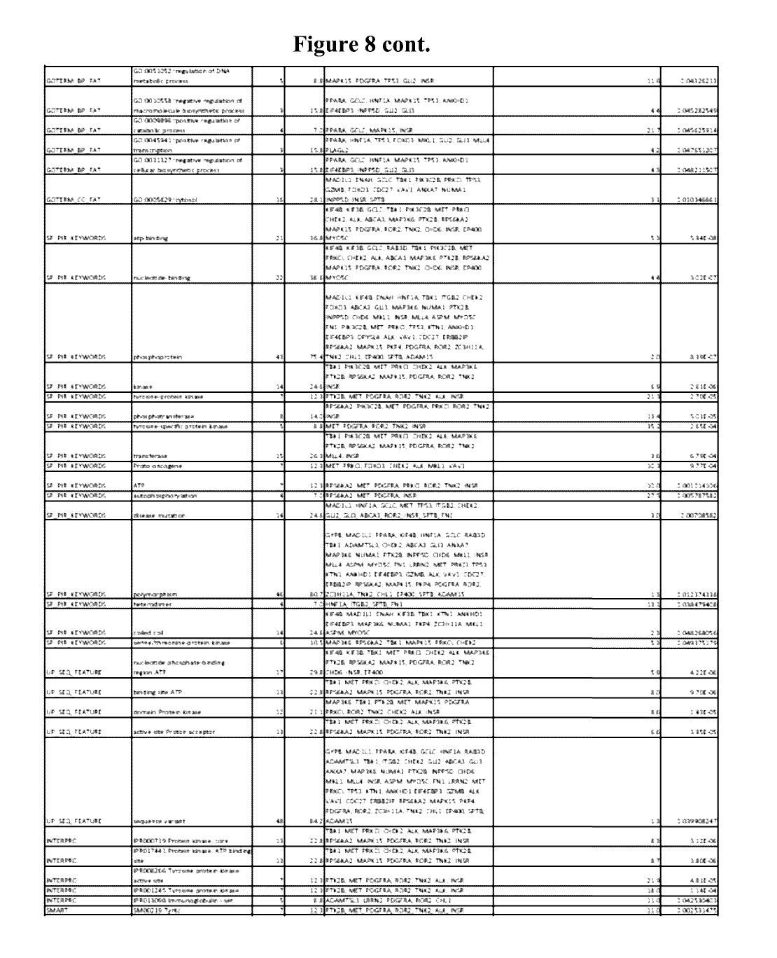

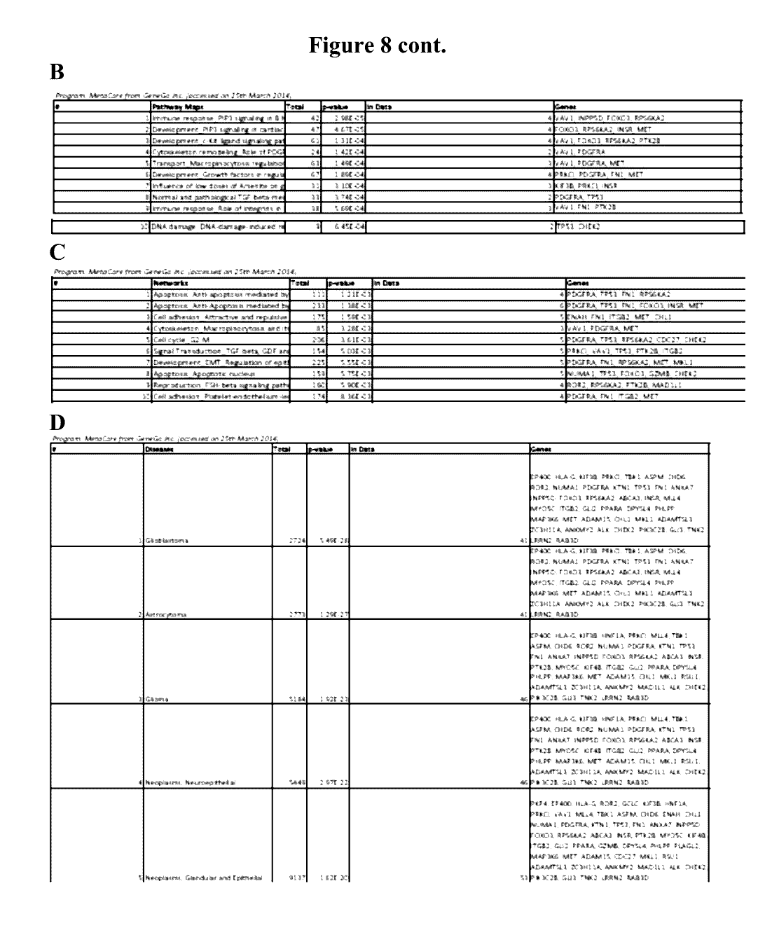

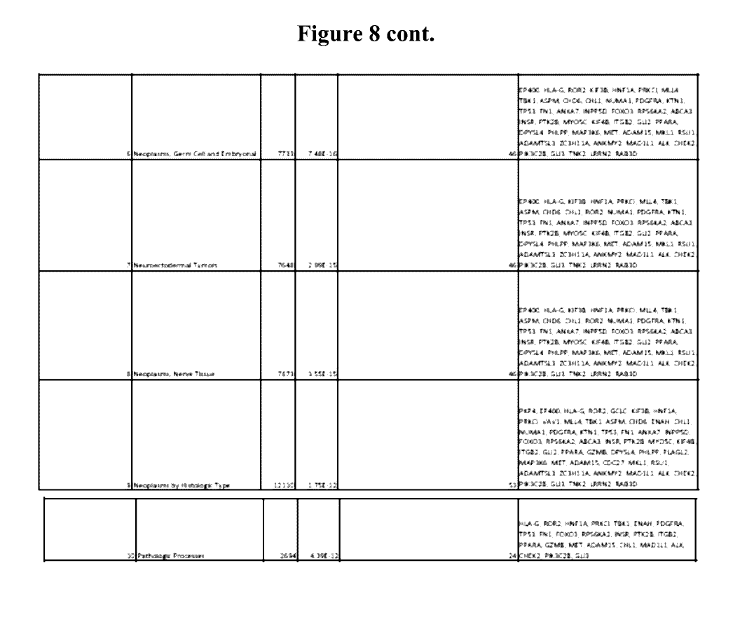

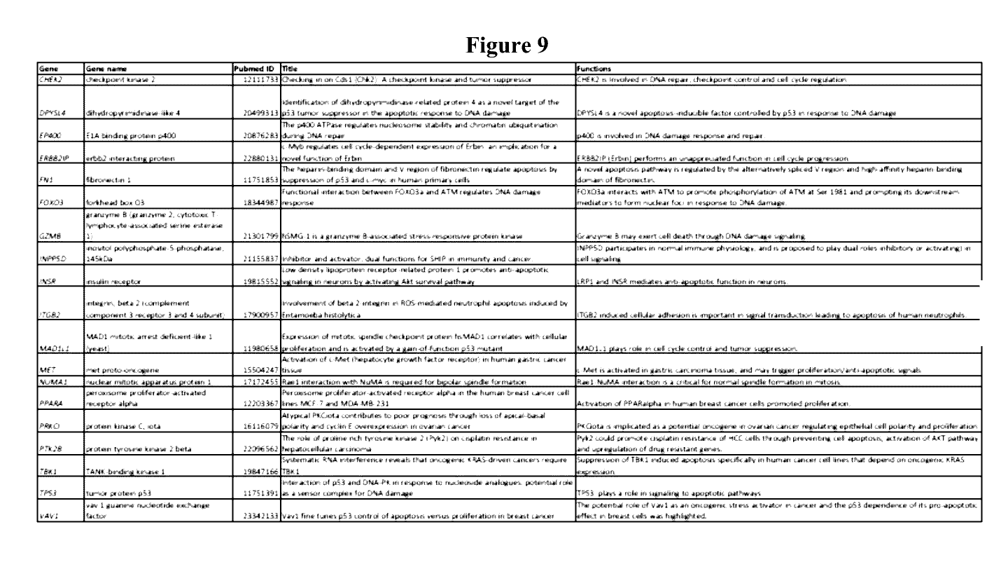

Results from hierarchical clustering also revealed a distinct gene-patient cluster associated 180 with CHEK2 (FIG. 4). This sub-cluster includes 58 gene symbols and 22 HG-SOC patients (FIG. 6). Within this cluster, mutations of CHEK2 appear to dominate, as multiple regions of CHEK2 were observed to be mutated in each of these patients (FIG. 6A). The annotation of the 58 gene symbols are listed in FIG. 7. Analysis of the 58 gene symbols via DAVID Bioinformatics revealed that these genes are significantly enriched in protein kinase activity (TBK1, PIK3C2B, MET, PRKCI, CHEK2, ALK, MAP3K6, PTK2B, RPS6KA2, MAPK15, PDGFRA, ROR2, TNK2 and INSR), adenyl and purine ribonucleotide binding (KIF4B, KIF3B, GCLC, TBK1, PIK3C2B, MET, PRKCI, TP53, CHEK2, ALK, ABCA3, MAP3K6, PTK2B, RPS6KA2, MAPK15, PDGFRA, ROR2, TNK2, CHD6, INSR, EP400 and MYO5C) and disease mutations (MAD1L1, HNF1A, GCLC, MET, TP53, ITGB2, CHEK2, GLI2, GLI3, ABCA3, ROR2, INSR, SPTB and FN1) (FIG. 8A). Further analysis via Metacore revealed significant association with immune response and DNA damage pathways as well as apoptotic and cell cycle gene networks (FIG. 8B,C). Network analysis of these 58 genes further identified a tight direct interacting network of 21 genes mostly involved in apoptosis, cell cycle control, DNA damage response and immune response (FIG. 6B). These biological categories and networks are strongly assigned to well-studied DNA damage, repair, cell cycle, check point regulation (FIG. 9).

The present technology describes a method of risk assessment, prognosis and therapy outcome prediction of high-grade serous ovarian carcinoma (HG-SOC) based on detection of germline and/or somatic mutations of DNA and/or mRNA and/or protein of the CHEK2-associated 58-mutated gene signature which comprises of:

ABCA3, ADAM15, ADAMTSL3, ALK, ANKHD1-EIF4EBP3, ANKMY2, ANXA7, ASPM, CDC27, CHD6, CHEK2, CHL1, DPYSL4, ENAH, EP400, ERBB2IP, FN1, FOXO3, GCLC, GLI2, GLI3, GYPB, GZMB, HLA-G, HNF1A, INPP5D, INSR, ITGB2, KIF3B, KIF4B, KTN1, LRRN2, MAD1L1, MAP3K6, MAPK15, MET, MKL1, MLL4, MYO5C, NUMA1, PDGFRA, PHLPP, PIK3C2B, PKP4, PLAGL2, PPARA, PRKCI, PTK2B, RAB3D, ROR2, RPS6KA2, RSU1, SPTB, TBK1, TNK2, TP53, VAV1 and ZC3H11A

and 21-mutated gene signature which comprises of:

ADAMTSL3, ATR, CHEK2, ENAH, ERN2, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC.

This present invention includes the methods, the resulting signature, and consequent clinical applications to prognosis of diagnosed HG-SOC patients or screening of healthy women for risk prediction of developing the disease.

The methods leading to the development of the CHEK2, 58-gene and 21-gene mutational signatures include:
the use of unsupervised hierarchical clustering and supervised statistical analysis to identify a highly mutated cluster of 58 genes and HG-SOC patients who are characterized by mutations of CHEK2.
unbiased screening of highly mutated genes in HG-SOC patients (at least 5 patients) to identify 21 prognostic genes (ADAMTSL3, ATR, CHEK2, ENAH, ERN2, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC) whose mutation status significantly and independently classify patients into low and high-risk subgroups.
the use of gene ontology, pathway and network analysis to assess and confirm the biological validity of 58 genes in the CHEK2-associated mutation cluster and 21 prognostic genes.
the use of Kaplan-Meier and log-rank test to confirm the prognostic significance of gene mutations in diagnosed HG-SOC patients.

The composition of a combined 21-gene mutational signature where:
HG-SOC patients are classified as low-risk if mutations are observed in ERN2 or no mutations are observed in ADAMTSL3, ATR, CHEK2, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC.
HG-SOC patients are classified as high-risk if mutations are not observed in ERN2 and mutations are observed in ADAMTSL3, ATR, CHEK2, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC.

The present technology proposes:
a method of using CHEK2 mutational status for overall survival prognosis and therapy response prediction for patients already diagnosed with HG-SOC.
a method of using the combined 58-gene mutational signature to identify high-risk subgroups associated mostly with germ-line or somatic mutations
a method of using the combined 21-gene mutational signature to classify patients into low or high-risk subgroup, where the 5-yr overall survival rates for the low and high-risk subgroups are 37% and 6%, respectively.
a method of using the combined 21-gene mutational signature to classify the high-risk patient subgroup into two further tumor subtypes based on characterization of germline and/or LOH and/or somatic mutations of genes of the 21-gene mutational signature. The first tumor subtype is associated with germline and/or LOH mutations of CHEK2 and/or RPS6KA2 and/or MLL4 genes whereas the other tumor subtype is associated with somatic mutations of other genes.
a method of using germline mutations of CHEK2 and/or RPS6KA2 and/or MLL4 genes to identify healthy women who might be susceptible to initiation, development and progression of tumors leading to HG-SOC.
   CHEK2 Mutations are Associated with Poor Prognosis of Diagnosed HG-SOC Patients

The initial analyses of the mutational spectrum of patients diagnosed with HG-SOC revealed a distinct gene-patient cluster where CHEK2 mutations appear to be highly concentrated in a few patients. Focusing subsequent analysis on CHEK2, mutations in this gene were examined to determine if they were associated with patient overall survival times, and if it could be used as a prognostic survival factor for patients already diagnosed with HG-SOC.

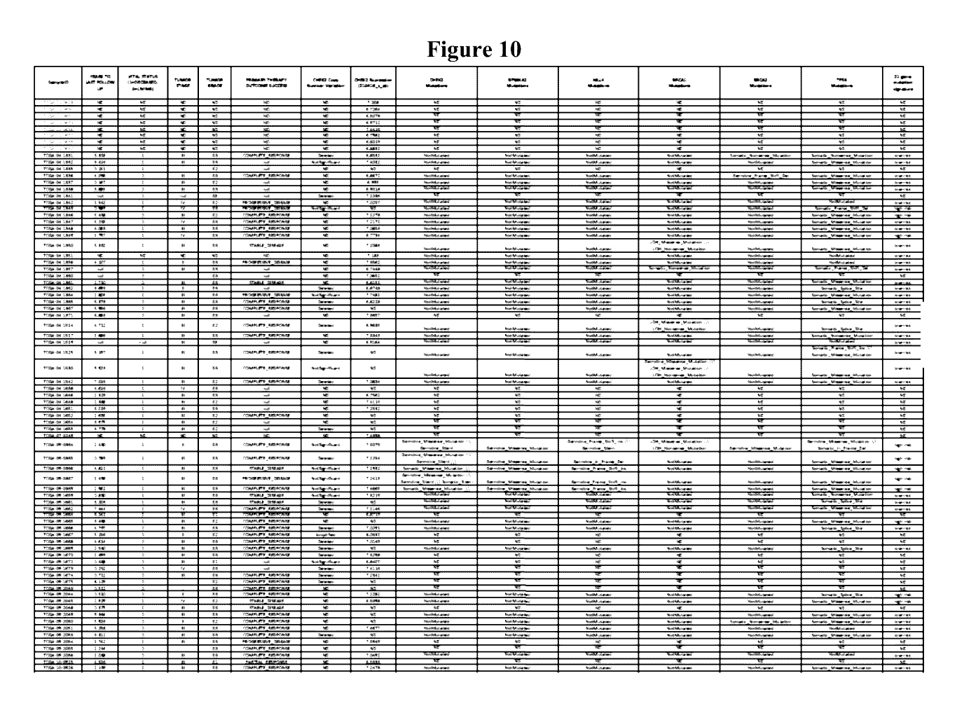

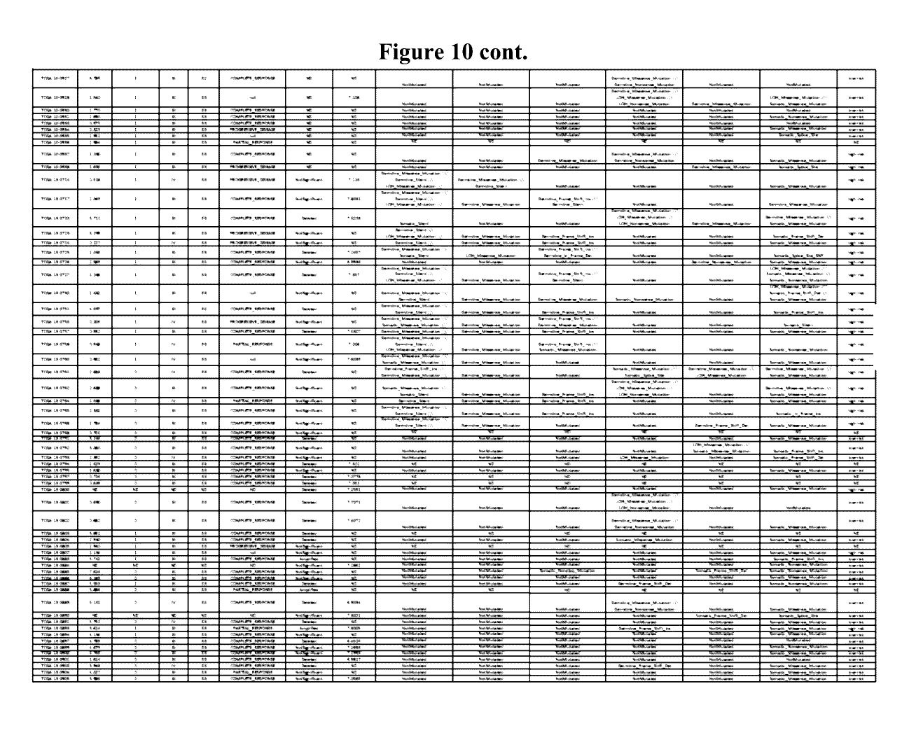

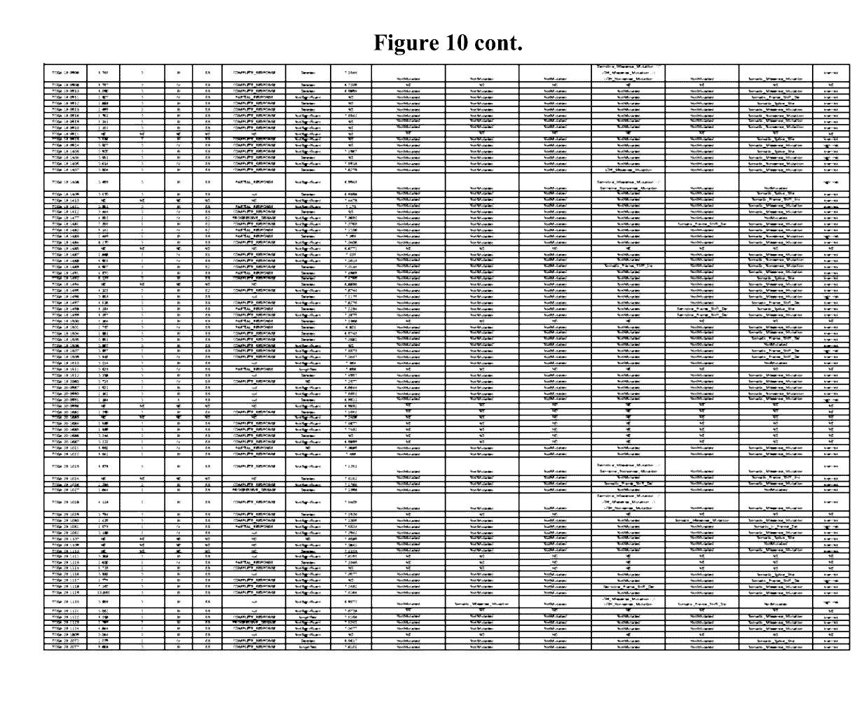

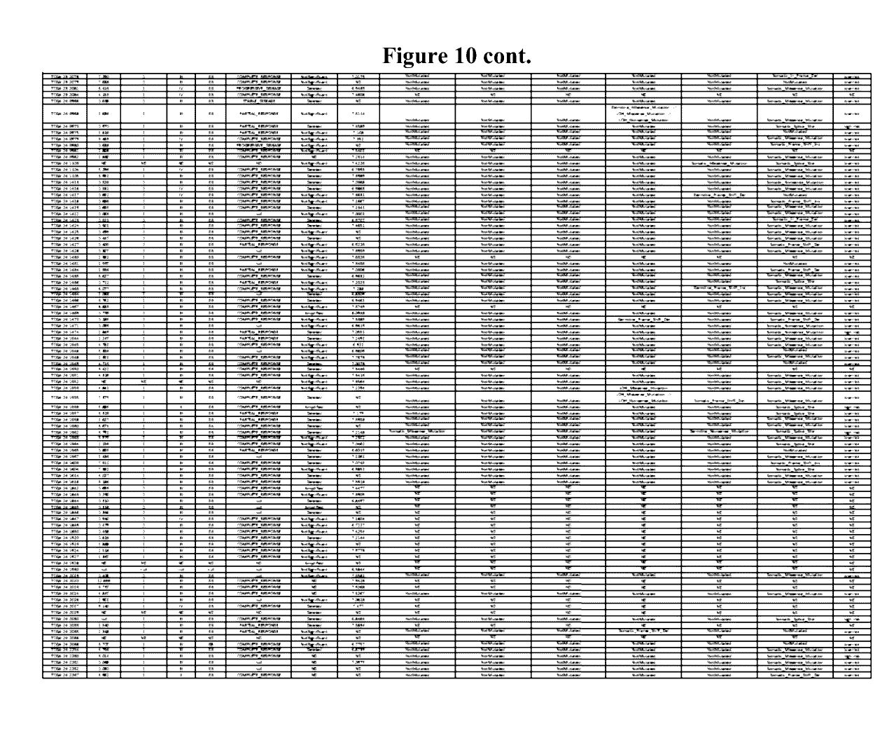

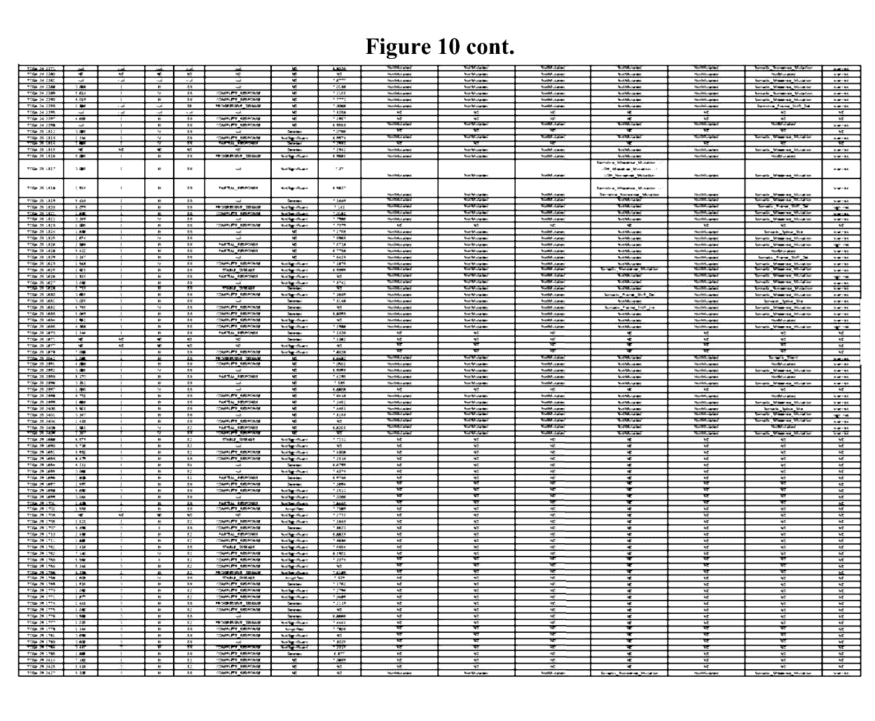

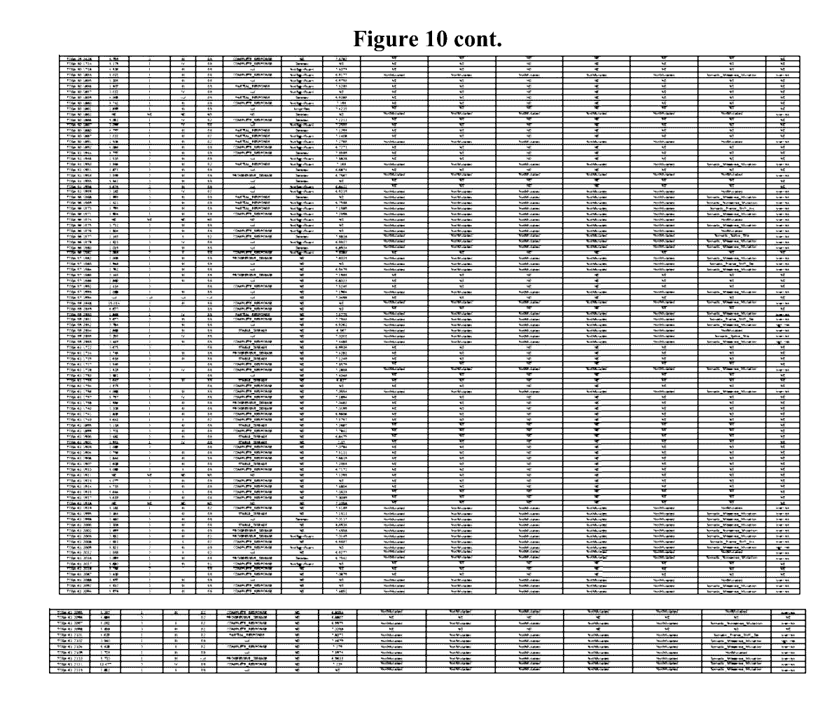

Stratification of the TCGA HG-SOC patients was performed based on the non-silent mutational status of the CHEK2 gene. In this analysis, a total of 311 patients with both mutational data and clinical information were studied (FIG. 10). Non-silent CHEK2 mutations were observed in 22 TCGA HG-SOC patients whereas mutations of CHEK2 in the remaining 289 patients (with clinical information) were not observed. Kaplan-Meier survival curve of the patient subgroup with CHEK2 mutations exhibited significantly poorer overall survival times when compared with the subgroup with no CHEK2 mutations (p-value≦0.01, FIG. 11A). Effectively, the results from retrospective study of TCGA data suggest that for patients already diagnosed with HG-SOC, non-silent mutations (germline, LOH or somatic) of the CHEK2 gene were greatly detrimental for patient overall survival times as these patients did not survive beyond 5 years after initial pathologic diagnosis.

In TCGA HG-SOC data, genes such as TP53, BRCA1 or MUC16 were mutated with higher frequency than CHEK2 but unlike CHEK2, the mutational status of these genes could not independently stratify HG-SOC patients into survival significant subgroups (FIG. 11B-D). Despite the lack of statistical significance, there is some slight indication that mutation in MUC16, a known clinical biomarker of ovarian cancer, could be associated with poor patient survival. On the other hand, patient with BRCA1 mutation appears to be associated with better patient survival which is consistent with several other published data. While TP53 is frequently mutated in HG-SOC and could be useful in disease diagnosis, our analysis revealed that in diagnosed patients, it could not be effective as a prognostic marker of patients' overall survival times (FIG. 11B).

CHEK2 Mutations are Associated with Poor Response to Therapy

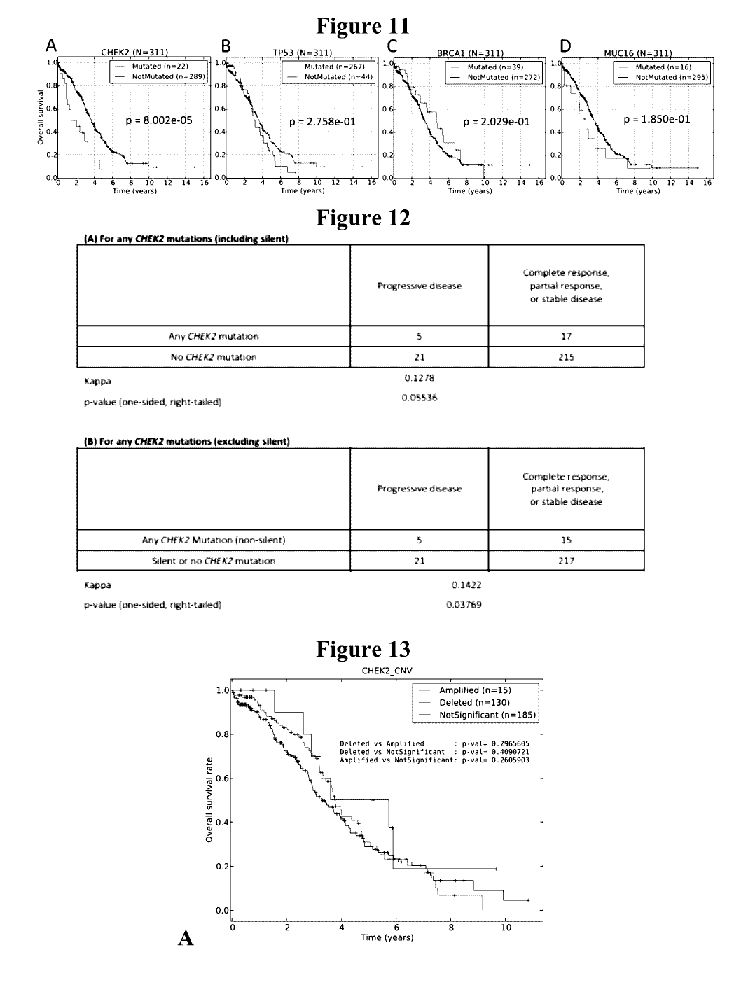

The association between CHEK2 mutations with therapy resistance were investigated and were found to be significant in HG-SOC. From the TCGA data, HG-SOC patients were categorized into two subgroups. The first subgroup consists of patients who exhibited progressive disease after primary therapy. The second subgroup consists of patients with partial response, stable disease or complete response after primary therapy. Subsequently, a two-by-two contingency table was generated where the columns represent the two subgroups of patients previously defined, and the rows correspond to the mutational status of CHEK2. Analysis via kappa correlation measure revealed that mutations in CHEK2 gene were associated with progressive disease with borderline significance (kappa=0.1278, p-value=0.05536, FIG. 12A). When silent mutations were excluded from the analysis, a slightly more significant correlation with therapy resistance was observed (kappa=0.1422, p-value=0.03769, FIG. 12B). Essentially, 25% of patients with CHEK2 mutations (5 of 20) showed disease progression whereas only 8.8% of patients without CHEK2 mutations (21 of 237) showed disease progression. Therefore, the results indicate that CHEK2 mutations are associated with poor response to therapy.

Copy Number and mRNA Expression of CHEK2 do not Appear to have Significant Influence on HG-SOC Patient Survival

To understand if other aspects of CHEK2 could be associated with patient survival, patient information for CHEK2 across available datasets from copy number, mutation, expression and clinical experiments (FIG. 10) were consolidated and subsequently assessed their prognostic significance.

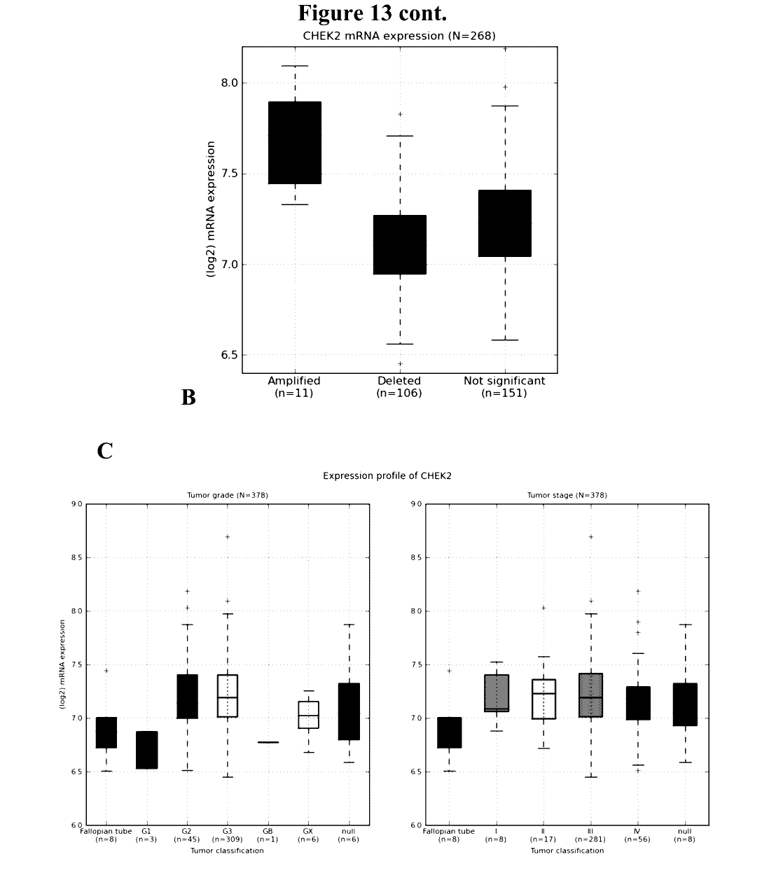

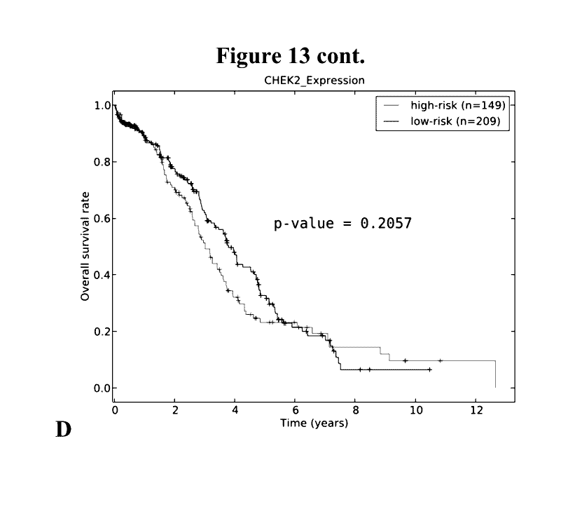

Copy number variation data was available for 356 patients. Analysis of copy number variation data for these patients revealed that CHEK2 was significantly amplified in 15 patients and deleted in 130 patients. The rest of the patients did not exhibit significant copy number variation. Subsequently, the analysis also showed that copy number of CHEK2 could not provide significant prognostic classification of HG-SOC patients (FIG. 13A). Also expectedly, samples with significant amplification of the CHEK2 region exhibited higher mRNA expression whereas those with significant deletion have lower expression (FIG. 13B).

Expression data was available for 399 samples, which comprised of 8 normal fallopian tube and 391 HG-SOC samples. Additionally, 370 of the 391 HG-SOC samples were described with tumor information such as tumor grade or tumor stage. Therefore, the expression profile of CHEK2 mRNA across the normal fallopian tube tissues and tumor tissues belonging to different grades or stages were investigated (FIG. 13C). The higher mRNA expression of CHEK2 in the tumors relative to the fallopian tube samples indicate the possible upregulation at early disease onset probably due to compensatory actions, and suggest the possibility of using CHEK2 mRNA expression as an early diagnostic biomarker for HG-SOC. On the other hand, the prognostic ability of CHEK2 expression data to classify patients already diagnosed with HG-SOC into low and high-risk subgroups is limited (FIG. 13D). A published computational algorithm was applied, which assign patients to low or high risk depending on an expression cut-off that was optimized by maximizing the separation of the two Kaplan-Meier survival curves, to CHEK2 mRNA expression belonging to the 391 HG-SOC patients. While 370 of 391 samples were annotated with clinical information, 12 were incomplete as they were without survival times and events. Therefore, survival analysis was performed on the 358 HG-SOC samples well annotated with clinical data. The results suggest that mRNA expressions of CHEK2 were not significantly associated with HG-SOC patients' prognosis (p-value=0.2057, FIG. 13D). Therefore, the results suggest that other aspects of CHEK2 such as expression or copy number variation 258 could not be used as prognostic features for HG-SOC patients.

### Observed Mutations of CHEK2 are Unlikely to Alter Phosphorylation Events or Protein Structure

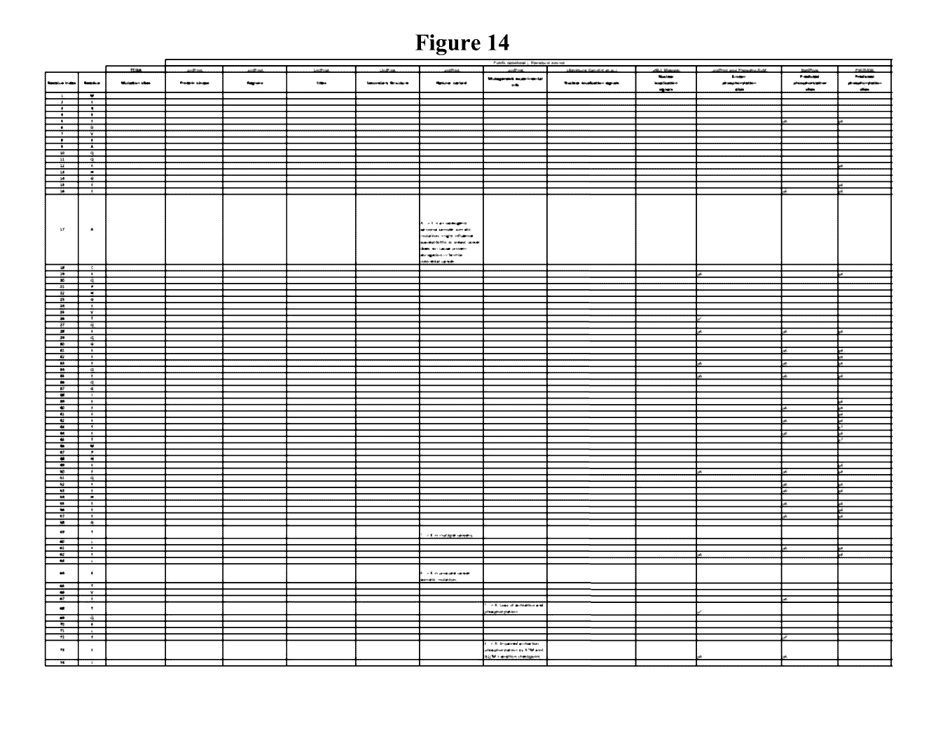

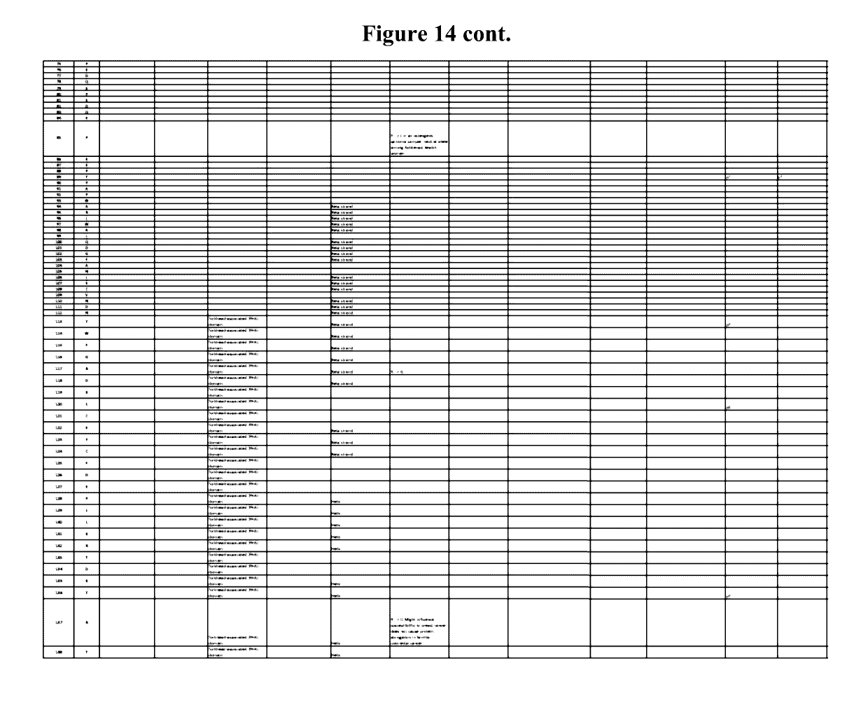

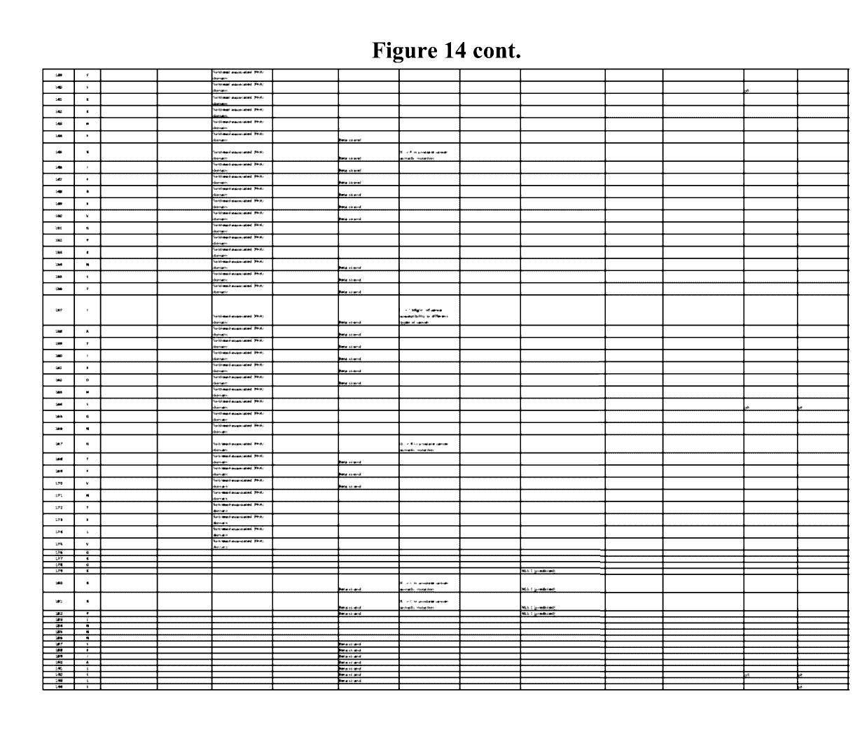

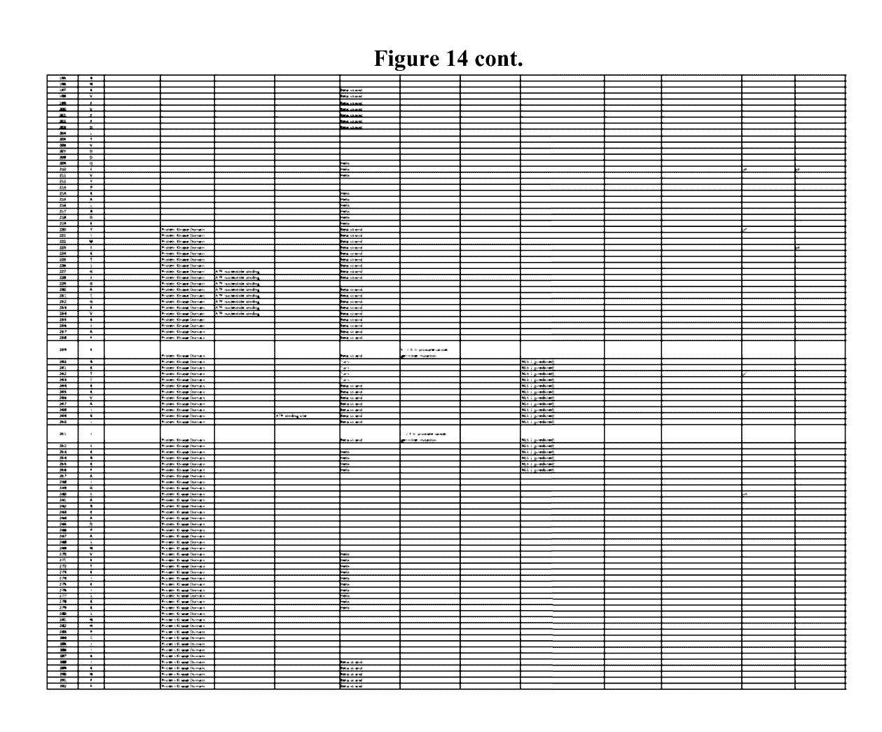

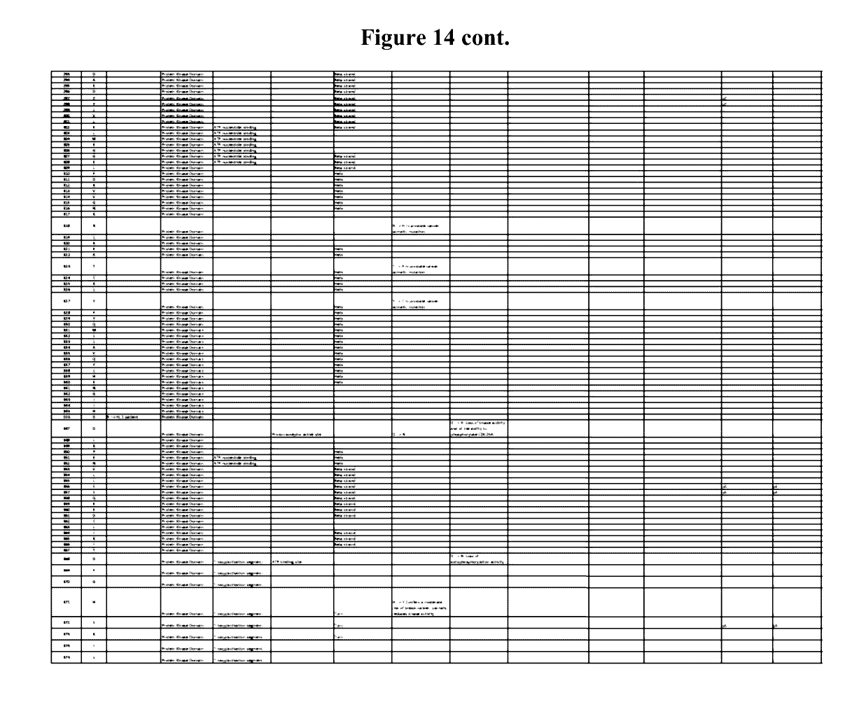

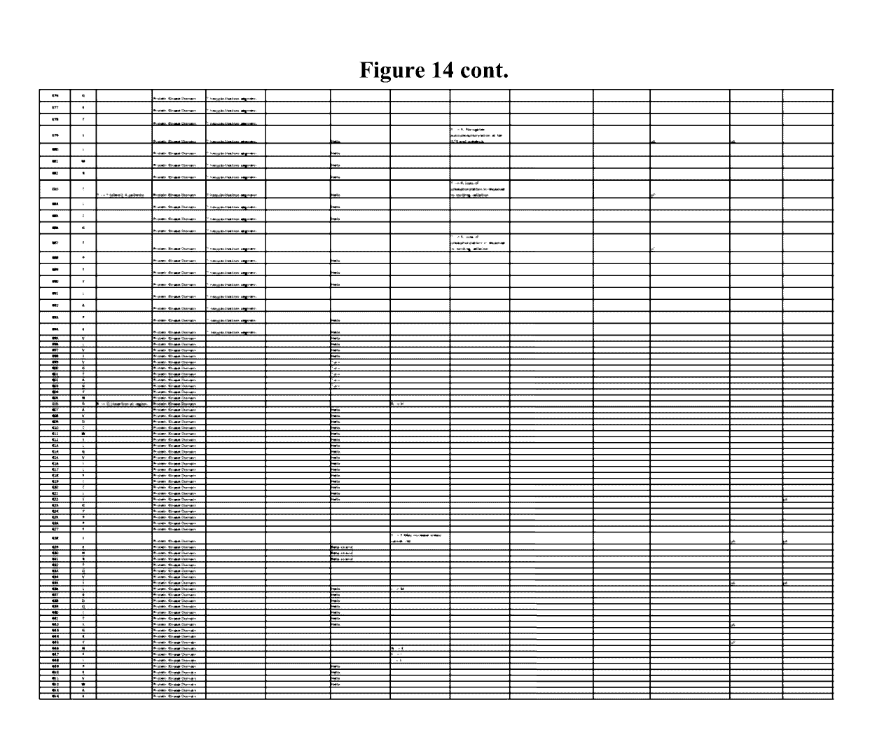

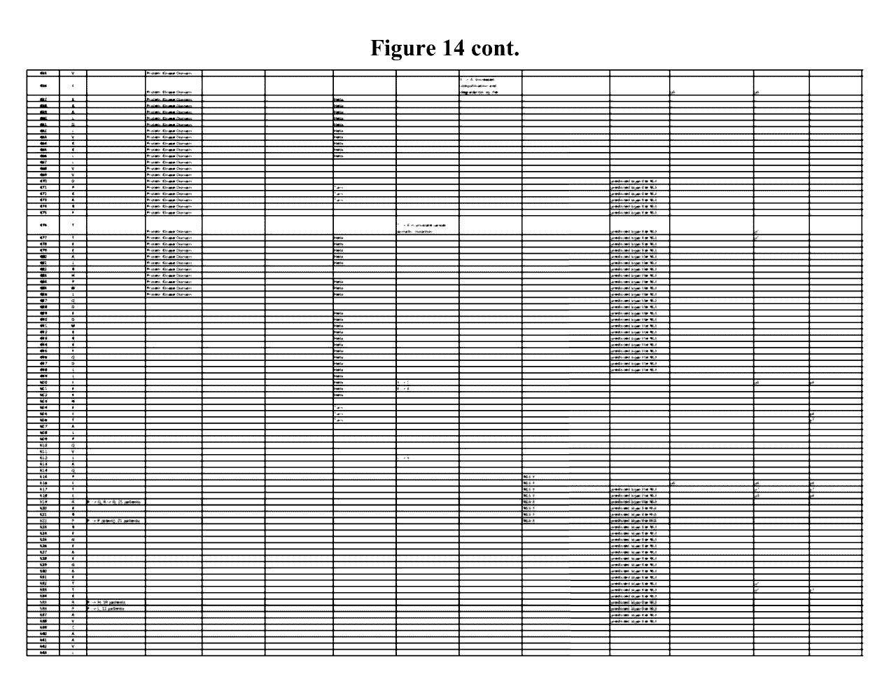

CHEK2 is a serine/threonine-protein kinase which functions in the nucleus to regulate cell cycle, DNA repair and apoptosis in response to DNA double-strand breaks. As post-translational activation of Chk2 protein via phosphorylation events is required for its physiological function, the CHEK2 mutations were checked to determine if any were localized at known or predicted phosphorylation sites. Known phosphorylation sites of CHEK2 were collected from the databases of UniProt30 and Phospho.ELM. Of all the mutations reported for CHEK2, only one mutation site was found to co-localize with a known phosphorylation site (FIG. 14). Mutation data from TCGA HG-SOC patients revealed that CHEK2 was mutated at a nucleotide coding for residue Thr-383 (hg18, chr22:27421808-27421808 at exon 11). It has been reported that auto-phosphorylation of Chk2 at Thr-383/Thr-387 within the activation loop of Chk2 kinase domain and at Ser-516 at the C-terminal region of Chk2 are essential for Chk2 activation. However, mutations at the nucleotide coding for Thr-383 were observed in only 6 patients. Furthermore, as the mutations are synonymous, the same amino acid residue threonine would be coded and therefore, it does not currently appear that mutations here would lead to aberration of Chk2 function. The results also showed that CHEK2 mutations are not co-localized with any other phosphorylation sites computationally predicted by NetPhos and PHOSIDA33, 34 274 (FIG. 14), which suggest that based on our current results, alteration of phosphorylation events may not be the key mechanisms leading to altered Chk2 behavior.

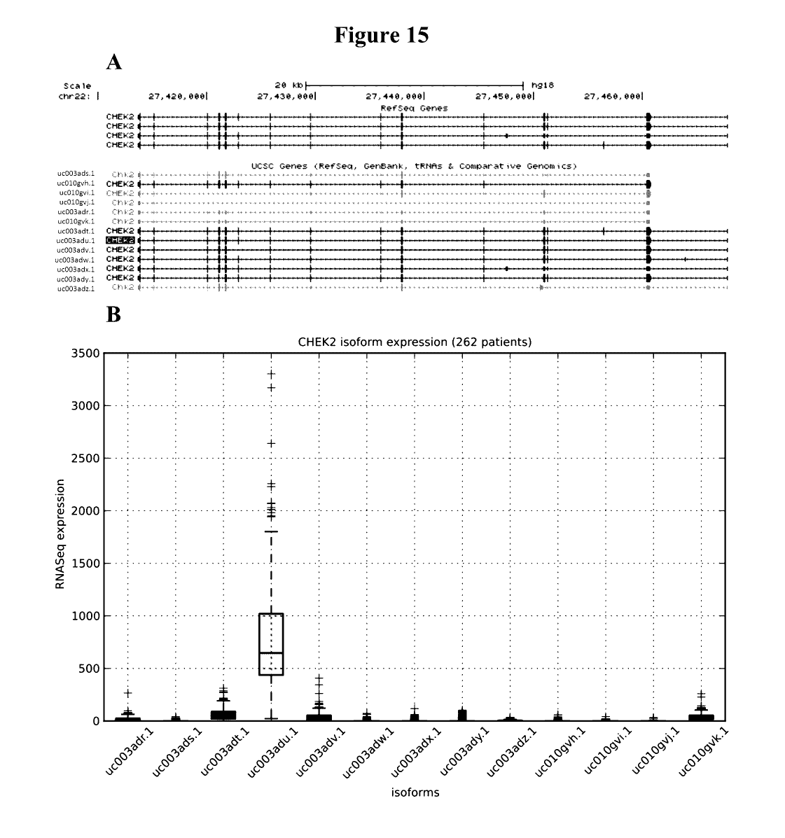

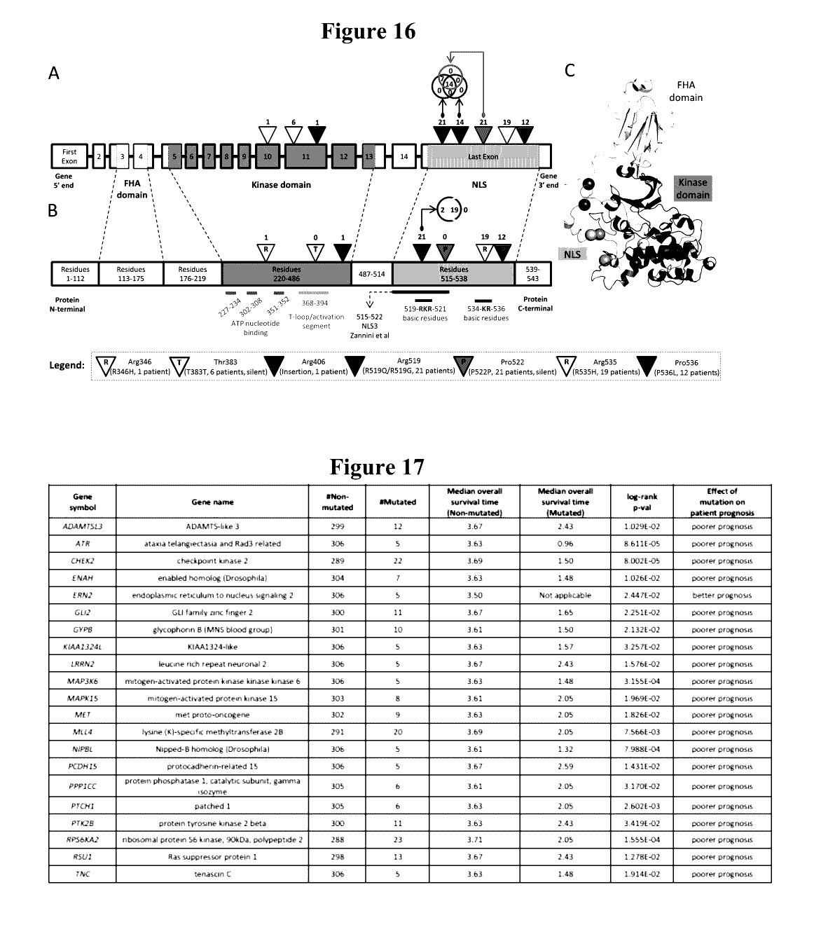

It was determined if the observed DNA mutations along CHEK2 could potentially modify the protein structure. Using data generated from RNA-sequencing experiments and downloaded from the Sage Bionetworks' Synapse database, the expression data across various CHEK2 isoforms and primary solid tumors belonging to 262 patients were first examined. An isoform uc003adu.1 was identified (representing isoform 1 or A) to be dominantly expressed when compared to other CHEK2 isoforms (FIG. 15). Known secondary structures along the amino acid residues (isoform 1, UniProt ID: 096017) were collected and compared it with the observed DNA mutations (FIG. 14). The DNA mutations at the 8 distinct sites of CHEK2 DNA could potentially alter the protein structure at 7 distinct amino acid residues (FIG. 16A-B). Only one of the amino acid residues (Thr-383) occurred at a structured site of the protein. However, the secondary helix structure is unlikely to be disrupted as the DNA mutation at this region was silent. For further visualization, a representative protein crystal structure of physiological Chk2 and superimposed the 7 mutated residues was generated to study the sites of mutations, relative to its surrounding three-dimensional conformation (FIG. 16C). From the initial crystallographic structure of Chk2 from Thr89 to Glu501 (PDB code 3i6u36, resolved at 3.0 A), Modeller was used to complete the few missing loops and to extend the C-terminal region of the kinase until Leu543. Molecular dynamics (MD) simulations were performed to obtain the relaxed state conformation of the protein structure at 50 ns (FIG. 16C). From the figure, it could be observed that the mutated residues (represented by coloured spheres) were mostly located at non-structured regions of the protein. Therefore, our results from protein modeling and MD simulations suggest that DNA mutations of CHEK2 were unlikely to disrupt the protein structure and affect its physiological function.

Observed Mutations of CHEK2 could Affect Nuclear Import of the Protein

Modifications of the NLS signals were investigate were possible among these TCGA HG-SOC patients. It has been previously reported that NLS3 is the key NLS involved in the nuclear localization of Chk2 in cells (Zannini et al. J Biol Chem. 2003 278 (43): 42346-51). The monopartite NLS3, which was computationally predicted via PSORT II, occupies a stretch of short amino acids, spanning from residues 515-522 (amino-acid sequence: PSTSRKRP, FIG. 16B) of the protein. Mutation studies performed by Zannini et al. showed that mutation of this region resulted in the cytoplasmic localization of Chk2 protein which suggested the inability of altered Chk2 in translocating to the nucleus. Interestingly, the results reveals that along this short NLS sequence, there were 3 distinct nucleotide (corresponding to 2 amino acid residues—R519 and P522) sites of mutation belonging to TCGA HG-SOC patients (FIGS. 14 and 16). The mutation observed at chromosomal coordinate chr22:27413951 was a silent mutation observed in 21 patients (P522P, labelled grey in FIG. 16A). The two non-silent mutations at contiguous nucleotide positions chr22:27413961 and chr22:27413962 were present in 14 and 21 patients respectively (R519Q/R519G, labelled dark grey in FIG. 16A). In total, 21 patients were observed to exhibit mutations at either these 2 sites and together with findings that CHEK2 mutation are detrimental to patient's survival, the results suggest a possibility that mutations in the NLS region could adversely affect the nuclear import of Chk2, reduce the protein level of effective and functional Chk2, impact Chk2-associated repair pathways, and eventually contributing to poor patient survival.

As there were two other mutation sites downstream of the NLS3, an alternative computational tool, cNLS40 was used to predict NLSs along the Chk2 protein sequence (isoform A, NP_009125—543 amino acid residues). Results revealed the possibility of a functional bipartite NLS from amino acid residues 517-538 (TSRKRPREGEAEGAETTKRPAV, FIG. 16B). This region encompasses two basic residue cluster connected by a 12 amino acid residue linker. Interestingly, of the 21 TCGA HG-SOC patients observed with mutations at the nucleotide coding for R519, concurrent mutations of nucleotide coding for R535 were observed for 90% (19 of 21) of the patients (FIG. 16B). Results from this analysis suggest that the effective NLS region could be longer than the NLS3 identified by Zannini et al. Moreover, the co-occurrences of mutations coding for residues at both key components (basic residues) of a bipartite NLS further implied the possibility of positive clonal selection in the tumor tissue samples of these 19 HG-SOC patients.

### Identification of a 21-Gene Prognostic Signature

The prognostic significance of the mutational status of 282 genes was studied with observed mutations in at least 5 patients with clinical information. The results revealed that there are 21 genes that were non-silently mutated in at least 5 patients and can independently stratify HG-SOC patients into prognostically significant subgroups (p-value≦0.05, FIG. 17). The top 3 mutated genes with prognostic significance among these 21 genes include CHEK2, RPS6KA2 and MLL4 with non-silent mutations in 22, 23 and 20 patients respectively (FIG. 17). Interestingly, the results from hierarchical clustering also revealed that these genes are clustered together (FIG. 6A). Quantitatively, kappa correlation analysis further revealed the high co-occurrences of CHEK2 mutations with RPS6KA2 or MLL4 mutations (kappa≧0.75, p-value≦5E-20, FIG. 3).

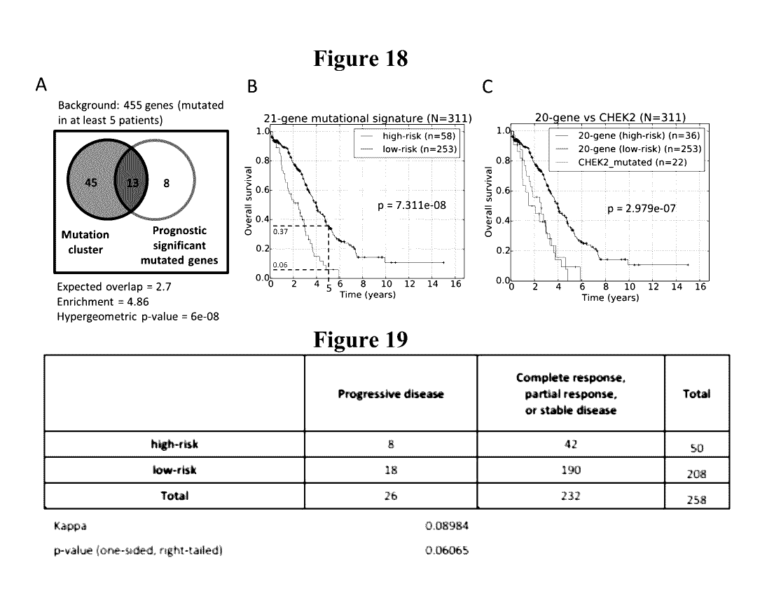

Overall, a considerable overlap of these prognostic significant genes with the genes of the CHEK2-associated mutation sub-cluster was observed (p-value=6e-08, FIG. 18A). As patients with CHEK2 mutations will generally also have mutations of genes in the mutation cluster (FIG. 6A), only the 21 prognostic genes were reviewed to create a combined mutational prognostic signature. Among these 21 genes, the mutation status of 20 genes exhibited pro-oncogenic behaviour where mutations were associated with poorer overall survival (FIG. 17). In contrast, only ERN2 exhibited tumor suppressive behaviour where mutations were associated with better overall survival. Using these genes, patients were classified into the lower risk subgroup if there were mutations in ERN2 or there were no mutations in all the 20 pro-oncogenic genes. On the other hand, patients with mutations in any of the 20 pro-oncogenic genes and without ERN2 mutations were classified as higher risk. Results from the Kaplan-Meier survival plots revealed that the 21-gene signature-defined patient subgroups were significantly stratified and associated with overall survival times (p-value=7.31e-08, FIG. 18B). Specifically, the 5-year overall survival rates of the low and high-risk subgroups are 37% and 6% respectively. To investigate if the prognostic significance of the 21-gene signature (FIG. 18B) being due to the contribution of CHEK2 mutations alone, patients with CHEK2 mutations were excluded from the signature-defined high-risk subgroup. Our results indicated that patients diagnosed with either CHEK2 mutations, or mutations in any of the remaining 20-genes signature (excluding CHEK2) exhibited rather similar overall survival patterns (FIG. 19C). The poor prognosis of patients exhibiting mutations in any of the genes in the 20-genes signature suggest that the aberrant functioning of these genes in the HG-SOC genome could inversely impact patients' post-surgery response to therapy, independent and regardless of the effects of CHEK2 mutations.

The clinical characteristics of these two subgroups of patients revealed that high-risk patients defined by the 21-gene signature are correlated with progressive disease. Specifically, patients defined as high-risk by the 21-gene mutational signature was twice as likely to exhibit progressive disease in contrast to the low-risk subgroup (high risk: 8 of 50 patients=15%; low-risk: 18 of 208 patients=8.7%, FIG. 19). However, the statistical significance is borderline (kappa=0.08984, p-value=0.06065). Nevertheless, the trend suggests that these genes could be important factors in therapy resistance.

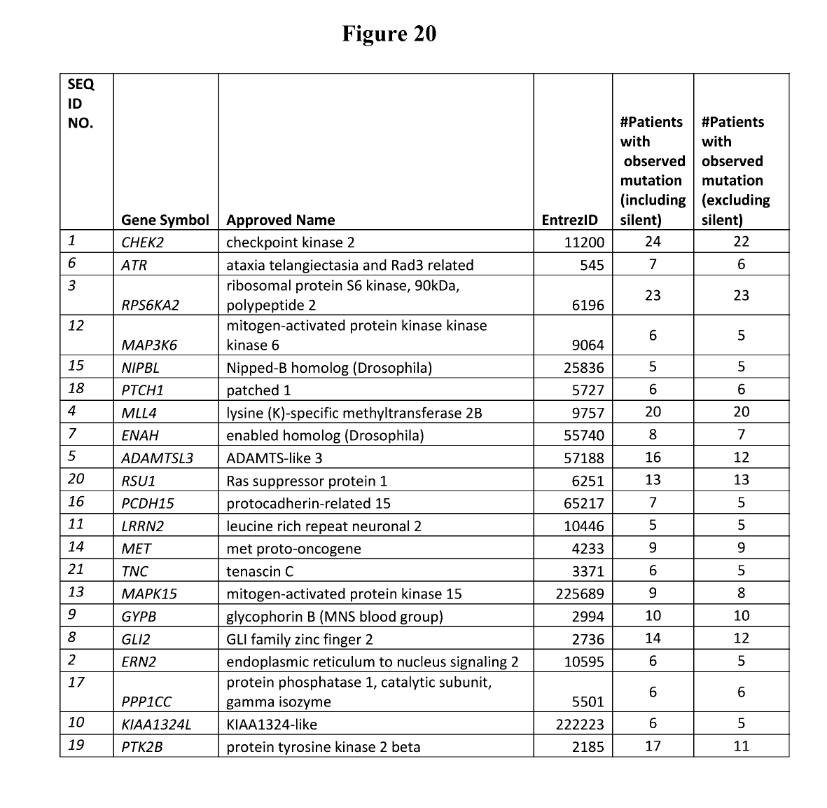

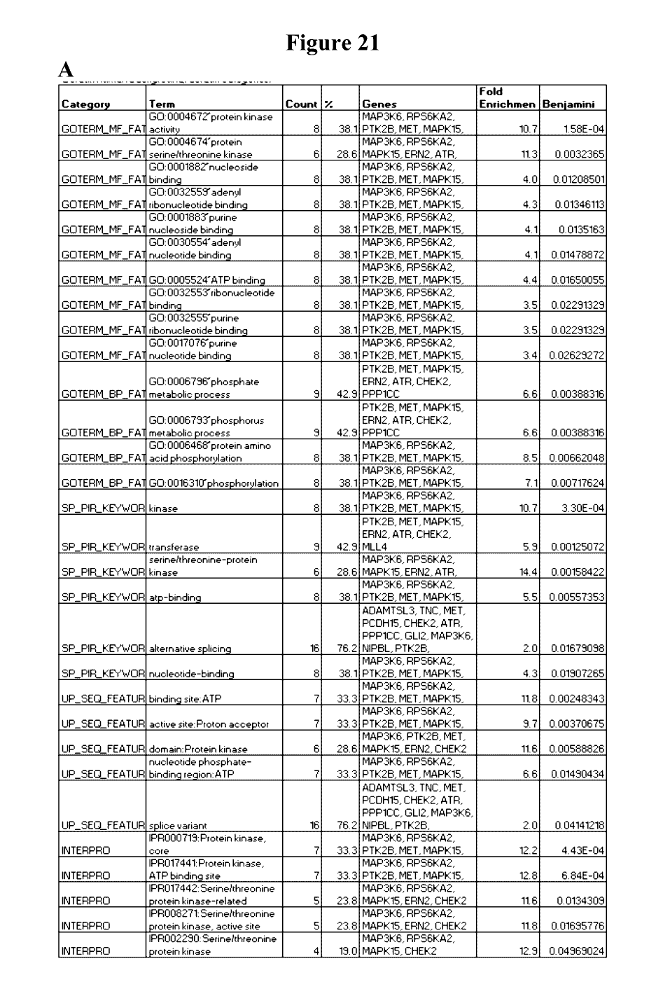

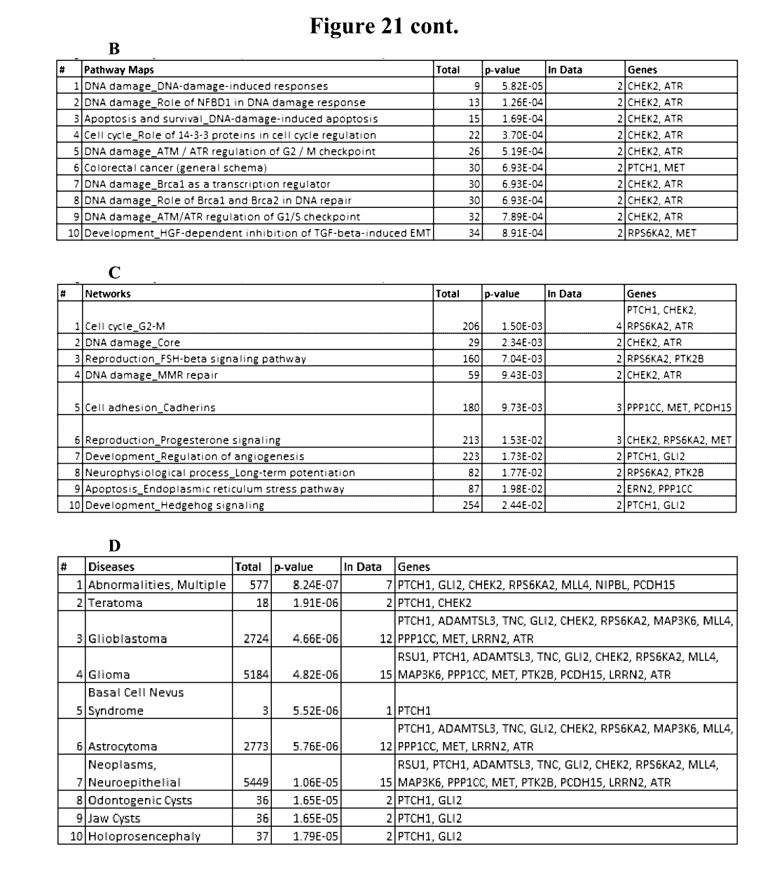

The detailed annotations of the genes in the 21-gene prognostic signature are listed in (FIG. 20). Subsequently, gene ontology analysis of the 21 genes of the signature using DAVID Bioinformatics was performed. Results indicate that these genes are strongly enriched in functions associated with kinase activity, ATP-binding and phosphorylation (FIG. 21). In parallel, analysis via MetaCore also revealed association of pathways associated with DNA damage induced responses, as well as gene networks associated with cell cycle, DNA repair, and apoptosis (FIG. 21B-C).

Identification of Two Tumor Subclasses from the Signature-Defined High-Risk Subgroup

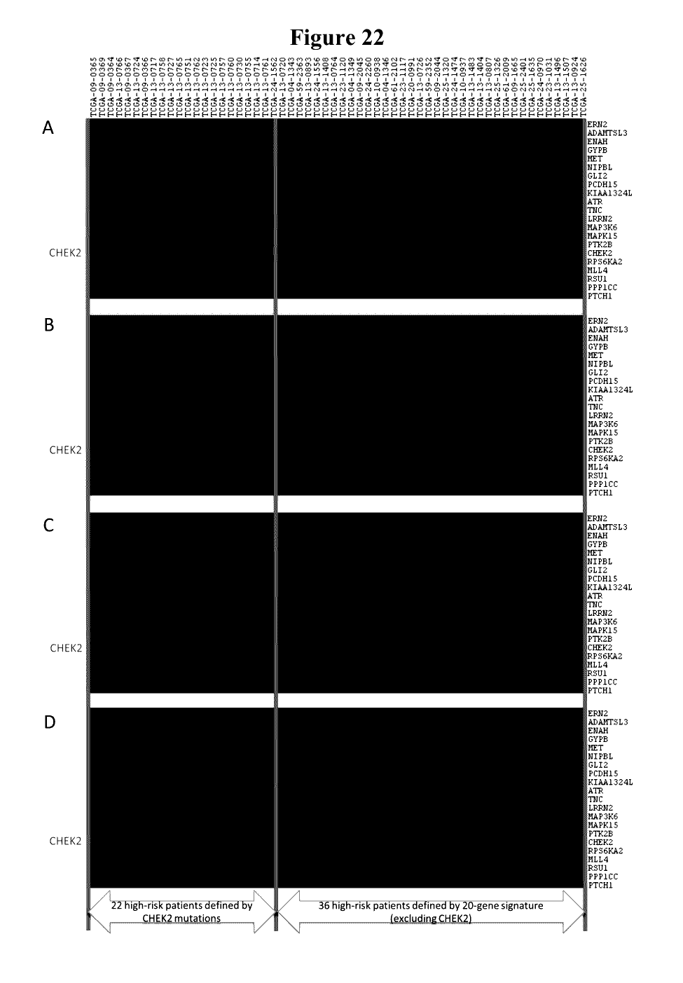

To study the possible heterogeneity of the poor prognosis patient subgroups identified via CHEK2 or the 20-gene signature, a gene-patient mutation matrix for the 21 genes and 58 patients defined as high-risk was generated (FIG. 22A). These mutations were further characterized in terms of germline, LOH or somatic mutations and our results showed that the mutations of genes in a subset of 22 patients with CHEK2 mutations appeared to be of germline or LOH origin whereas that of the other 36 high-risk patients appeared to be somatic (FIGS. 22B-D). Specifically, in the subset of 22 patients with CHEK2 mutations, 16 of the patients (73%) exhibited non-silent germline CHEK2 mutations (FIG. 23). Interestingly among these 16 patients, strong co-occurrences of germline mutations in RPS6KA2 and MLL4 genes were observed in 15 (94%) and 12 (75%) patients respectively. Re-analysis of the entire gene-patient mutation matrix (of 455 highly mutated genes and 334 patients) also revealed similar findings that genes from the CHEK2-associated mutation 410 sub-cluster were associated with germline or LOH rather than somatic mutations (results not shown).

Our results revealed that among the high-risk patients identified via our 21-gene signature, there could be two distinct tumor subclasses whose initial pathogenesis could be driven by either inherited germline mutations of CHEK2, RPS6KA2 and MLL4, or spontaneous somatic mutations of the other signature genes.

Mutations of CHEK2 in HG-SOC could Affect Nuclear Localization and Lead to Poor Clinical Outcomes

Many published mutational studies focus only on specific classes of mutations such as somatic or germline variants. The focus on germline or somatic mutations would be appropriate for specific studies when one is interested in inherited risk of developing a particular disease upon birth, or identification of driver mutations for disease development at later stages of life respectively. For prognosis purpose, whether the mutation is due to early inheritance or later stage environmental factors is of less relevance. As such, all classes of mutations were included during prognosis stratification.

Interestingly, HG-SOC patients that carry Chk2 mutations are at higher risk of mortality. But importantly, it could also prompt further studies into alternative targeted therapy for these patients. A possible explanation of why Chk2 mutations are associated with adverse patient prognosis could be due to induction of chemo-resistance, as significant correlation was found between CHEK2 and therapy response (kappa=0.1422, p-value=0.03769, FIG. 12B). None of the observed mutations in TCGA HG-SOC patients occurred in annotated secondary structures, ATP-binding site, active site, FHA domain or kinase domain (FIG. 14). Therefore, there appears to be insufficient evidence that CHEK2 mutations observed in TCGA HG-SOC patients could disrupt the protein structure, dimerization process or kinase activity and contribute to chemo-resistance.

In ovarian cancer, cisplatin is used as the main chemotherapeutic agent. Zhang et al. reported that cisplatin treatment could degrade Chk2 protein and the reduced level of Chk2 could hinder cell-cycle control, prevent cell apoptosis, and contribute to chemo-resistance of the tumors. Chk2 degradation may be one of the primary mechanism by which a large number of clinically relevant tumors develop the acquired resistance to DNA damage agent. With regards to the patients who exhibited CHEK2 mutations, the loss of function of one copy via either somatic or germline mutation, could result in reduced copies of CHEK2 in the nucleus, and subsequently upon cisplatin treatment, the effects of CHEK2 degradation could be accentuated and ultimately detrimental for patient survival. Interestingly, the reason why the observed mutations of CHEK2 might initially contribute to loss of protein functions could be attributed to the lack of protein localization in the nucleus. The lack of nuclear localization of Chk2 is likely to contribute to deviation from physiological activity and leads to undesirable effects. The analysis revealed that in 21 HG-SOC patients of the TCGA cohort, CHEK2 mutations occurred within a nuclear localization signal critical for nuclear import of the protein (FIG. 16B). Mutations of the nuclear localization signal inhibit nuclear import of the Chk2 protein, leading to reduced functional copies of Chk2 in the nucleus. It appears plausible, that mutations along the nuclear localization signal of the CHEK2 gene could lead to reduced levels of Chk2 proteins in the nucleus and upon cisplatin treatment, the protein levels would be further depleted, which could potentially lead to enhanced proliferation of the tumors in the presence of cisplatin mimicking chemo-resistance and resulting in adverse patient survival. The results showed that HG-SOC patients who exhibited CHEK2 mutations were significantly associated with poor clinical outcomes and did not survive beyond five years after initial diagnosis (FIGS. 11A and 12).

### Observed CHEK2 Mutations are Unlikely to Affect Post-Translational Modifications

CHEK2's association with poor patient prognosis was examined to see if the mutation could be due to modification of the phosphorylation sites. However, none of the observed mutations in CHEK2 occur along any currently known and annotated phosphorylation sites. Therefore, computationally identified phosphorylation motifs from the literature were collected and investigated if any of the key residues along the motifs are mutated in TCGA HG-SOC patients. The results revealed that despite their close proximity, none of the observed mutations occurred at the phosphorylation sites or the key motifs surrounding the phosphorylation sites. Furthermore, the analysis revealed that the region surrounding the CHEK2 mutations does not seem to contain strong protein secondary structure, and therefore it may currently seem unlikely that aberrations of post-translational modification of the Chk2 protein are contributory factors leading to poor survival prognosis of affected patients. However, the effect of CHEK2 mutations on protein dimerization or physical interaction with other protein partners could be investigated further.

### Possible Influences of Silent Mutations

While it is hypothesized that the mutations observed along the CHEK2 could affect nuclear translocation of the translated protein, other mechanism involving silent mutations could also be involved.

It was observed that 21 HG-SOC patients exhibited silent mutations at chr22:27413951 (P522P, FIG. 16). Traditionally, silent DNA mutations which encode for the same amino acid residues were assumed to have negligible effect on a protein function. However, recent studies have suggested that silent mutations could affect downstream protein functionality via various mechanisms. For instance, alterations to the DNA triplet codon could alter the binding sites of miRNA, leading to alteration in translational repression efficiency and downstream signal networks. It was investigated whether the mutation at chr22:27413951 (P522P, FIG. 16) could potentially alter miRNA binding sites, the results from sequence alignment indicate that the specific region was not targeted by any of the currently known human mature miRNAs (results not shown). Therefore alteration of miRNA target sites via synonymous mutation is unlikely to have any effect on mRNA stability and its subsequent translation in the mutations examined.

Single synonymous DNA mutation can affect mRNA secondary structure, folding, stability and consequently, the regulation of the translated protein as was reported for the human dopamine receptor D2 gene. It was also suggested that synonymous mutations could affect translational efficiency of the amino acid residue due to the variation and asymmetry of tRNA abundance in cells. Even in cases where synonymous mutations do not affect mRNA or protein levels, the function of the translated protein could be altered. In MDR1 gene, it was shown that a synonymous polymorphism resulting in a rare triplet codon can alter substrate specificity of the MDR1 protein, possibly due to deceleration of the translation rate at that amino acid residue which in turn affects protein folding. The strong overlap in 14 common patients exhibiting both the silent mutation and non-silent mutation at the last exon (FIG. 16A) appears to suggest a possible positive selection of these mutations and future studies could focus on elucidating the possible influence of silent mutations on eventual protein expression and functionality.

### Potential Clinical Application of the 21-Gene Mutational Signature for Prognosis and Therapeutics Design

While CHEK2 mutation appears to be the most important with respect to patient classification based on their survival patterns, a total of 21 genes were identified which could independently and significantly stratify patients into low and high-risk subgroups based on their mutational status (FIG. 17). Applying the 21 combined gene classifier to the TCGA patient cohort resulted in significant stratification of patients into two survival significant subgroups where the 5-yr overall survival rates for the low and high-risk subgroups are 37% and 6% respectively (p-value=3.8E-09, FIG. 18B). Furthermore, stratification based on the mutational status of CHEK2 alone, or of the remaining 20-gene signature permitted rejection of the hypothesis that the prognostic value of the 21-gene signature was due to the contribution of CHEK2 alone (FIG. 18C). Rather, this shows that a poor prognosis subgroup could be identified based on a 20-gene signature even in the absence of CHEK2 mutations. For prognosis purpose, while the use of the 21 gene mutational signature in patient risk prediction from the retrospective study of the TCGA patient cohort is useful, prospective studies would validate the use of the signature in a clinical setting.

Interestingly, amongst the 21 genes whose mutational status were most suitable for prognostic applications, gene functions associated with protein kinase activity, ATP-binding, phosphorylation, DNA damage response, apoptosis, or cell cycle regulation were enriched (FIG. 21). Mutations in kinases such as CHEK2 or RPS6KA2 were found to be associated with patient survival and patient stratification. Patients with characterized mutations of CHEK2 or RPS6KA2 only represent a subset of the HG-SOC patients. Most HG-SOC patients were characterized by mutations in only a few genes (FIGS. 4 and 22), which is consistent with the general consensus that patient-gene mutational profiles are heterogeneous and sparse. Nevertheless, it has been postulated that individual patients could exhibit mutations in different genes that are functionally related via gene networks corresponding to cancer hallmarks. Therefore, any particular biological process could be impacted via aberrations of any of its member genes. The understanding of the heterogeneous nature of mutations in HG-SOC patients could present an opportunity for more effective and targeted treatments in future.

### Potential Clinical Application of the 21-Gene Mutational Signature for Risk Prediction of Developing HG-SOC

Analysis of 58 high-risk HG-SOC patients identified via the 21-gene signature also revealed two distinct tumor subtypes which could arise from two different tumor etiological factors. The first tumor subclass (or patient subgroup) was clearly characterized by germline mutations or LOH of genes such as CHEK2, RPS6KA2 and MLL4 (FIG. 22). In contrast, for the other tumor subclass (or patient subgroup), germline mutations of these genes were not observed. Rather, this tumor subclass appears to be the result of spontaneous somatic mutations of these genes in the presence of TP53 mutations that typically characterized HG-SOC tumors.

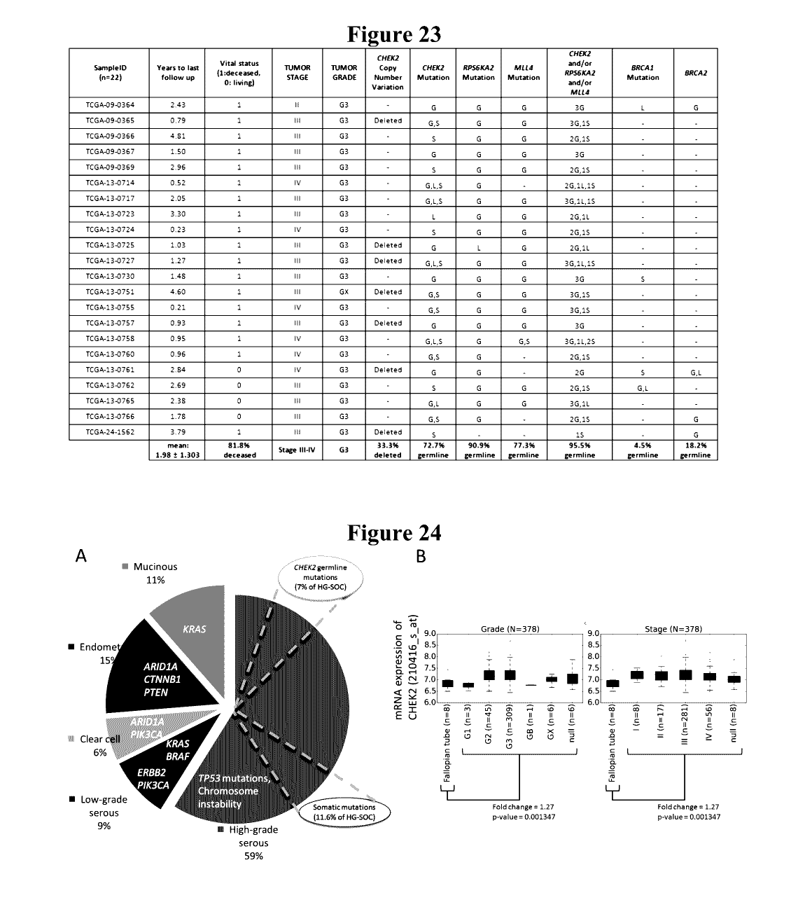

In fact, ovarian cancer is highly heterogeneous with various driver genes involved in the development of several cancer subtypes (FIG. 24A). The results suggest that around 11.6% of HG-SOC tumors could possibly be initiated due to spontaneous somatic mutations of the genes in the signature in the presence of TP53 mutations. Also, within high-grade serous ovarian cancer that is characterized by TP53 point mutations and genome instability, inherited germline CHEK2 mutations may confer susceptibility, and be involved in the initiation, development and progression of tumors in about 7.1% of HG-SOC patients (FIG. 24). The results also suggest the possibility that germline mutations of CHEK2, RPS6KA2 or MLL4 could be used as risk factors to predict a person's risk of developing HG-SOC. For CHEK2, studies have been conducted to study the effect of gene variants on ovarian susceptibility but associations were reported to be insignificant, possibly due to the rare occurrence of CHEK2 mutations, small tumor sample size, lack of appropriate HG-SOC patient samples, or low resolution of variant detection of available samples. Nevertheless, the current results from a high quality HG-SOC dataset from TCGA has uncovered a previously uncharacterized association of disease susceptibility due to CHEK2 germline variants.

### Potential Clinical Application of CHEK2 Expression in Early Diagnosis of HG-SOC

The results revealed that CHEK2 mRNA was up-regulated in tumor samples relative to normal tissues of the fallopian tube (FIG. 24B), this suggests the possibility that elevated CHEK2 mRNA expression may be used as early diagnostic marker of high-grade serous ovarian cancer. The elevated expression of CHEK2 could possibly due to response to DNA-damage or genome instability associated with TP53 mutations in HG-SOC. The induction of apoptosis in tumor cells via inhibiting Chk2 could be beneficial in preventing uncontrollable cell proliferation which consequently could lead to better patient survival. However, a recent study found that Chk2-depletion in ovarian cancer cell lines diminished cisplatin sensitivity and raised further suspicions if Chk2 could be an effective therapeutic target in cisplatin treated HG-SOC patients.

### TCGA HG-SOC Data Source and Pre-Processing

Processed mutation data belonging to 334 TCGA HG-SOC patients were downloaded from the TCGA data portal on 24th November 2010. The sequences were generated by Human Genome Sequencing Centers (HGSCs) at Baylor College of Medicine (BCM), Broad Institute Genome Center (BI) and Genome Institute at Washington University (WUSM) based on either Illumina or ABI SOLID sequencing technologies. This release included 105 Level 2 and 91 Level 3 (BCM) patients, 172 Level 2 and 158 Level 3 patients from BI, and 88 as Level 2 and Level 3 WUSM patients.

In total, 21978 mutations spanning across 334 patients and 10489 RefSeq gene symbols were reported. 4339 mutations with unknown mutation status were removed. The remaining 17639 mutations were observed in 9083 genes and encompass variants such as insertion, deletion, SNPs and silent mutations. The clinical information corresponding to each HG-SOC patients was also downloaded.

In addition, mRNA expression data of 463 primary solid ovarian cancer tissue samples were obtained (from 11 batches of 21-47 samples each). Quality assessments were performed within each batch to identify poor quality chips. 74 poor quality chips were removed from subsequent analysis. Background correction and normalization were done within each batch. Finally, batch effects were eliminated across batches using the nonparametric ComBat software.

### Copy Number Variation Analysis

Tumor-blood paired samples downloaded from TCGA portal were used.

The blood copy number variations were used for normalization and estimation of the fold change enrichment/under representation of copy number variation data for matched tumor samples. TCGA SNP array data (CNV platform 6) were processed via PARTEK 6.5 program at the parameters recommended by the company. Using PARTEK software, genomic coordinates of the copy variation segments were identified which form the statistically significant deleted or amplified genome regions. For each tumor sample, these significant regions were mapped on the human genome coordinates and normalized fold change of such signals were visualized via USCS Genome browser custom tracks. Changed copy numbers in ovarian tumours exhibit a high level of chromosomal instability. 20573 genes overlapped with changed CN segments representing about 70% of RefSeq protein-coding genes overlapped with significant alteration copy number regions.

### Processed RNA-Sequencing Expression Data

Processed RNA-sequencing expression data of genes and the gene isoforms were downloaded from the Sage Bionetworks' Synapse database. This dataset contains RNA-seq expression data for 73598 gene isoforms and 266 samples corresponding to 263 patients. Of the 266 samples, 262 samples (from 262 patients) were collected from primary solid tumor whereas the rest were collected from recurrent solid tumor.

### Secondary Data Source

Protein annotation data comprising of important functional sites, secondary structure, natural variants, mutagenesis experimental data, and phosphorylation sites was obtained from UniProt. Additionally, known phosphorylation sites were downloaded from validated database Phopho.ELM. Phosphorylation sites were further predicted using online tools NetPhos and PHOSIDA which were based on machine learning techniques such as artificial neural network or support vector machine. Nuclear localization signals were predicted via online computational tools PSORT II and cNLS Mapper.

### Mutation Matrix Across Patients and Genes

The mutation spectrum across the patients and genes are represented in a two-dimensional matrix, M comprising of 9083 rows and 334 columns which represent gene symbols and patient sample IDs respectively. Each entry in the matrix, Mᵢⱼ represents the number of unique mutation sites in the i^{th} gene of the j^{th} patient sample.

### Analysis of the Frequency Distribution of the Number of Mutated Tumor Samples for a Susceptible Gene

The Kolmogorov-Waring (K-W) probability function is used to fit the distribution of the number of mutated tumor tissue samples. The function is described as:

$$P ( X - m ) := {p}_{m} = {p}_{o} \frac{B ( b + 1 , m )}{B ( a , m )} {θ}^{m} \qquad ( Eq 1 )$$

where m=0, 1, 2, . . . and b, a, θ are the parameters of our model. B(x) is the Beta function as previously described. In the case where b>a>0, the probability of non-observed events is estimated by the formula

$$Po = ( 1 - \frac{a}{b} ) , \qquad Eq 1$$

can be presented in the form of the following recursive formula for easy computational estimate of the model parameters:

$${P}_{m + 1} = θ \frac{( a + m )}{b + m + 1} {P}_{m} , \qquad ( Eq 2 )$$

In order to apply the probability function (Eq1) or (Eq2) to the observed data, it was assumed that the random variable X is restricted to sample size and the rarest events are non-observed. Thus, random variable X is doubly truncated, i.e., the range 1, 2, . . . , J(J<∞). Using (Eq1), the probability distribution function of the resulting truncated distribution function is written as the following:

$${P}_{m}^{T} = {P}_{m} / ( {\sum }_{s = 1}^{s = J} {P}_{s} ) = \frac{Pm}{1 - Po - PJ + 1 / Po , J + 1} \qquad ( Eq 3 )$$

This probability distribution function corresponds to a typical situation in analysis of mutagenesis data in a limited cohort where the occurrence values 0 and J+1, J+2, . . . are not detected. Details of the curve-fitting computational algorithm have been previously published.

### Hierarchical Clustering

A numerical matrix that represents the mutation pattern across patients and genes are generated. Rows and columns correspond to genes and patients respectively. Each numerical value in the matrix represents the number of distinct locations with reported mutations, for that patient and gene. Hierarchical clustering analysis was performed using Kendall-tau as the similarity metric and complete linkage as the clustering method. The mathematical procedure was implemented in Gene Cluster 3.0 and visualized via Java TreeView. The intensity of the plot corresponds with the number of distinct mutated locations for that patient and gene.

### Gene Enrichment and Network Analysis

Gene functional enrichment analysis was performed via DAVID Bioinformatics and MetaCore from GeneGo Inc. The default human genome genes were used as the background set. Default parameters were used. The gene network was generated via MetaCore via direct interacting network algorithm. The legend of the network can be assessed from http://ftp.genego.com/files/MC_legend.pdf.

### Survival Analysis

Survival analyses of patient subgroups were performed with reference to their overall survival times (years to last follow up) and survival event (vital status at last follow up). Comparative survival times and events of patient subgroups were visualized using Kaplan-Meier survival curves which represents the probability of patient survival at a given time after initial diagnosis. The statistical significance of patient subgroup stratification across the full survival time range was evaluated using the log-rank test, which is based on the chi-sq distribution. The procedures were implemented using open source R programming language and packages.

### Measure of Agreement Test

The correlations between ordered patient subgroups with clinical parameters such as therapy response were calculated using weighted kappa correlation measure. The statistical significance was estimated using Mantel-Haenszel (MH) test. The calculations were implemented using StatXact-9 (computed weight: quadratic difference, scores: equally spaced). All p-values are one-sided (right-tailed) which indicates the probability that a random kappa correlation measure is greater than actually observed.

### Protein Structure Modeling

The initial structure was taken from the crystal structure of the serine/threonine protein kinase chk2. (PDB code 3i6u, resolved at 3.0 A). The crystallographic unit contains a dimeric protein (chains A and B). The crystal construct comprises from residue Thr89 to Glu501. The program Modeller has been used to complete few missing loops and to extend the C-terminal region of the kinase until Leu543, in order to include the nuclear localization signal motif. PDB2PQR was used for protonation of residues. MD simulations were set up using the antechamber and LEaP modules in the AMBER 12 package. The system was solvated in a truncated octahedron TIP3P water box and neutralised with sodium ions. Minimization and MD simulations using the amber ff99SB allatom forcefield, were carried out with the Sander module of the Amber12 package using the GPU-accelerated version of the program. A multistep scheme was followed, as previously described. The conformation at 50 ns was extracted and assumed that along the trajectory the kinase and especially the C-termini tail have adopted a relaxed state. PyMOL has been used to visualise and generate the figures.

1. A method for determining the prognosis of a patient afflicted by high-grade serous ovarian cancer (HG-SOC), comprising determining the presence or absence of a mutation in the CHEK-2 gene and optionally a gene selected from CHEK2, ERN2, ADAMTSL3, ATR, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC in a sample obtained from said patient, wherein the presence of a mutation in the ERN2 gene is indicative for a favorable prognosis of the patient and the presence of a mutation in any one of the CHEK2, ADAMTSL3, ATR, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC genes is indicative for an unfavorable prognosis of the patient and wherein determining the presence or absence of a mutation in the CHEK2 marker comprises detecting the presence or absence of a mutation in a nucleotide sequence as set forth in any one of SEQ ID NOS. 1 and 67-69.

2. The method of claim 1, further comprising the step of confirming the prognosis by microscopic analysis of an ovarian tissue biopsy or by ultrasound.

3. (canceled)

4. The method of claim 1, wherein the mutation in the CHEK2 marker is located in exon 10, 11 or 15 of the CHEK2 marker.

5. The method of claim 1, wherein the mutation in the CHEK2 marker is located in the terminal exon expressing a nuclear localization signal sequence.

6. The method of claim 4, wherein the mutation is located at a codon sequence expressing amino acid R346, T383, R406, R519, P522, R535 and/or P536.

7. The method of claim 1, further comprising determining a mutation in any one of the genes selected from the group consisting of ABCA3, ADAM15, ALK, ANKHD1-EIF4EBP3, ANKMY2, ANXA7, ASPM, CDC27, CHD6, CHL1, DPYSL4, EP400, ERBB2IP, FN1, FOXO3, GCLC, GLI3, GZMB, HLA-G, HNF1A, INPP5D, INSR, ITGB2, KIF3B, KIF4B, KTN1, MAD1L1, MKL1, MYO5C, NUMA1, PDGFRA, PHLPP, PIK3C2B, PKP4, PLAGL2, PPARA, PRKCI, RAB3D, ROR2, SPTB, TBK1, TNK2, TP53, VAV1 and ZC3H11A.

8. The method of claim 1, wherein determining a mutation in any one of the marker genes comprises detecting the presence or absence of a mutation in a nucleotide sequence selected from the group consisting of nucleic acid sequences set forth in SEQ ID NOS 3-5, 7-9, 11-14, 19, 20, 22-73, 77-79 and 99-175.

9. The method of claim 1, further comprising determining a mutation in any one of the marker genes comprises detecting the presence or absence of a mutation in a nucleotide sequence selected from the group consisting of the nucleic acid sequences set forth in SEQ ID NOS 2-21, and 70-102.

10. The method of claim 1, comprising determining the presence or absence of a mutation in a panel of gene markers comprising CHEK2, ERN2, ADAMTSL3, ATR, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC.

11. The method of claim 10, wherein the panel of markers comprises detecting the presence or absence of a mutation in a nucleotide sequences set forth in SEQ ID NOS 1-21, and 67-102.

12. The method of claim 1, wherein the ERN2 marker comprises a nucleic acid sequence set forth in SEQ ID NO. 2.

13. The method of claim 10, wherein the presence of a mutation in the panel of markers comprising CHEK2, ADAMTSL3, ATR, ENAH, GLI2, GYPB, KIAA1324L, LRRN2, MAP3K6, MAPK15, MET, MLL4, NIPBL, PCDH15, PPP1CC, PTCH1, PTK2B, RPS6KA2, RSU1 and TNC is indicative of an unfavorable therapeutic outcome of the patient.

14. The method of claim 1, wherein a first tumor subtype is determined by a germ line mutation in CHEK2, RPS6KA2 and/or MLL4 marker.

15. The method of claim 1, wherein a first tumor subtype is determined by a germ line mutation in marker sequences set forth in SEQ ID NOS 1, 3, 4 67-69, and/or 70.

16. The method of claim 1, comprising determining the presence or absence of a mutation in a panel of gene markers comprising CHEK2, ABCA3, ADAM15, ADAMTSL3, ALK, ANKHD1-EIF4EBP3, ANKMY2, ANXA7, ASPM, CDC27, CHD6, CHL1, DPYSL4, ENAH, EP400, ERBB2IP, FN1, FOXO3, GCLC, GLI2, GLI3, GYPB, GZMB, HLA-G, HNF1A, INPP5D, INSR, ITGB2, KIF3B, KIF4B, KTN1, LRRN2, MAD1L1, MAP3K6, MAPK15, MET, MKL1, MLL4, MYO5C, NUMA1, PDGFRA, PHLPP, PIK3C2B, PKP4, PLAGL2, PPARA, PRKCI, PTK2B, RAB3D, ROR2, RPS6KA2, RSU1, SPTB, TBK1, TNK2, TP53, VAV1 and ZC3H11A.

17. The method of claim 16, wherein the panel of gene markers comprises detecting the presence or absence of a mutation in a nucleotide sequences set forth in SEQ ID NOS 3-5, 7-9, 11-14, 19, 20, 22-66, 70-73, 77-79 and 99-175.

18-31. (canceled)

32. The method according to claim 1, wherein the presence or absence of said mutation can predict patients' resistance to therapy.

33. The method according to claim 1, comprising the characterization of two distinct HG-SOC genetic subclasses, wherein one of the tumor subclasses is defined via determining the presence or absence of a germline mutation in a panel of gene markers comprising CHEK2, MLL4 and/or RPS6KA2, and wherein the second subclass is defined via determining the presence or absence of a somatic mutation in a panel of gene markers comprising ADAMTSL3, ATR, ENAH, ERN2, GLI2, GYPB, KIAA1324L, LRRN2, MAP3K6, MAPK15, MET, NIPBL, PCDH15, PPP1CC, PTCH1, PTK2B, RSU1 and/or TNC.

34. The method according to claim 13, wherein the presence or absence of a germline mutation in a panel of gene markers comprising CHEK2, MLL4 and RPS6KA2 can predict healthy individuals' inherited susceptibility to initiation, development and progression of tumors leading to HG-SOC.

35. The method according to claim 1, comprising determining the presence or absence of a mutation in a marker gene via any sequencing methods.

36. A method for determining the risk of a healthy woman to develop HG-SOC, comprising determining the presence or absence of a mutation in the CHEK-2 gene in a sample obtained from said woman, wherein determining the presence or absence of a mutation in the CHEK-2 marker comprises detecting the presence or absence of a mutation in a nucleotide sequence as set forth in any one of SEQ ID NOS. 1 and 67-69.

37. The method according to claim 36, wherein the sample is a body fluid, cell or tissue sample.
